SEQUENCE LISTING (137 sequences)

SEQ ID NO: 1 - protein, 149 aa, Homo sapiens
MKVASGSTATAAAGPSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEVRSRSDH

SEQ ID NO: 2 - protein, 134 aa, Homo sapiens
MKAFSPVRSVRKNSLSDHSLGISRSKTPVDDPMSLLYNMNDCYSKLKELVPSIPQNKKVSKMEILQHVIDYILDLQIALDSHPTIVSLHHQRPGQNQASRTPLTTLNTDISILSLQASEFPSELMSNDSKALCG

SEQ ID NO: 3 - protein, 119 aa, Homo sapiens
MKALSPVRGCYEAVCCLSERSLAIARGRGKGPAAEEPLSLLDDMNHCYSRLRELVPGVPRGTQLSQVEILQRVIDYILDLQVVLAEPAPGPPDGPHLPIQTAELTPELVISNDKRSFCH

SEQ ID NO: 4 - protein, 161 aa, Homo sapiens
MKAVSPVRPSGRKAPSGCGGGELALRCLAEHGHSLGGSAAAAAAAAAARCKAAEAAADEPALCLQCDMNDCYSRLRRLVPTIPPNKKVSKVEILQHVIDYILDLQLALETHPALLRQPPPPAPPHHPAGTCPAAPPRTPLTALNTDPAGAVNKQGDSILCR

SEQ ID NO: 5 - protein, 407 aa, Homo sapiens
MESRKDMVVFLDGGQLGTLVGKRVSNLSEAVGSPLPEPPEKMVPRGCLSPRAVPPATRERGGGGPEEEPVDGLAGSAAGPGAEPQVAGAAMLGPGPPAPSVDSLSGQGQPSSSDTESDFYEEIEVSCTPDCATGNAEYQHSKGSGSEALVGSPNGGSETPKSNGGSGGGGSQGTLACSASDQMRRYRTAFTREQIARLEKEFYRENYVSRPRRCELAAALNLPETTIKVWFQNRRMKDKRQRLAMTWPHPADPAFYTYMMSHAAAAGGLPYPFPSHLPLPYYSPVGLGAASAASAAASPFSGSLRPLDTFRVLSQPYPRPELLCAFRHPPLYPGPAHGLGASAGGPCSCLACHSGPANGLAPRAAAASDFTCASTSRSDSFLTFAPSVLSKASSVALDQREEVPLTR

SEQ ID NO: 6 - protein, 271 aa, Homo sapiens
MLLAPPSTPSRGRTPSAVERLEADKAKYVKTHQVIARRQEPALRGSPGPLTPHPCNELGPPASPRTPRPVRRGSGRRLPRPDSLIFYRQKRDCKASVNKENAKGQGLVRRLFLGAPRDAAPSSPASTERPAASGGWAAPQDAPEAAGKRALCPTCSLPLSEKERFFNYCGLERALVEVLGAERFSPQSWGADASPQAGTSPPPGSGDASDWTSSDRGVDSPGGAGGGGGSEAAGSARDRRPPVSVVERNARVIQWLYGCQRARGPPRESEV

SEQ ID NO: 7 - protein, 268 aa, Homo sapiens
MAQPLCPPLSESWMLSAAWGPTRRPPPSDKDCGRSLVSSPDSWGSTPADSPVASPARPGTLRDPRAPSVGRRGARSSRLGSGQRQSASEREKLRMRTLARALHELRRFLPPSVAPAGQSLTKIETLRLAIRYIGHLSAVLGLSEESLQRRCRQRGDAGSPRGCPLCPDDCPAQMQTRTQAEGQGQGRGLGLVSAVRAGASWGSPPACPGARAAPEPRDPPALFAEAACPEGQAMEPSPPSPLLPGDVLALLETWMPLSPLEWLPEEPK

SEQ ID NO: 8 - protein, 457 aa, Homo sapiens
MLGAVKMEGHEPSDWSSYYAEPEGYSSVSNMNAGLGMNGMNTYMSMSAAAMGSGSGNMSAGSMNMSSYVGAGMSPSLAGMSPGAGAMAGMGGSAGAAGVAGMGPHLSPSLSPLGGQAAGAMGGLAPYANMNSMSPMYGQAGLSRARDPKTYRRSYTHAKPPYSYISLITMAIQQSPNKMLTLSEIYQWIMDLFPFYRQNQQRWQNSIRHSLSFNDCFLKVPRSPDKPGKGSFWTLHPDSGNMFENGCYLRRQKRFKCEKQLALKEAAGAAGSGKKAAAGAQASQAQLGEAAGPASETPAGTESPHSSASPCQEHKRGGLGELKGTPAAALSPPEPAPSPGQQQQAAAHLLGPPHHPGLPPEAHLKPEHHYAFNHPFSINNLMSSEQQHHHSHHHHQPHKMDLKAYEQVMHYPGYGSPMPGSLAMGPVTNKTGLDASPLAADTSYYQGVYSRPIMNSS

SEQ ID NO: 9 - protein, 654 aa, Homo sapiens
MNQPQRMAPVGTDKELSDLLDFSMMFPLPVTNGKGRPASLAGAQFGGSGLEDRPSSGSWGSGDQSSSSFDPSRTFSEGTHFTESHSSLSSSTFLGPGLGGKSGERGAYASFGRDAGVGGLTQAGFLSGELALNSPGPLSPSGMKGTSQYYPSYSGSSRRRAADGSLDTQPKKVRKVPPGLPSSVYPPSSGEDYGRDATAYPSAKTPSSTYPAPFYVADGSLHPSAELWSPPGQAGFGPMLGGGSSPLPLPPGSGPVGSSGSSSTFGGLHQHERMGYQLHGAEVNGGLPSASSFSSAPGATYGGVSSHTPPVSGADSLLGSRGTTAGSSGDALGKALASIYSPDHSSNNFSSSPSTPVGSPQGLAGTSQWPRAGAPGALSPSYDGGLHGLQSKIEDHLDEAIHVLRSHAVGTAGDMHTLLPGHGALASGFTGPMSLGGRHAGLVGGSHPEDGLAGSTSLMHNHAALPSQPGTLPDLSRPPDSYSGLGRAGATAAASEIKREEKEDEENTSAADHSEEEKKELKAPRARTSPDEDEDDLLPPEQKAEREKERRVANNARERLRVRDINEAFKELGRMCQLHLNSEKPQTKLLILHQAVSVILNLEQQVRERNLNPKAACLKRREEEKVSGVVGDPQMVLSAPHPGLSEAHNPAGHM

SEQ ID NO: 10 - protein, 148 aa, Mus musculus
MKVASGSAAAAAGPSCSLKAGRTAGEVVLGLSEQSVAISRCAGTRLPALLDEQQVNVLLYDMNGCYSRLKELVPTLPQNRKVSKVEILQHVIDYIRDLQLELNSESEVGTTGGRGLPVRAPLSTLNGEISALAAEAACVPADDRILCR

SEQ ID NO: 11 - DNA, 1239 bp, Homo sapiens
  source 1..1239: mol_type = genomic DNA
actctcattccacgttcttaactgttccattttccgtatctgcttcgggcttccacctcatttttttcgctttgcccattctgtttcagccagtcgccaagaatcatgaaagtcgccagtggcagcaccgccaccgccgccgcgggccccagctgcgcgctgaaggccggcaagacagcgagcggtgcgggcgaggtggtgcgctgtctgtctgagcagagcgtggccatctcgcgctgcgccgggggcgccggggcgcgcctgcctgccctgctggacgagcagcaggtaaacgtgctgctctacgacatgaacggctgttactcacgcctcaaggagctggtgcccaccctgccccagaaccgcaaggtgagcaaggtggagattctccagcacgtcatcgactacatcagggaccttcagttggagctgaactcggaatccgaagttggaacccccgggggccgagggctgccggtccgggctccgctcagcaccctcaacggcgagatcagcgccctgacggccgaggtgagatccagatccgaccactagatcatccttataccgacggggaaacggaggccagagagggcgtgggcgcttgcaccacttccgtcccatccttgcgggtacctggctatgcgggggtgcctaaggagcctggaaaaagcgctcccccgtcgtgcttcctggggaagggggcgttcgctgcgctcggagcggcgtcccttccaacccgccggtctcatttcttctcgttttcacaggcggcatgcgttcctgcggacgatcgcatcttgtgtcgctgaagcgcctcccccagggaccggcggaccccagccatccagggggcaagaggaattacgtgctctgtgggtctcccccaacgcgcctcgccggatctgagggagaacaagaccgatcggcggccactgcgcccttaactgcatccagcctggggctgaggctgaggcactggcgaggagagggcgctcctctctgcacacctactagtcaccagagactttagggggtgggattccactcgtgtgtttctattttttgaaaagcagacattttaaaaaatggtcacgtttggtgcttctcagatttctgaggaaattgctttgtattgtatattacaatgatcaccgactgaaaatattgttttacaatagttctgtggggctgtttttttgttattaaacaaataatttagatggtggtaaaaaaaaa

SEQ ID NO: 12 - DNA, 1402 bp, Homo sapiens
  source 1..1402: mol_type = genomic DNA
ggggacgaagggaagctccagcgtgtggccccggcgagtgcggataaaagccgccccgccgggctcgggcttcattctgagccgagcccggtgccaagcgcagctagctcagcaggcggcagcggcggcctgagcttcagggcagccagctccctcccggtctcgccttccctcgcggtcagcatgaaagccttcagtcccgtgaggtccgttaggaaaaacagcctgtcggaccacagcctgggcatctcccggagcaaaacccctgtggacgacccgatgagcctgctatacaacatgaacgactgctactccaagctcaaggagctggtgcccagcatcccccagaacaagaaggtgagcaagatggaaatcctgcagcacgtcatcgactacatcttggacctgcagatcgccctggactcgcatcccactattgtcagcctgcatcaccagagacccgggcagaaccaggcgtccaggacgccgctgaccaccctcaacacggatatcagcatcctgtccttgcaggcttctgaattcccttctgagttaatgtcaaatgacagcaaagcactgtgtggctgaataagcggtgttcatgatttcttttattctttgcacaacaacaacaacaacaaattcacggaatcttttaagtgctgaacttatttttcaaccatttcacaaggaggacaagttgaatggacctttttaaaaagaaaaaaaaaatggaaggaaaactaagaatgatcatcttcccagggtgttctcttacttggactgtgatattcgttatttatgaaaaagacttttaaatgccctttctgcagttggaaggttttctttatatactattcccaccatggggagcgaaaacgttaaaatcacaaggaattgcccaatctaagcagactttgccttttttcaaaggtggagcgtgaataccagaaggatccagtattcagtcacttaaatgaagtcttttggtcagaaattacctttttgacacaagcctactgaatgctgtgtatatatttatatataaatatatctatttgagtgaaaccttgtgaactctttaattagagttttcttgtatagtggcagagatgtctatttctgcattcaaaagtgtaatgatgtacttattcatgctaaactttttataaaagtttagttgtaaacttaacccttttatacaaaataaatcaagtgtgtttattgaatggtgattgcctgctttatttcagaggaccagtgctttgatttttattatgctatgttataactgaacccaaataaatacaagttcaaatttatgtagactgtataagattataataaaacatgtctgaagtcaaaaaaaaaaaaaaaaaaaaaaaaaaaaaaaaaaaaaaaa

SEQ ID NO: 13 - DNA, 1252 bp, Homo sapiens
  source 1..1252: mol_type = genomic DNA
gatctggggtgctgccaggaaaaagcaaattctggaagttaatggttttgagtgatttttaaatccttgctggcggagaggcccgcctctccccggtatcagcgcttcctcattctttgaatccgcggctccgcggtcttcggcgtcagaccagccggaggaagcctgtttgcaatttaagcgggctgtgaacgcccagggccggcgggggcagggccgaggcgggccattttgaataaagaggcgtgccttccaggcaggctctataagtgaccgccgcggcgagcgtgcgcgcgttgcaggtcactgtagcgggacttcttttggttttctttctctttggggcacctctggactcactccccagcatgaaggcgctgagcccggtgcgcggctgctacgaggcggtgtgctgcctgtcggaacgcagtctggccatcgcccggggccgagggaagggcccggcagctgaggagccgctgagcttgctggacgacatgaaccactgctactcccgcctgcgggaactggtacccggagtcccgagaggcactcagcttagccaggtggaaatcctacagcgcgtcatcgactacattctcgacctgcaggtagtcctggccgagccagcccctggaccccctgatggcccccaccttcccatccagacagccgagctcactccggaacttgtcatctccaacgacaaaaggagcttttgccactgactcggccgtgtcctgacacctccagaacgcaggtgctggcgcccgttctgcctgggaccccgggaacctctcctgccggaagccggacggcagggatgggccccaacttcgccctgcccacttgacttcaccaaatcccttcctggagactaaacctggtgctcaggagcgaaggactgtgaacttgtggcctgaagagccagagctagctctggccaccagctgggcgacgtcaccctgctcccaccccacccccaagttctaaggtctcttcagagcgtggaggtgtggaaggagtggctgctctccaaactatgccaaggcggcggcagagctggtcttctggtctccttggagaaaggttctgttgccctgatttatgaactctataatagagtatataggttttgtaccttttttacaggaaggtgactttctgtaacaatgcgatgtatattaaactttttataaaagttaacattttgcataataaacgatttttaaacacttgaaaaaaaaaaaaa

SEQ ID NO: 14 - DNA, 3891 bp, Homo sapiens
  source 1..3891: mol_type = genomic DNA
gagagcgtagtggaggaggcgcggttgtgagtagtaccgggagtggggtgatcccgggctaggggagcgcggcggccgcgatcgggcttagtcggagctccgaagggagtgactaggacacccgggtgggctacttttcttccggtgcttttgctttttttttcctttgggctcgggctgagtgtcgcccactgagcaaagattccctcgtaaaacccagagcgaccctcccgtcaattgttgggctcgggagtgtcgcggtgccccgagcgcgccgggcgcggaggcaaagggagcggagccggccgcggacggggcccggagcttgcctgcctccctcgctcgccccagcgggttcgctcgcgtagagcgcagggcgcgcgcgatgaaggcggtgagcccggtgcgcccctcgggccgcaaggcgccgtcgggctgcggcggcggggagctggcgctgcgctgcctggccgagcacggccacagcctgggtggctccgcagccgcggcggcggcggcggcggcagcgcgctgtaaggcggccgaggcggcggccgacgagccggcgctgtgcctgcagtgcgatatgaacgactgctatagccgcctgcggaggctggtgcccaccatcccgcccaacaagaaagtcagcaaagtggagatcctgcagcacgttatcgactacatcctggacctgcagctggcgctggagacgcacccggccctgctgaggcagccaccaccgcccgcgccgccacaccacccggccgggacctgtccagccgcgccgccgcggaccccgctcactgcgctcaacaccgacccggccggcgcggtgaacaagcagggcgacagcattctgtgccgctgagccgcgctgtccaggtgtgcggccgcctgagcccgagccaggagcactagagagggagggggaagagcagaagttagagaaaaaaagccaccggaggaaaggaaaaaacatcggccaacctagaaacgttttcattcgtcattccaagagagagagaggaaagaaaaatacaactttcattctttctttgcacgttcataaacattctacatacgtattctcttttgtctcttcatttataactgctgtgaattgtacatttctgtgttttttggaggtgcagttaaacttttaagcttaagtgtgacaggactgataaatagaagatcaagagtagatccgactttagaagcctactttgtgaccaaggagctcaatttttgttttgaagctttactaatctaccagagcattgtagatattttttttttacatctattgtttaaaatagatgattataacggggcagagaactttcttttctctgcaagaatgttacatattgtatagataaatgagtgacatttcataccatgtatatatagagatgttctataagtgtgagaaagtatatgctttaatagatactgtaattataagatatttttaattaaatatttttttgtaaatattatgtgtgtgtttttttttaatctatgggaatatttcttttggaaaatcatttttcagctcaattacagagctcttgatatcttgaatgtcttttctgtttggcctggctcttaatttgcttttgttttgcccagtatagactcggaagtaacagttatagctagtggtcttgcatgattgcatgagatgtttaatcacaaattaaacttgttctgagtccattcaaatgtgtttttttaaatgtagattgaaatctttgtatttgaagcatacatgttgaaaatacaccttatcagtttttaagtacagggttttatagtgtaatatatacagagtaagtgtttgtttttgtttttcaactgaggtcaaaatggattctgaatgattttgcatatgggatgaggaaatgcttggatccttaaggagtttacgaaatctgctgttttatcaaagtgaaaaaaaattgcttattactcttcattttacactaaagcttaatgtcactaagtttcatgtctgtacagattatttaaatcatggaaatgaaaaaaatgttctctgcttgctaccaaaggacaaactcttggaaatgaacactttctgctttccttcctccaaagaattaataggcaacagtgggagaaaaaaaaggcataatggcaaatccttcaagcagggataaaagtcgatcttcaaacattaacttaagcagaccaaaaattctgatgaccgcatctagattatttttttataaaaatgattttcactatagctatgttacgctaagctactgtccaatctcttgtgatgtgtaacttttacatgtgaatattaaagtagatttctctgtcttgtactgtgatttctggtctcatttctttaaaaccttactcttatttttcttttaaggctcttttttctccttaaggaaggtaatattttctaggttagataggactatcagggtttgtgaacattatgcatttaatgttatgggtactttacacacaagttagatggaatttttagagtgaaagaattaagtaggatttaattgggtgctttgtaaatagtcaactgtgtgtataacgtggtctgtttgatttttaaaaggaaaggatttgtttcagattatacaagaataaaagtattatagacccaagggacttcttatgaggtcaaattcagatatttatatgaatatgaaataccatggtccctagtagtcagttgaagtggcaatgtctaaacagaaatgaacaaaactaatgctagcaggttaaaatcaatcaaaatgtttaaaaattgattctgtcctcagcatgttatttcctcagctctgataatttactggtcttgagtattttgagaatttgatgttgaacgttataaagtcaaagaactgcttgtttagatgaggtttatttttatttttgatattattcattcttgtcacacatcaagaagaaaacactagagtgctgctggaattccaaatctgaagaattctaacgactgcattctttgttattaaaaagggcacaatccttcctttttatttggcagtttaatttcagtaggaagcatgtcacatgtgcactgttggttagaattatgcatctgtcatgcctgactgctgaaccctacctaagccttttggcgcagtttaaaacttatactggtggactgtgaacctcaaaacaaatgggtatttttgggttttgaggatagatgttactccttaaagtttgtatttggggcatgaaaaactactgaaagaagaaaagtgctacagatactacatttcaaagagttggcattttccctttggccactcaagcagcatttgatgtatctaaagaaacaaagtcattgtttattttttaaaaaattatatgcagttgtacaagatactacattccattgaaatgttggctatgtcctaaccaggcaaccagataacaaaaacattttgagtcttttatctaggtagttctaattattcagctacttagtttaacaaaggaaaatatcctgacttctctcatttcatttgtagacttttcattgtataggcacaaccaaagagtcagactggtttaaaactccagaaggaaaaaaagtatcccacacagtggatgttgtttctaagaatgctacaaaatcctgacatctcagacatctcaatgttaaaggaagaaaaaaaataccttttcatttcaaagaactaatatactttgatattgtgtaaaccttactcaagtttattgtcaagctttaactgcctttttagaactttttaaaatttcgagcccacaaatctattgtattagttgccttctataacaataaatcttcactgagcaaaaggcaaaaaaaaaaaaaaa

SEQ ID NO: 15 - DNA, 3104 bp, Homo sapiens
  source 1..3104: mol_type = genomic DNA
ctctgcctgggtgtctccctctctcagtgtgtgtgtctctctgtctgttttcacactctcctccccaatcgagcgaggcccacacctggcgcatcactgccgagccattagctgcgggtttcctttcatcttcgctgtggcagacgtttctatttatccacttgcgctcgccgagtggcgtcaccagcggtactgtaatgacgattgcagcaggaggatgacagcttagaaagaagagggcaatggggcttcctcccagaggcggtgcggcacagaggagcgctcgcttcacaaggtgaccctagctcccaccgccaccgccgcggtcgcggtccagaccgcgctccagcagctccgcgccctcccaggcacccggcctttctttctccctcttgcaaccaagatccgtccggccgctggagacccagggagccggggttaggaactcacttggggctttcccctcccccaccggagagccccgggatggagagccgaaaggacatggttgtgtttctggatgggggtcagcttggcactctggttggcaagagagtctcaaatttgtccgaagccgtgggcagcccgctgccggagccgcccgagaaaatggtgccccgtggttgcctgagccctcgggccgtccctccggccacccgggagcgcggcgggggaggcccggaggaggagccggtagatggactcgcaggcagcgcggcggggccgggcgccgagccccaggtagctggggcggccatgctcggcccaggacccccggccccctcagtcgacagcctctccggacaggggcaacccagtagctcggacaccgagtcggatttctatgaagaaatcgaggtgagctgcaccccggactgcgccaccgggaacgccgagtaccagcacagcaaagggtccggctccgaggcgctggtcggcagtccgaacggagggagcgagacccccaagagcaacggcggcagtggtgggggcggctcgcaaggcaccctggcgtgcagcgccagtgaccagatgcgtcgttaccgcaccgccttcacccgagagcagattgcgcggctggagaaggaattctaccgggagaactacgtatccaggccgcggagatgtgagctggcggccgccctaaacctgccggaaaccaccatcaaggtgtggttccagaaccggcgcatgaaggacaagcggcagcgcctggccatgacgtggccgcacccggcggaccccgccttctacacttacatgatgagccatgcggcggccgcgggcggcctgccctaccccttcccatcgcacctgcccctgccctactactcgccggtgggcctgggcgccgcatccgccgcctccgccgccgcctcgcccttcagcggctcgctgcgcccgctcgacacgttccgcgtgctgtcgcagccctacccgcggcccgaactgctgtgcgccttccgccacccgccgctctaccccgggcccgcgcacggactgggcgcctctgccggcggcccctgctcctgcctcgcctgtcacagcggcccggccaacgggctggcgccccgggctgccgccgcctcggacttcacctgtgcctccacctcccgctcggactccttcctcaccttcgcgccctcggtgctcagcaaggcctcctccgtcgcgctggaccagagggaggaggtgcccctcactagataaggggccgccggctggctgccggctccatgacgcccgtggggtcaccccccggccccgggactcagccagcctcgctcctcgctcctcgctcctcgcccctaggacgccaagggggaaaggagagggcggaaaaggaccagcgggatccggccgcaagaattggaaagcctaggaagtggcggtggctggcgcgtttggggagcaggagtggggatagggaagcagagcttgagagaccttcctccggggcagcctccggacccaccgccccccaccagggtcgaggctgtagctccaaagctaaacaaaacttagcagcaacagcaaccaatatccagtccctcggcccctcggcccctcaccctccacctcacactcccttctcaccgggccccctctccccagccaaggcccaagcactggaaagggaaattgctgtctctctgaacaaaatgctgtgtatgcagagcaggtagagattaatctttgccagcttttccaaggcatgacaaggggctggtggatggcaacataccagtcatttggaggagagagtgagagatgatttactaccagggagaatccagccccttggcatgggacctggagcctcgactacacagcatcttctgggtctggcgtctgccagcacctgatctctttcctcattcccagctttgtgacacttctcaacttgcggctccatctctccctgcccccacttttttgttggccagggaggctgcagatgccccaggagccctttgccgcttctatgaggccaagccttttttccctgggcccagcacacaccctgattagcaagtgatgtgtgcgaggagggtttgtgaatgttgaatgtgtaataatgatcaccatggagctggccactgaccccagagctgagctgttaacaaggcgcccagggaagagcttagggagtgggaacttcacctccctctctcggtatctggcggtaaattagaggcaattttcatcctttgcttgttcaccttcacttcaccaggaactttctggccctaccctttgcattgggtattttacaactttctctcattttcttcccaagctaccactggagcttgactttcagataccagtgggagccttctgtcccttttggggaccctgtctgtggcctccaccagggtttgtttagagccactcccaaatcctcactcccacactcatccttgcagccagtttttgaggaagaggagaacgtgtaaccccaatgcaagcttcaccctgactgagagggagtggttcttcctgtagggaatgaatttggtttgatttggggttttcctttgaagcccaaagaacttgctgttatgattcgttaaccatattgcaataaaagctggacataa

SEQ ID NO: 16 - DNA, 1568 bp, Homo sapiens
  source 1..1568: mol_type = genomic DNA
gagtgaggtttggctgccaccaaagttacttctagtccttgctgtccactcctgccctcagtctggacctgcccaaggacccctgcaattaggcctcccatgcagaggtcagtgagagcccaagccaattgctctaggccccgtggctggctacttatggggcactgtcctgaccagctctgctaagatgctcctggcccctccctccaccccgtccagaggacggacccccagcgccgtggagaggctggaagccgacaaagccaagtatgtcaagacgcaccaggtgatagctaggcgacaggagccagccctgcgtgggagtcctgggccgctcacgccgcacccctgcaacgagctggggccccctgcatcgcccaggacgcccaggccggtccgccggggaagcggcaggcggctgccgaggcctgattccctcatcttctaccgccagaagcgggactgcaaggcttcggtgaacaaagagaacgccaagggccagggtctggtgcggcgcctctttctgggtgccccgcgggacgctgccccgagcagcccggcctccacagagcgacctgcggcttcagggggttgggctgcgccccaggatgccccggaagcggcgggaaagcgggcgctgtgtcccacgtgctcgctgcccctgtcggagaaggagcgcttcttcaactactgcggcctggagcgcgcgctggtggaggtgctgggcgcagagcgcttctccccgcagagctggggagccgacgccagcccgcaggccggaacttcgccgccgcccggctccggggacgccagcgactggacatccagcgacaggggcgtggacagcccgggcggcgcgggcggcggcggcggctcggaggcagcgggctcggcgcgggaccggcgccccccggtgtcggtggtggagcgcaacgcgcgcgtcatccagtggctgtacggctgccagcgcgcccgcggaccgccgcgcgagtccgaggtgtgaccgccgcggctccggactggccccgggactggccccgggcacggaaaaggacacccctcttctggcgcgctgggtgcctttgcgtaagcccttccttctggaactcagtttcgcgtctgaaccttggggaggtggaacaagttgctgccgaaggcccttccctgctcccgcggcgaagggggagggagaggcctcttggtccctgtggagacccggtctggggagtcacgattggggtgggagatgagcaaacctgctgaataaagttaaaacgttatttaaatggggagctgaggaaggagcaaacgggttttcgcggttaaacccgtgggttttggaatgtgtgttcccggctgtgtgatcctgggcaagaacttgacctccctggacgcagcggcacccctcggttattaaggagggaggagtaggggatagaagtatttcaaaatagttgtaatgcgcatggcaaagtgcccagcatatagaaagtgctcaataaacgataactgctgtgacttctaaaaaaaaaaaaaaaaaaaaaaaaaaaaaaaaaaaaaaaaaaaaa

SEQ ID NO: 17 - DNA, 1184 bp, Homo sapiens
  source 1..1184: mol_type = genomic DNA
tggaaggggccacttcacacctcgggctcggcataaagcggccgccggccgccggcccccagacgcgccgccgctgccatggcccagcccctgtgcccgccgctctccgagtcctggatgctctctgcggcctggggcccaactcggcggccgccgccctccgacaaggactgcggccgctccctcgtctcgtccccagactcatggggcagcaccccagccgacagccccgtggcgagccccgcgcggccaggcaccctccgggacccccgcgccccctccgtaggtaggcgcggcgcgcgcagcagccgcctgggcagcgggcagaggcagagcgccagtgagcgggagaaactgcgcatgcgcacgctggcccgcgccctgcacgagctgcgccgctttctaccgccgtccgtggcgcccgcgggccagagcctgaccaagatcgagacgctgcgcctggctatccgctatatcggccacctgtcggccgtgctaggcctcagcgaggagagtctccagcgccggtgccggcagcgcggtgacgcggggtcccctcggggctgcccgctgtgccccgacgactgccccgcgcagatgcagacacggacgcaggctgaggggcaggggcaggggcgcgggctgggcctggtatccgccgtccgcgccggggcgtcctggggatccccgcctgcctgccccggagcccgagctgcacccgagccgcgcgacccgcctgcgctgttcgccgaggcggcgtgccctgaagggcaggcgatggagccaagcccaccgtccccgctccttccgggcgacgtgctggctctgttggagacctggatgcccctctcgcctctggagtggctgcctgaggagcccaagtgacaagggacaactgacgccgtctctgtgagcaccgaggctttttggcctcagcaccttcgaagtggttccttggcagactgcctttcctggaagagggcacgggcgatcccgacgggggcattcctgcgggtgagagccgtccccaccgcggcggcccttctcagcccctccctccatggagggacccatagggctagacactttgaggcaagcaggaggctctgcctaatgtgaatttatttatttgtgaataaactgtactggtgtcagttggcaaaaaaaaaaaaaaaaaaaaaaaa

SEQ ID NO: 18 - DNA, 2428 bp, Homo sapiens
  source 1..2428: mol_type = genomic DNA
cccgcccacttccaactaccgcctccggcctgcccagggagagagagggagtggagcccagggagagggagcgcgagagagggagggaggaggggacggtgctttggctgacttttttttaaaagagggtgggggtggggggtgattgctggtcgtttgttgtggctgttaaattttaaactgccatgcactcggcttccagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggttttatcccttgaatcttttcttccagatttttcttttctttacttggcttacaaaatatacaggcttggaaattatttcaagaaggagggagggataccctgtctggttgcaggttgtattttattttggcccagggagtgttgctgttttcccaacattttattaataaaattttcagacataaaaaa

SEQ ID NO: 19 - DNA, 4084 bp, Homo sapiens
  source 1..4084: mol_type = genomic DNA
ggtttccaggcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcactgggcgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcagtacggacgaggtgctgtccctggaggagaaagacctgagggaccgggagaggcgcatggccaataacgcgcgggagcgggtgcgcgtgcgggatattaacgaggccttccgggagctggggcgcatgtgccagatgcacctcaagtcggacaaagcgcagaccaagctgctcatcctgcagcaggccgtgcaggtcatcctggggctggagcagcaggtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaatgtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagcccccccgtccccccccccccccggaagccactgcacacagacagaacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggcccccagggctaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactcctttgccccagggacccaggctcatgctctgaactctggctgagaggatgctgctcaggagccagcacaggacaccccccaccccaccccaccatgtccccattacaccagagggccatcgtgacgtagacaggatgccaggggcctggccagcctcccccaatgctggggagcatccctgggcctggggccacacctgctgccctccctctgtgtggtccaagggcaagagtggctggagccgggggactgtgctggtctgagccccacgaaggccttgggctgtgcgtccgaccctgctgcagaaccagcagggtgtcccctcgggcccatctgtgtcccatgtcccagcacccaggcctctctccaggtctccttttctggtcttttgccatgagggtaaccagctcttcccagctggctggggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactcccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctacgccctgggagtttgatctcttaagggaacttgcctctccctcttgttttgctcctggccctgcccctaggtctgggtgggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaaatttttttaaagaaaataattgtatacgtgtcaacacagctggctggatgattgggactttaaaacgaccctctttcaggtggattcagagacctgtcctgtatataacagcactgtagcaataaacgtgacattttataacgatgccctgca

SEQ ID NO: 20 - DNA, 1000 bp, Homo sapiens
  source 1..1000: mol_type = genomic DNA
actctcattccacgttcttaactgttccattttccgtatctgcttcgggcttccacctcatttttttcgctttgcccattctgtttcagccagtcgccaagaatcatgaaagtcgccagtggcagcaccgccaccgccgccgcgggccccagctgcgcgctgaaggccggcaagacagcgagcggtgcgggcgaggtggtgcgctgtctgtctgagcagagcgtggccatctcgcgctgcgccgggggcgccggggcgcgcctgcctgccctgctggacgagcagcaggtaaacgtgctgctctacgacatgaacggctgttactcacgcctcaaggagctggtgcccaccctgccccagaaccgcaaggtgagcaaggtggagattctccagcacgtcatcgactacatcagggaccttcagttggagctgaactcggaatccgaagttggaacccccgggggccgagggctgccggtccgggctccgctcagcaccctcaacggcgagatcagcgccctgacggccgaggcggcatgcgttcctgcggacgatcgcatcttgtgtcgctgaagcgcctcccccagggaccggcggaccccagccatccagggggcaagaggaattacgtgctctgtgggtctcccccaacgcgcctcgccggatctgagggagaacaagaccgatcggcggccactgcgcccttaactgcatccagcctggggctgaggctgaggcactggcgaggagagggcgctcctctctgcacacctactagtcaccagagactttagggggtgggattccactcgtgtgtttctattttttgaaaagcagacattttaaaaaatggtcacgtttggtgcttctcagatttctgaggaaattgctttgtattgtatattacaatgatcaccgactgaaaatattgttttacaatagttctgtggggctgtttttttgttattaaacaaataatttagatggtggtaaaaaaaaa

SEQ ID NO: 21 - DNA, 2415 bp, Homo sapiens
  source 1..2415: mol_type = genomic DNA
cggccgctgctagaggggctgcttgcgccaggcgccggccgccccactgcgggtccctggcggccggtgtctgaggagtcggagagccgaggcggccagaccgtgcgccccgcgcttctcccgaggccgttccgggtctgaactgtaacagggaggggcctcgcaggagcagcagcgggcgagttaaagtatgctgggagcggtgaagatggaagggcacgagccgtccgactggagcagctactatgcagagcccgagggctactcctccgtgagcaacatgaacgccggcctggggatgaacggcatgaacacgtacatgagcatgtcggcggccgccatgggcagcggctcgggcaacatgagcgcgggctccatgaacatgtcgtcgtacgtgggcgctggcatgagcccgtccctggcggggatgtcccccggcgcgggcgccatggcgggcatgggcggctcggccggggcggccggcgtggcgggcatggggccgcacttgagtcccagcctgagcccgctcggggggcaggcggccggggccatgggcggcctggccccctacgccaacatgaactccatgagccccatgtacgggcaggcgggcctgagccgcgcccgcgaccccaagacctacaggcgcagctacacgcacgcaaagccgccctactcgtacatctcgctcatcaccatggccatccagcagagccccaacaagatgctgacgctgagcgagatctaccagtggatcatggacctcttccccttctaccggcagaaccagcagcgctggcagaactccatccgccactcgctctccttcaacgactgtttcctgaaggtgccccgctcgcccgacaagcccggcaagggctccttctggaccctgcaccctgactcgggcaacatgttcgagaacggctgctacctgcgccgccagaagcgcttcaagtgcgagaagcagctggcgctgaaggaggccgcaggcgccgccggcagcggcaagaaggcggccgccggagcccaggcctcacaggctcaactcggggaggccgccgggccggcctccgagactccggcgggcaccgagtcgcctcactcgagcgcctccccgtgccaggagcacaagcgagggggcctgggagagctgaaggggacgccggctgcggcgctgagccccccagagccggcgccctctcccgggcagcagcagcaggccgcggcccacctgctgggcccgccccaccacccgggcctgccgcctgaggcccacctgaagccggaacaccactacgccttcaaccacccgttctccatcaacaacctcatgtcctcggagcagcagcaccaccacagccaccaccaccaccaaccccacaaaatggacctcaaggcctacgaacaggtgatgcactaccccggctacggttcccccatgcctggcagcttggccatgggcccggtcacgaacaaaacgggcctggacgcctcgcccctggccgcagatacctcctactaccagggggtgtactcccggcccattatgaactcctcttaagaagacgacggcttcaggcccggctaactctggcaccccggatcgaggacaagtgagagagcaagtgggggtcgagactttggggagacggtgttgcagagacgcaagggagaagaaatccataacacccccaccccaacacccccaagacagcagtcttcttcacccgctgcagccgttccgtcccaaacagagggccacacagataccccacgttctatataaggaggaaaacgggaaagaatataaagttaaaaaaaagcctccggtttccactactgtgtagactcctgcttcttcaagcacctgcagattctgatttttttgttgttgttgttctcctccattgctgttgttgcagggaagtcttacttaaaaaaaaaaaaaaattttgtgagtgactcggtgtaaaaccatgtagttttaacagaaccagagggttgtactattgtttaaaaacaggaaaaaaaataatgtaagggtctgttgtaaatgaccaagaaaaagaaaaaaaaagcattcccaatcttgacacggtgaaatccaggtctcgggtccgattaatttatggtttctgcgtgctttatttatggcttataaatgtgtattctggctgcaagggccagagttccacaaatctatattaaagtgttatacccggttttatcccttgaatcttttcttccagatttttcttttctttacttggcttacaaaatatacaggcttggaaattatttcaagaaggagggagggataccctgtctggttgcaggttgtattttattttggcccagggagtgttgctgttttcccaacattttattaataaaattttcagacataaaaaa

SEQ ID NO: 22 - DNA, 2350 bp, Homo sapiens
  source 1..2350: mol_type = genomic DNA
acgcgccgcgtgcccggccgcgcccagcagggtttccaggcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgagtaagatagaagaccacctggacgaggccatccacgtgctccgcagccacgccgtgggcacagccggcgacatgcacacgctgctgcctggccacggggcgctggcctcaggtttcaccggccccatgtcactgggcgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtctgggtgggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaaatttttttaaagaaaataattgtatacgtgtcaacacagctggctggatgattgggactttaaaacgaccctctttcaggtggattcagagacctgtcctgtatataacagcactgtagcaataaacgtgacattttataacgatgccctgca

SEQ ID NO: 23 - DNA, 4448 bp, Homo sapiens
  source 1..4448: mol_type = genomic DNA
acgcgccgcgtgcccggccgcgcccagcagggtttccaggcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcactgggcgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcagtacggacgaggtgctgtccctggaggagaaagacctgagggaccgggagaggcgcatggccaataacgcgcgggagcgggtgcgcgtgcgggatattaacgaggccttccgggagctggggcgcatgtgccagatgcacctcaagtcggacaaagcgcagaccaagctgctcatcctgcagcaggccgtgcaggtcatcctggggctggagcagcaggtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtatgcctccgtgggacgagccacccgctttcagccctgtgctctggccccagaacggccactcgagaccccgggcttcatccacatccacacctcacacacctgttgtcagcatcgagccaacaccaacctgacaaggttcggagtgatgggggcggccaaggtgacactgggtccaggagctccctggggccctggcctaccactcactggcctcgctccccctgtccccgaatctcagccaccgtgtcactctgtgacctgtcccatggatcctgaaactgcatcttggccctgttgcctgggctgacaggagcattttttttttttccagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaatgtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagcccccccgtccccccccccccccggaagccactgcacacagacagaacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggcccccagggctaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactcctttgccccagggacccaggctcatgctctgaactctggctgagaggatgctgctcaggagccagcacaggacaccccccaccccaccccaccatgtccccattacaccagagggccatcgtgacgtagacaggatgccaggggcctggccagcctcccccaatgctggggagcatccctgggcctggggccacacctgctgccctccctctgtgtggtccaagggcaagagtggctggagccgggggactgtgctggtctgagccccacgaaggccttgggctgtgcgtccgaccctgctgcagaaccagcagggtgtcccctcgggcccatctgtgtcccatgtcccagcacccaggcctctctccaggtctccttttctggtcttttgccatgagggtaaccagctcttcccagctggctggggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactcccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctacgccctgggagtttgatctcttaagggaacttgcctctccctcttgttttgctcctggccctgcccctaggtctgggtgggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaaatttttttaaagaaaataattgtatacgtgtcaacacagctggctggatgattgggactttaaaacgaccctctttcaggtggattcagagacctgtcctgtatataacagcactgtagcaataaacgtgacattttataacgatgccctgca

SEQ ID NO: 24 - DNA, 4457 bp, Homo sapiens
  source 1..4457: mol_type = genomic DNA
acgcgccgcgtgcccggccgcgcccagcagggtttccaggcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcactgggcgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcccagacgaggacgaggacgaccttctccccccagagcagaaggccgagcgggagaaggagcgccgggtggccaataacgcccgggagcggctgcgggtccgtgacatcaacgaggcctttaaggagctggggcgcatgtgccaactgcacctcaacagcgagaagccccagaccaaactgctcatcctgcaccaggctgtctcggtcatcctgaacttggagcagcaagtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaaggtatgcctccgtgggacgagccacccgctttcagccctgtgctctggccccagaacggccactcgagaccccgggcttcatccacatccacacctcacacacctgttgtcagcatcgagccaacaccaacctgacaaggttcggagtgatgggggcggccaaggtgacactgggtccaggagctccctggggccctggcctaccactcactggcctcgctccccctgtccccgaatctcagccaccgtgtcactctgtgacctgtcccatggatcctgaaactgcatcttggccctgttgcctgggctgacaggagcattttttttttttccagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaatgtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagcccccccgtccccccccccccccggaagccactgcacacagacagaacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggcccccagggctaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactcctttgccccagggacccaggctcatgctctgaactctggctgagaggatgctgctcaggagccagcacaggacaccccccaccccaccccaccatgtccccattacaccagagggccatcgtgacgtagacaggatgccaggggcctggccagcctcccccaatgctggggagcatccctgggcctggggccacacctgctgccctccctctgtgtggtccaagggcaagagtggctggagccgggggactgtgctggtctgagccccacgaaggccttgggctgtgcgtccgaccctgctgcagaaccagcagggtgtcccctcgggcccatctgtgtcccatgtcccagcacccaggcctctctccaggtctccttttctggtcttttgccatgagggtaaccagctcttcccagctggctggggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactcccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctacgccctgggagtttgatctcttaagggaacttgcctctccctcttgttttgctcctggccctgcccctaggtctgggtgggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaaatttttttaaagaaaataattgtatacgtgtcaacacagctggctggatgattgggactttaaaacgaccctctttcaggtggattcagagacctgtcctgtatataacagcactgtagcaataaacgtgacattttataacgatgccctgca

SEQ ID NO: 25 - DNA, 21 bp, synthetic construct
  source 1..21: mol_type = other DNA
ttcttcccccttgttgtcctc

SEQ ID NO: 26 - DNA, 22 bp, synthetic construct
  source 1..22: mol_type = other DNA
acaggtgtagttggtctgtagg

SEQ ID NO: 27 - DNA, 21 bp, synthetic construct
  source 1..21: mol_type = other DNA
ctggattctggcgatggtgta

SEQ ID NO: 28 - DNA, 21 bp, synthetic construct
  source 1..21: mol_type = other DNA
cggacaatttcacgttcagca

SEQ ID NO: 29 - DNA, 21 bp, synthetic construct
  source 1..21: mol_type = other DNA
aacagcagcggaaaaccttca

SEQ ID NO: 30 - DNA, 23 bp, synthetic construct
  source 1..23: mol_type = other DNA
ggctttattgacattggcgattt

SEQ ID NO: 31 - DNA, 22 bp, synthetic construct
  source 1..22: mol_type = other DNA
ctgggtctggaaccaattcttt

SEQ ID NO: 32 - DNA, 20 bp, synthetic construct
  source 1..20: mol_type = other DNA
gcctgagccatcagtgtgta

SEQ ID NO: 33 - DNA, 19 bp, synthetic construct
  source 1..19: mol_type = other DNA
cactgctttgggagccttc

SEQ ID NO: 34 - DNA, 21 bp, synthetic construct
  source 1..21: mol_type = other DNA
ggggcagcgattcatttttct

SEQ ID NO: 35 - DNA, 21 bp, synthetic construct
  source 1..21: mol_type = other DNA
ttccccgaagacacacacaaa

SEQ ID NO: 36 - DNA, 21 bp, synthetic construct
  source 1..21: mol_type = other DNA
ccccagttttcattgccctca

SEQ ID NO: 37 - DNA, 22 bp, synthetic construct
  source 1..22: mol_type = other DNA
atgcctaacttttctggcaact

SEQ ID NO: 38 - DNA, 21 bp, synthetic construct
  source 1..21: mol_type = other DNA
gcacagtggtggaggttttga

SEQ ID NO: 39 - DNA, 21 bp, synthetic construct
  source 1..21: mol_type = other DNA
tgcatcagtgacggtaaacca

SEQ ID NO: 40 - DNA, 21 bp, synthetic construct
  source 1..21: mol_type = other DNA
ttcttcagccgtgcaacaatc

SEQ ID NO: 41 - DNA, 23 bp, synthetic construct
  source 1..23: mol_type = other DNA
tgaaaataaaggtgcctcgtctc

SEQ ID NO: 42 - DNA, 22 bp, synthetic construct
  source 1..22: mol_type = other DNA
tcaggttactgaatagcctccc

SEQ ID NO: 43 - DNA, 20 bp, synthetic construct
  source 1..20: mol_type = other DNA
tcggctcctttcctcactca

SEQ ID NO: 44 - DNA, 21 bp, synthetic construct
  source 1..21: mol_type = other DNA
ctcatagggttgttcgctcgg

SEQ ID NO: 45 - DNA, 21 bp, synthetic construct
  source 1..21: mol_type = other DNA
tcctatgcacgtcccagttct

SEQ ID NO: 46 - DNA, 21 bp, synthetic construct
  source 1..21: mol_type = other DNA
tcgatcctcttgtcaaagcgg

SEQ ID NO: 47 - DNA, 21 bp, synthetic construct
  source 1..21: mol_type = other DNA
gagagccgaaaggacatggtt

SEQ ID NO: 48 - DNA, 20 bp, synthetic construct
  source 1..20: mol_type = other DNA
ctgcctgctagtccatcgac

SEQ ID NO: 49 - DNA, 21 bp, synthetic construct
  source 1..21: mol_type = other DNA
ccgaggagggatctaaggaac

SEQ ID NO: 50 - DNA, 23 bp, synthetic construct
  source 1..23: mol_type = other DNA
cttccaaaagtatcggtctccac

SEQ ID NO: 51 - DNA, 19 bp, synthetic construct
  source 1..19: mol_type = other DNA
ggtcgcttcgtgagtccag

SEQ ID NO: 52 - DNA, 19 bp, synthetic construct
  source 1..19: mol_type = other DNA
agcagccgtctccagtagt

SEQ ID NO: 53 - DNA, 21 bp, synthetic construct
  source 1..21: mol_type = other DNA
ccctacgccaacatgaactcg

SEQ ID NO: 54 - DNA, 20 bp, synthetic construct
  source 1..20: mol_type = other DNA
gttctgccggtagaaaggga

SEQ ID NO: 55 - DNA, 20 bp, synthetic construct
  source 1..20: mol_type = other DNA
gggaaagcactgcacgaact

SEQ ID NO: 56 - DNA, 21 bp, synthetic construct
  source 1..21: mol_type = other DNA
agcacgcaaaaggtcacattg

SEQ ID NO: 57 - DNA, 20 bp, synthetic construct
  source 1..20: mol_type = other DNA
caacttcgacaaagccgagg

SEQ ID NO: 58 - DNA, 20 bp, synthetic construct
  source 1..20: mol_type = other DNA
actcgtctttcccttgccct

SEQ ID NO: 59 - DNA, 22 bp, synthetic construct
  source 1..22: mol_type = other DNA
gccaacattgtcctcgtaaact

SEQ ID NO: 60 - DNA, 21 bp, synthetic construct
  source 1..21: mol_type = other DNA
tgtggtccccttcacttatgg

SEQ ID NO: 61 - DNA, 21 bp, synthetic construct
  source 1..21: mol_type = other DNA
gcagaaacatctggtttgctg

SEQ ID NO: 62 - DNA, 22 bp, synthetic construct
  source 1..22: mol_type = other DNA
cctcctgttcataggtggagtc

SEQ ID NO: 63 - DNA, 19 bp, synthetic construct
  source 1..19: mol_type = other DNA
agggaccactgcaactcag

SEQ ID NO: 64 - DNA, 22 bp, synthetic construct
  source 1..22: mol_type = other DNA
ccatacacagttaaggacgcac

SEQ ID NO: 65 - DNA, 21 bp, synthetic construct
  source 1..21: mol_type = other DNA
cagatgctgccctacatgaac

SEQ ID NO: 66 - DNA, 21 bp, synthetic construct
  source 1..21: mol_type = other DNA
tctgggtacttcgtctcctgg

SEQ ID NO: 67 - DNA, 20 bp, synthetic construct
  source 1..20: mol_type = other DNA
cctagctgttcgctgaaggc

SEQ ID NO: 68 - DNA, 21 bp, synthetic construct
  source 1..21: mol_type = other DNA
ctccgacagaccaagtaccac

SEQ ID NO: 69 - DNA, 19 bp, synthetic construct
  source 1..19: mol_type = other DNA
cgaccgaggagcctcttag

SEQ ID NO: 70 - DNA, 19 bp, synthetic construct
  source 1..19: mol_type = other DNA
ggacgcgatagggaagacc

SEQ ID NO: 71 - DNA, 21 bp, synthetic construct
  source 1..21: mol_type = other DNA
tctgggtccctatccaatgtg

SEQ ID NO: 72 - DNA, 19 bp, synthetic construct
  source 1..19: mol_type = other DNA
ggtccccgaactggtactg

SEQ ID NO: 73 - DNA, 21 bp, synthetic construct
  source 1..21: mol_type = other DNA
tacagcaccagcgtcatttcg

SEQ ID NO: 74 - DNA, 20 bp, synthetic construct
  source 1..20: mol_type = other DNA
gagcccacgtaagagaaggc

SEQ ID NO: 75 - DNA, 23 bp, synthetic construct
  source 1..23: mol_type = other DNA
tttggcaaatacaacccttcaga

SEQ ID NO: 76 - DNA, 21 bp, synthetic construct
  source 1..21: mol_type = other DNA
gcagaagatactgtcaccacc

SEQ ID NO: 77 - DNA, 19 bp, synthetic construct
  source 1..19: mol_type = other DNA
ccaaccgcactgcccttat

SEQ ID NO: 78 - DNA, 20 bp, synthetic construct
  source 1..20: mol_type = other DNA
cgcgaaacgaaccaacttgt

SEQ ID NO: 79 - DNA, 21 bp, synthetic construct
  source 1..21: mol_type = other DNA
cagccagaattttcgagaggt

SEQ ID NO: 80 - DNA, 19 bp, synthetic construct
  source 1..19: mol_type = other DNA
cagtgcgattggagccatc

SEQ ID NO: 81 - DNA, 21 bp, synthetic construct
  source 1..21: mol_type = other DNA
gtcactcggtcctggtttaag

SEQ ID NO: 82 - DNA, 20 bp, synthetic construct
  source 1..20: mol_type = other DNA
acgatgggtcccacgattct

SEQ ID NO: 83 - DNA, 20 bp, synthetic construct
  source 1..20: mol_type = other DNA
gcccagtacctccgaaagtc

SEQ ID NO: 84 - DNA, 23 bp, synthetic construct
  source 1..23: mol_type = other DNA
gccttaacatactcctccttgtc

SEQ ID NO: 85 - DNA, 23 bp, synthetic construct
  source 1..23: mol_type = other DNA
gagacatccccctatttctacca

SEQ ID NO: 86 - DNA, 19 bp, synthetic construct
  source 1..19: mol_type = other DNA
gctcagtccgctcatagcc

SEQ ID NO: 87 - DNA, 21 bp, synthetic construct
  source 1..21: mol_type = other DNA
ttcaagcctgttgggctctac

SEQ ID NO: 88 - DNA, 20 bp, synthetic construct
  source 1..20: mol_type = other DNA
tccggtcacgtccacatctt

SEQ ID NO: 89 - DNA, 20 bp, synthetic construct
  source 1..20: mol_type = other DNA
acgctggtgctctatgcaag

SEQ ID NO: 90 - DNA, 21 bp, synthetic construct
  source 1..21: mol_type = other DNA
tcagttgctgcccattcatca

SEQ ID NO: 91 - DNA, 21 bp, synthetic construct
  source 1..21: mol_type = other DNA
gcagccgttgaatgtctcttc

SEQ ID NO: 92 - DNA, 21 bp, synthetic construct
  source 1..21: mol_type = other DNA
gtccgtgaactcgaccttttt

SEQ ID NO: 93 - DNA, 19 bp, synthetic construct
  source 1..19: mol_type = other DNA
cacacgctgccttgtgtct

SEQ ID NO: 94 - DNA, 19 bp, synthetic construct
  source 1..19: mol_type = other DNA
ggtcagcaaaagcacggtt

SEQ ID NO: 95 - DNA, 20 bp, synthetic construct
  source 1..20: mol_type = other DNA
gagccggatctgaagaggga

SEQ ID NO: 96 - DNA, 20 bp, synthetic construct
  source 1..20: mol_type = other DNA
gcttgacgtgtggcttgttc

SEQ ID NO: 97 - DNA, 21 bp, synthetic construct
  source 1..21: mol_type = other DNA
tcattcggctacggaacaaga

SEQ ID NO: 98 - DNA, 21 bp, synthetic construct
  source 1..21: mol_type = other DNA
gacctgcgactccaaagtctg

SEQ ID NO: 99 - DNA, 21 bp, synthetic construct
  source 1..21: mol_type = other DNA
cagaggaggccaacgtagaag

SEQ ID NO: 100 - DNA, 20 bp, synthetic construct
  source 1..20: mol_type = other DNA
ctccatcggggatcttgggt

SEQ ID NO: 101 - DNA, 21 bp, synthetic construct
  source 1..21: mol_type = other DNA
atgctcctgaaagcaaaccag

SEQ ID NO: 102 - DNA, 22 bp, synthetic construct
  source 1..22: mol_type = other DNA
ccttttagggcaagtccattgt

SEQ ID NO: 103 - DNA, 19 bp, synthetic construct
  source 1..19: mol_type = other DNA
ccactctctgcgagcaatg

SEQ ID NO: 104 - DNA, 19 bp, synthetic construct
  source 1..19: mol_type = other DNA
ggtggctgaactagccgat

SEQ ID NO: 105 - DNA, 22 bp, synthetic construct
  source 1..22: mol_type = other DNA
atgagtctcctaaaggaacgga

SEQ ID NO: 106 - DNA, 22 bp, synthetic construct
  source 1..22: mol_type = other DNA
gggaatacgatcttgctctgac

SEQ ID NO: 107 - DNA, 19 bp, synthetic construct
  source 1..19: mol_type = other DNA
gcatcggtggacgctatgt

SEQ ID NO: 108 - DNA, 21 bp, synthetic construct
  source 1..21: mol_type = other DNA
tgtgctaggaaggtctcccaa

SEQ ID NO: 109 - DNA, 21 bp, synthetic construct
  source 1..21: mol_type = other DNA
gatgaccccatcggaaacctg

SEQ ID NO: 110 - DNA, 20 bp, synthetic construct
  source 1..20: mol_type = other DNA
cttgcagatcctgttggcag

SEQ ID NO: 111 - DNA, 21 bp, synthetic construct
  source 1..21: mol_type = other DNA
gtatcctcgaaggacaaccct

SEQ ID NO: 112 - DNA, 21 bp, synthetic construct
  source 1..21: mol_type = other DNA
gacatcggtcagtgtgatcgt

SEQ ID NO: 113 - DNA, 20 bp, synthetic construct
  source 1..20: mol_type = other DNA
aagcgtgagtcgcaagaatg

SEQ ID NO: 114 - DNA, 20 bp, synthetic construct
  source 1..20: mol_type = other DNA
tctccaggttttcgccagtg

SEQ ID NO: 115 - DNA, 20 bp, synthetic construct
  source 1..20: mol_type = other DNA
gaccagatgcgtcgttaccg

SEQ ID NO: 116 - DNA, 20 bp, synthetic construct
  source 1..20: mol_type = other DNA
gtggtttccggcaggtttag

SEQ ID NO: 117 - DNA, 21 bp, synthetic construct
  source 1..21: mol_type = other DNA
tcaagacgcaccaggtgatag

SEQ ID NO: 118 - DNA, 23 bp, synthetic construct
  source 1..23: mol_type = other DNA
cggtagaagatgagggaatcagg

SEQ ID NO: 119 - DNA, 20 bp, synthetic construct
  source 1..20: mol_type = other DNA
ctgctctacgacatgaacgg

SEQ ID NO: 120 - DNA, 22 bp, synthetic construct
  source 1..22: mol_type = other DNA
gaaggtccctgatgtagtcgat

SEQ ID NO: 121 - DNA, 22 bp, synthetic construct
  source 1..22: mol_type = other DNA
gtgatcggaaatgacactggag

SEQ ID NO: 122 - DNA, 23 bp, synthetic construct
  source 1..23: mol_type = other DNA
catgttggtcactaacagaagca

SEQ ID NO: 123 - DNA, 20 bp, synthetic construct
  source 1..20: mol_type = other DNA
ccaccgtccccgctccttcc

SEQ ID NO: 124 - DNA, 20 bp, synthetic construct
  source 1..20: mol_type = other DNA
cggtgctcacagagacggcg

SEQ ID NO: 125 - DNA, 21 bp, synthetic construct
  source 1..21: mol_type = other DNA
gctggtcaccaacaatcccta

SEQ ID NO: 126 - DNA, 21 bp, synthetic construct
  source 1..21: mol_type = other DNA
cgtcaaaggcactatcggtgg

SEQ ID NO: 127 - DNA, 21 bp, synthetic construct
  source 1..21: mol_type = other DNA
ggggaaaactacctgcctgtc

SEQ ID NO: 128 - DNA, 20 bp, synthetic construct
  source 1..20: mol_type = other DNA
aggcgctcgatgtactggat

SEQ ID NO: 129 - DNA, 19 bp, synthetic construct
  source 1..19: mol_type = other DNA
ccaaggtgggatcgtgagg

SEQ ID NO: 130 - DNA, 21 bp, synthetic construct
  source 1..21: mol_type = other DNA
tcggaaggataaaacgcggtc

SEQ ID NO: 131 - DNA, 21 bp, synthetic construct
  source 1..21: mol_type = other DNA
tcggaagcctaactacagcga

SEQ ID NO: 132 - DNA, 20 bp, synthetic construct
  source 1..20: mol_type = other DNA
agatgagcattggcagcgag

SEQ ID NO: 133 - DNA, 21 bp, synthetic construct
  source 1..21: mol_type = other DNA
acagagcggaaaagtgggaag

SEQ ID NO: 134 - DNA, 21 bp, synthetic construct
  source 1..21: mol_type = other DNA
tcgttgatcctgtttcggaga

SEQ ID NO: 135 - protein, 141 aa, Homo sapiens
PSCALKAGKTASGAGEVVRCLSEQSVAISRCAGGAGARLPALLDEQQVNVLLYDMNGCYSRLKELVPTLPQNRKVSKVEILQHVIDYIRDLQLELNSESEVGTPGGRGLPVRAPLSTLNGEISALTAEAACVPADDRILCR

SEQ ID NO: 136 - protein, 105 aa, Xenopus laevis
SLTEHSLGIARSKTPVDDPMSLLYNMNDCYSKLKELVPSIPQNKKVSKMEILQHVIDYILDLQLTLDSHPSIVSLHHLPRVGGNTSRTPLDPLNTDISILSLQAA

SEQ ID NO: 137 - protein, 108 aa, Mus musculus
GLSEQSVAISRCAGTRLPALLDEQQVNVLLYDMNGCYSRLKELVPTLPQNRKVSKVEILQHVIDYIRDLQLELNSESEVGTTGGRGLPVRAPLSTLNGEISALAAEAA

(19) United States
(12) Patent Application Publication
(10) Pub. No.: US 2024/0110156 A1
(43) Pub. Date: Apr. 4, 2024

(54) CARDIOGENIC MESODERM FORMATION REGULATORS

(71) Applicant: Sanford Burnham Prebys Medical Discovery Institute, La Jolla, CA (US)

(72) Inventors: Alexandre Romain Colas, La Jolla, CA (US); Mark Mercola, Los Altos, CA (US); Wesley Lawrence McKeithan, Palo Alto, CA (US); Michael Shenghan Yu, San Diego, CA (US)

(21) Appl. No.: 18/474,821

(22) Filed: Sep. 26, 2023

Related U.S. Application Data
(62) Division of application No. 16/638,918, filed on Feb. 13, 2020, now Pat. No. 11,821,003.
(60) Provisional application No. 62/545,310, filed on Aug. 14, 2017.

(51) Int. Cl. C12N 5/077; A61P 9/00; C07K 14/47

(52) U.S. Cl. CPC C12N 5/0657; A61P 9/00; C07K 14/4702; C12N 2506/02; C12N 2506/45

(57) ABSTRACT

This disclosure relates to cardiogenic mesoderm formation regulators and methods of use thereof, e.g., generating a multipotent cardiovascular progenitor cell by overexpressing Id1, Id2, Id3, Id4, Evx1, and/or Grrp1 in a stem cell.

### CROSS-REFERENCE TO RELATED APPLICATIONS

Any and all priority claims identified in the Application Data sheet, or any correction thereto, are hereby incorporated by reference under 37 CFR 1.57. For example, this application is a divisional of U.S. patent application Ser. No. 16/638,918, filed Feb. 13, 2020, which is a National Stage Entry of PCT/US2018/046536, filed Aug. 13, 2018, which claims priority from 62/545,310, filed Aug. 14, 2017, all of which are incorporated herein by reference in their entireties.

### FEDERALLY SPONSORED RESEARCH OR DEVELOPMENT

This invention was made with government support under R01 AR052779, P30 AR061303, R33 HL088266, R01 AR056712, F31 AR065923, P30 CA030199, and R01 HL113601 awarded by the National Institutes of Health. The government has certain rights in the invention.

### SEQUENCE LISTING

This application contains a Sequence Listing that has been submitted electronically as an XML file named 43820-0004002 SL ST26.xml. The XML file, created on Sep. 20, 2023, is 158,947 bytes in size. The material in the XML file is hereby incorporated by reference in its entirety.

### TECHNICAL FIELD

This disclosure relates to cardiogenic mesoderm formation regulators.

### BACKGROUND

Heart formation begins during gastrulation with the specification of multipotent cardiovascular progenitors (MCPs) that migrate anteriorly to form the cardiac primordium that assembles into the fully formed heart (Buckingham et al., 2005; Kelly et al., 2014; Meilhac et al., 2015). Intense research over the past two decades has led to the identification of extracellular signals that initiate cardiogenesis (Collop et al., 2006; Foley et al., 2007; Kaltman et al., 2006; Laflamme et al., 2007; Lian et al., 2013; Marvin et al., 2001; Pandur et al., 2002; Schneider and Mercola, 2001; Schultheiss et al., 1997; Yang et al., 2008). In contrast, current knowledge of the intra-cellular mediators controlling this process is very fragmentary. The most commonly used molecules rely on combinations of Wnt inhibitors, Activin, BMP and Wnt agonists, all of which were revealed originally by embryology studies in the 1990s to early 2000s. However, the embryology studies never comprehensively probed the signaling pathways that induce heart but rather took a more hit and miss approach since large-scale approaches were not available at the time.

Discovering such factors by a comprehensive approach would have major implications (1) for appreciating how cardiogenesis is normally initiated, as embryos lacking cardiac progenitors fail to form a heart (Zhao et al., 2008), and (2) for informing the development of regenerative and disease modeling technologies (Mercola et al., 2013; Moretti et al., 2013). Therefore, there is a need to identify cardiogenic mesoderm formation regulators and develop methods of using these regulators for therapeutic and cardiac disease-modeling purposes.

### SUMMARY

This disclosure relates to cardiogenic mesoderm formation regulators.

In one aspect, the disclosure relates to methods of generating a multipotent cardiovascular progenitor cell. The methods involve overexpressing one or more proteins selected from the group consisting of Id1 (Inhibitor of DNA binding 1, HLH protein), Id2 (Inhibitor of DNA Binding 2, HLH Protein), Id3 (Inhibitor of DNA Binding 3, HLH Protein), Id4 (Inhibitor of DNA Binding 4, HLH Protein), Evx1 (Even-Skipped Homeobox 1), and Grrp1 (glycine/arginine rich protein 1) in a stem cell, thereby generating a multipotent cardiovascular progenitor cell.

In some embodiments, the methods involve transfecting the stem cell with a nucleic acid comprising a sequence encoding one or more proteins selected from the group consisting of Id1, Id2, Id3, Id4, Evx1, and Grrp1.

In some embodiments, the protein is Id1, Id2, Id3, or Id4. In some embodiments, the protein is Id1.

In some embodiments, the methods involve overexpressing Mesp1 (Mesoderm posterior protein 1).

In some embodiments, the stem cell is an embryonic stem cell, or an induced pluripotent stem cell. In some embodiments, the stem cell is a human induced pluripotent stem cell, or a mouse induced pluripotent stem cell.

In some embodiments, the nucleic acid is a ribonucleic acid, or a deoxyribonucleic acid.

In one aspect, the disclosure also relates to methods of generating a multipotent cardiovascular progenitor cell. The methods involve delivering into a stem cell a composition comprising one or more proteins selected from the group consisting of Id1, Id2, Id3, Id4, Evx1, and Grrp1.

In some embodiments, the composition includes Id1, Id2, Id3, or Id4. In some embodiments, the composition includes Id1. In some embodiments, the composition further includes Mesp1.

In some embodiments, the composition includes an endosomolytic agent.

In some embodiments, the stem cell is an embryonic stem cell, or an induced pluripotent stem cell. In some embodiments, the stem cell is a human induced pluripotent stem cell, or a mouse induced pluripotent stem cell.

In another aspect, the disclosure relates to methods of generating a multipotent cardiovascular progenitor cell. The methods involve inhibiting the expression or activity of one or both Foxa2 (Forkhead Box A2) and Tcf3 (Transcription Factor 3) in a stem cell, thereby generating a multipotent cardiovascular progenitor cell.

In some embodiments, the methods involve inhibiting Tcf3. In some embodiments, the methods involve inhibiting Foxa2. In some embodiments, the methods involve contacting the stem cell with siTcf3. In some embodiments, the methods involve contacting the stem cell with siFoxa2.

In some embodiments, the stem cell is an embryonic stem cell or an induced pluripotent stem cell. In some embodiments, the stem cell is a human induced pluripotent stem cell or a mouse induced pluripotent stem cell.

In one aspect, the disclosure also relates to a plurality of in vitro multipotent cardiovascular progenitor cells, wherein the multipotent cardiovascular progenitor cells are generated by overexpressing one or more proteins selected from the group consisting of Id1, Id2, Id3, Id4, Evx1, and Grrp1 in a plurality of stem cells.

In some embodiments, the protein is Id1, Id2, Id3, or Id4. In some embodiments, the protein is Id1. In some embodiments, the multipotent cardiovascular progenitor cells are generated by further overexpressing Mesp1.

In some embodiments, the total number of cells is over 10⁶, 10⁷, or 10⁸.

In some embodiments, the stem cells are induced pluripotent stem cells.

The disclosure also relates to a plurality of in vitro multipotent cardiovascular progenitor cells, wherein the multipotent cardiovascular progenitor cells are generated by delivering into a plurality of stem cells a composition comprising one or more proteins selected from the group consisting of Id1, Id2, Id3, Id4, Evx1, and Grrp1.

In some embodiments, the composition includes Id1, Id2, Id3, or Id4. In some embodiments, the composition includes Id1. In some embodiments, the composition further includes Mesp1.

In some embodiments, the total number of cells is over 10⁶, 10⁷, or 10⁸.

In some embodiments, the stem cells are induced pluripotent stem cells.

In another aspect, the disclosure also relates to a plurality of in vitro multipotent cardiovascular progenitor cells, wherein the multipotent cardiovascular progenitor cells are generated by inhibiting the expression or activity of one or both proteins of Foxa2 and Tcf3 in a plurality of stem cells.

In some embodiments, the multipotent cardiovascular progenitor cells are generated by inhibiting Tcf3. In some embodiments, the multipotent cardiovascular progenitor cells are generated by inhibiting Foxa2.

In some embodiments, the total number of cells is over 10⁶, 10⁷, or 10⁸.

In some embodiments, the stem cells are induced pluripotent stem cells.

In another aspect, the disclosure relates to methods of screening for an agent that promotes multipotent cardiovascular progenitor cell formation. The methods involve contacting a cell with a test agent; determining that
(1). the cell has an increased expression or activity of Id1;
(2). the cell has an increased expression or activity of Id2, Id3, or Id4;
(3). the cell has an increased expression or activity of Evx1, Grrp1, or Mesp1;
or
(4). the cell has a decreased expression or activity of Foxa2 or Tcf3; identifying the test agent as an agent that promotes multipotent cardiovascular progenitor cell formation.

In some embodiments, the cell is from an embryonic stem cell line. In some embodiments, the cell is an embryonic stem cell, or an induced pluripotent stem cell. In some embodiments, the cell is a human induced pluripotent stem cell or a mouse induced pluripotent stem cell.

In some embodiments, the agent is a small molecule, a nucleic acid, a peptide, or a protein. In some embodiments, the agent is an oligonucleotide. In some embodiments, the agent is an antisense molecule, a small interfering RNA, or a small hairpin RNA. In some embodiments, the agent is an antibody or an antigen-binding fragment.

In some embodiments, the methods further involve formulating the agent with a pharmaceutically acceptable carrier as a pharmaceutical composition.

In another aspect, the disclosure relates to methods of preparing a pharmaceutical composition. The methods involve formulating an agent that promotes cardiac mesoderm progenitor formation with a pharmaceutically acceptable carrier, wherein the agent has been previously determined to:
(1). increase expression or activity of Id1 in a stem cell;
(2). increase expression or activity of Id2, Id3, or Id4 in a stem cell;
(3). increase expression or activity of Evx1, Grrp1, or Mesp1 in a stem cell;
or
(4). decrease expression or activity of Foxa2 or Tcf3 in a stem cell.

In some embodiments, the stem cell is an embryonic stem cell or an induced pluripotent stem cell.

In some embodiments, the agent has been previously determined to increase expression of Id1 in a stem cell, or has been previously determined to increase expression of Evx1, Grrp1, or Mesp1 in a stem cell.

In some embodiments, the agent is a small molecule, a nucleic acid, a peptide, or a protein.

In some embodiments, the agent is an oligonucleotide. In some embodiments, the agent is an antisense molecule, a small interfering RNA, or a small hairpin RNA. In some embodiments, the agent is an antibody or an antigen-binding fragment.

In another aspect, the disclosure relates to methods of promoting cardiac regeneration in a subject in need thereof. The methods involve generating a plurality of multipotent cardiovascular progenitor cells from a plurality of stem cells; and delivering the plurality of multipotent cardiovascular progenitor cells to the subject.

In some embodiments, the multipotent cardiovascular progenitor cells are generated by overexpressing one or more proteins selected from the group consisting of Id1, Id2, Id3, Id4, Evx1, and Grrp1 in the plurality of stem cells.

In some embodiments, the multipotent cardiovascular progenitor cells are generated by overexpressing Id1 in the plurality of stem cells.

In some embodiments, the multipotent cardiovascular progenitor cells are generated by delivering into the plurality of stem cells a composition comprising one or more proteins selected from the group consisting of Id1, Id2, Id3, Id4, Evx1, and Grrp1.

In some embodiments, the composition includes Id1, Id2, Id3, or Id4. In some embodiments, the composition includes Id1.

In some embodiments, the multipotent cardiovascular progenitor cells are generated by inhibiting the expression or activity of one or both proteins of Foxa2 and Tcf3 in the plurality of stem cells.

In some embodiments, the methods involve inhibiting expression or activity of Tcf3.

In some embodiments, the multipotent cardiovascular progenitor cells are generated by contacting the plurality of stem cells an agent that promotes multipotent cardiovascular progenitor cell formation.

In some embodiments, the stem cells are induced pluripotent stem cells derived from the cells of the subject.

In some embodiments, the subject has myocardial infarction, ischemic heart disease, hypertrophic cardiomyopathy, or congenital cardiomyopathy.

As used herein, the term “effective amount” is an amount sufficient to effect beneficial or desired results (e.g., sufficient to promote multipotent cardiovascular progenitor (MCP) cell formation, or sufficient to provide symptom relief for cardiovascular disorders).

As used herein, the term “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1A. Schematic of screening strategy to identify new regulators of cardiogenic mesoderm differentiation.

FIG. 1B. Immunostaining of Kdr-eGFP (cardiogenic mesoderm) and AlexaFluor568-Foxa2 (endoderm) showing mesoderm and endoderm differentiation in response to siControl at day 6 of differentiation. Scale bar is 50 μm.

FIG. 1C. Immunostaining of Kdr-eGFP (cardiogenic mesoderm) and AlexaFluor568-Foxa2 (endoderm) showing increased mesoderm differentiation in response to siAcvr1b at day 6 of differentiation. Scale bar is 50 μm.

FIG. 1D. Microarray data reveals that 33 transcripts are upregulated (p<0.05) at day 4 in response to siAcvr1b as compared to siControl, 24 hours post-transfection.

FIG. 1E. qRT-PCR confirmation of the microarray results in FIG. 1D, showing that 14 genes are robustly upregulated in response to siAcvr1b as compared to siControl. All qRT-PCR data are normalized to β-actin mRNA levels. Quantitative data are presented as means +/−SD. *p<0.05. All experiments were performed at least in biological quadruplicates.

FIG. 1F. siRNA screen of the 14 candidates from FIG. 1E to evaluate their requirement for cardiogenic mesoderm formation induced by siAcvr1b. Differentiation was quantified by induction of Kdr-eGFP reporter (total integrated intensity, see material and methods for details). siGrrp1, siEvx1 and siId1 strongly repressed siAcvr1b-induced cardiogenic mesoderm.

FIGS. 1G-1J. Representative images of Kdr-eGFP and AlexaFluor568-Foxa2 illustrating results presented in FIG. 1F. Scale bar is 50 μm.

FIG. 1K. qRT-PCR results showing that siGrrp1, siEvx1 and siId1 markedly repress cardiogenic mesoderm-specific marker (Kdr, Mesp1, Snai1, Cdh11) expression. All qRT-PCR data are normalized to β-actin mRNA levels. Quantitative data are presented as means +/−SD. *p<0.05. All experiments were performed at least in biological quadruplicates.

FIGS. 1L-1O. Temporal expression profiles of Id1, Grrp1, Evx1 and Mesp1 in response to siAcvr1b or siControl from day 3 to day 6 of differentiation.

FIGS. 1P-1W. Endogenous expression of Id1, Grrp1, Evx1 and Mesp1 in E7 mouse embryos by in situ hybridization. Whole mount view (FIGS. 1P-1S). Transverse histological section of the proximal region of E7 embryos indicating Id1 (FIG. 1T) expression in the gastrulating epiblast (yellow arrow) and migrating mesoderm (white arrow), Grrp1 (FIG. 1U) expression in the gastrulating epiblast (yellow arrow), as well as Evx1 (FIG. 1V) and Mesp1 (FIG. 1W) expression in the primitive streak (yellow arrow).

FIGS. 1T′-1W′. Endogenous expression of Id1, Grrp1, Evx1 and Mesp1 in E7 mouse embryos by in situ hybridization. Transverse histological section of the proximal region of E7 embryos indicating Id1 (FIG. 1T′) expression (partial view (upper)) in the gastrulating epiblast and migrating mesoderm, Grrp1 (FIG. 1U′) expression (partial view (upper)) in the gastrulating epiblast, as well as Evx1 (FIG. 1V′) (partial view (upper)) and Mesp1 (FIG. 1W′) expression (partial view (upper)) in the primitive streak.

FIG. 1X. Schematic representation of an E7 embryo transverse section illustrating the different domains of expression of the 3 candidates. The gastrulating epiblast (blue) indicates the domain where Id1 and Grrp1 expression overlaps. In the primitive streak region (gray), high levels of Evx1 expression are observed with decreased Grrp1 expression. As cells exit the primitive streak and migrate laterally (purple), cells start to express Mesp1 along with Evx1. As mesoderm cells migrate more anteriorly (orange), cells resume Id1 expression.

FIG. 2A. Schematic of strategy to evaluate the sufficiency (gain of function) of any of three candidates alone or in combination to promote mesoderm differentiation.

FIG. 2B. Kdr-eGFP fluorescence measurement at day 6 of differentiation in mESCs overexpressing all possible combinations of the three candidates plotted relative to uninfected control levels.

FIGS. 2C-2D. Representative images of Kdr-eGFP for Id1 overexpressing vs. control mESCs illustrating the results presented in (B). Scale bar is 50 μm.

FIGS. 2E-2F. Flow cytometry analysis reveals that 61.6% of Id1-overexpressing mESCs differentiate into Kdr-eGFP⁺ mesoderm as compared to 3.65% for control cells at day 6.

FIG. 2G. Alignment and comparison of the mouse (NP_034625.1; SEQ ID NO: 10) Id1 HLH domain to the human (NP_851998.1; SEQ ID NO: 1) Id1 HLH domain using Protein Blast tool reveals the amino acid sequence is 100% identical.

FIG. 2H. qRT-PCR analysis for expression of Id1 in control h9-hESCs vs. h9-hESCs stably overexpressing Id1 measured at day 0 of differentiation.

FIGS. 2I-2J Flow cytometry analysis reveals that 69.8% of Id1-overexpressing h9-hESCs differentiate into KDR⁺ mesoderm at day 5 of differentiation as compared to 9.1% for control h9-hESCs.

FIGS. 2K-2N. Temporal mRNA expression profile of pro-cardiogenic mesoderm genes (Evx1 (FIG. 2K), Grrp1 (FIG. 2L), Mesp1 (FIG. 2M), and Kdr (FIG. 2N)) in mESC lines overexpressing Id1 compared to control mESC lines illustrate that Evx1, Grrp1, and Mesp1 mRNA expression peaks at day 4 of differentiation while Kdr mRNA expression peaks at day 5 of differentiation.

FIGS. 2O-2R. Temporal mRNA expression profiles of EVX1 (FIG. 2O), GRRP1 (FIG. 2P), MESP1 (FIG. 2Q), and KDR (FIG. 2R) in h9-hESCs stably overexpressing Id1 compared to control h9-hESCs.

FIG. 2S. Model summarizing pro-cardiogenic role of Id1 by upregulating the expression of Evx1, Grrp1, and Mesp1 in bi-potent mesendoderm progenitors.

FIG. 3A. Schematic depicting prospective differentiation potential of cryopreserved Id1-induced multipotent cardiovascular progenitors to multiple cardiovascular cell types.

FIG. 3B. mRNA expression profiling for the spontaneous differentiation potential of mESCs stably overexpressing Id1 to cardiac (Myh6, Tnnt2, Actc1), skeletal muscle (Myod1, Myog, Actn3), vascular endothelial (Cdh5, Pecam1) and fibroblasts (Postn, Vim) markers at day 6 and day 15 of differentiation. Quantitative data are presented as means +/−SD. All experiments were performed at least in biological quadruplicates.

FIG. 3C. mRNA expression profiling for the spontaneous differentiation potential of h9-hESCs stably overexpressing Id1 to cardiac (NKX2.5, TNNT2, MYH6, MYL2, MYL7), skeletal muscle (MYOD1, MYOG, ACTN3), vascular endothelial (CDH5, PECAM1), and fibroblasts (POSTN, VIM) markers at day 5 and day 15 of differentiation. Quantitative data are presented as means+/−SD. All experiments were performed at least in biological quadruplicates.

FIG. 3D. Representative immunofluorescence image cardiomyocytes (ACTC1), vascular endothelial cells (CDH5) and fibroblasts (TAGLN) at day 15 of differentiation in h9-hESCs stably overexpressing Id1. Scale bar is 50 μm.

FIG. 3E. Diagram showing quantification of % of ACTC1⁺ (cardiomyocytes), TAGLN⁺ (fibroblasts), CDH5⁺ (vascular endothelial cells) at day 15 of differentiation in h9-hESCs stably overexpressing Id1.

FIG. 3F. Schematic of the workflow for the physiological assessment of cardiomyocytes derived from Id1 overexpressing h9-hESCs using the calcium (Fluo-4) and voltage (VF2.1 Cl) (Miller et al., 2012) sensitive dyes.

FIG. 3G. Representative images illustrating the minimum and maximum changes in fluorescence of Fluo-4 in cardiomyocytes derived from Id1 overexpressing h9-hESCs.

FIG. 3H. Representative calcium transient trace of day 15 cardiomyocytes derived from Id1 overexpressing h9-hESCs.

FIG. 3I. Representative action potential traces of cardiomyocytes derived from Id1 overexpressing h9-hESCs in control conditions.

FIG. 3J. Representative action potential traces of cardiomyocytes derived from Id1 overexpressing h9-hESCs in response to isoproterenol measured optically with VF2.1 Cl.

FIG. 3K. Beat rate quantification of cardiomyocytes derived from Id1 overexpressing h9-hESCs indicating an increase in beating frequency in response to isoproterenol (100 nM) treatment as compared to vehicle and measured with VF2.1 Cl.

FIG. 4A. Schematic predicting that Id1 mediates its pro-cardiogenic effect by targeting and inhibiting repressors of cardiogenic mesoderm differentiation.

FIG. 4B. siRNA-mediated functional screen evaluating the role E proteins (Tcf3, Tcf4, Tcf12) in repressing cardiogenic mesoderm differentiation. Diagram shows fluorescence quantification of Kdr-eGFP in response to all 7 possible siRNA combinations and siControl.

FIGS. 4C-4D. Representative immunofluorescence images of Kdr-eGFP at day 6 of differentiation from mESCs transfected at day 3 with siControl (FIG. 4C) and siTcf3 (FIG. 4D). Scale bar is 50 μm.

FIG. 4E. qRT-PCR validation showing that 17 genes are downregulated at day 4 in response to siAcvr1b as compared to siControl, 24 hours post transfection.

FIGS. 4F-4G. siRNA-mediated functional screen evaluating if downstream targets of Acvr1b signaling are involved in the repression of cardiogenic mesoderm differentiation. Diagram shows fluorescence quantification of Kdr-eGFP where only a siRNA directed against siFoxa2 is able to promote cardiogenic mesoderm differentiation (FIG. 4F). Representative Kdr-eGFP immunofluorescence images of siFoxa2 (FIG. 4G). Scale bar is 50 μm.

FIG. 4H. qRT-PCR shows that Foxa2 expression is downregulated in Id1-overexpressing mESCs as compared to control.

FIGS. 4I-4K. qRT-PCR for cardiogenic mesoderm markers (Mesp1, Snai1, Cdh11 and Kdr) shows that the co-transfection of siFoxa2 and siTcf3 further enhances cardiogenic mesoderm differentiation as compared to siTcf3 or siFoxa2 alone (FIG. 4I). Diagram showing fluorescence quantification of Kdr-eGFP (FIG. 4J) and representative image (FIG. 4K) of siTcf3+siFoxa2 condition. Scale bar is 50 μm.

FIG. 4L. Model showing Id1 repressive role on Tcf3 and Foxa2 activity to promote cardiogenic mesoderm differentiation.

FIG. 5A. Xid2 mRNA was injected equatorially into two blastomeres on one side of four-cell stage embryos.

FIG. 5B. Mouse HLH domain of Id1 (NP_034625.1) was aligned and compared to all Xenopus laevis HLH (yellow) domains of id proteins using Protein Blast tool (blast.ncbi.nlm.nih.gov). With 79% of identical amino acids, Xid2, (NP_001081902.1) (A), is the closest ortholog to Id1.

FIGS. 5C-5F. Unilaterally injected embryos (as in FIG. 5A) cultured to gastrula stage (stage 10.5) in whole mount (FIGS. 5C and 5E) or transversely bisected (FIGS. 5D and 5F) and probed for mesoderm marker Xbra (FIGS. 5C and 5D) and cardiogenic mesoderm Xmespb (FIGS. 5E and 5F) expression. Yellow arrows indicate expansions of both the Xbra and Xmespb domains in the Xid1 injected side.

FIGS. 5G-5I. Unilaterally injected embryos cultured to early tailbud stage (stage 25) in whole mount and probed for Xnkx2.5 expression. Yellow arrows indicated an expansion of the Xnkx2.5 domain in the Xid2 injected side of the embryo.

FIG. 6A. Schematic illustrating the generation and analysis of Id1-4 mutant embryos using CRISPR/Cas9 technology. Two sgRNAs per gene (targeting the translational start site and the HLH domain) were injected into single cell mouse zygotes alongside Cas9 mRNA. Zygotes were reimplanted and harvested at stages

E7.5-E8.5. Resulting embryos were genotyped by DNA deep sequencing and cardiac gene expression was assessed via whole mount in situ hybridization.

FIGS. 6B-I and 6K-V. In situ hybridization results from the most severe Id1-4 mutants, compared to wild type (individual mutants are marked by a #), plus one lesser affected mutant (FIG. 6P); analyzing Smarcd3 at E7.75 (FIGS. 6B-6E), Tbx5 at E8.0 (FIGS. 6F-6I), Nkx2.5 at E8.25 (FIGS. 6K-6N; plus transverse sections through the heat tube forming region, FIG. 6L′, FIG. 6N′), Nkx2.5 at E8.5 (FIGS. 6O-6R), and Tbx5 at E8.5 (FIGS. 6S-6V). Yellow arrowheads point to the missing heart tube (or missing heart tube forming region at cardiac crescent stages) in Id1-4 mutants; white arrowhead indicates a malformed heart tube; black arrows indicate the plane of transverse sectioning through the heart tube forming region; black dashed arrows point to posterior-lateral cardiac regions. See Supplemental methods for detailed sequencing results of mutant embryos.

FIG. 7. Id1 orchestrates cardiogenic mesoderm differentiation in vertebrates. Id1 controls the activation of cardiogenic mesoderm differentiation program in mesendoderm progenitors by inhibiting the activity of repressors (Tcf3, Foxa2) while promoting the expression of activators of cardiogenic mesoderm differentiation (Evx1, Grrp1, Mesp1). The Id1-controlled network consistently induces robust cardiogenic mesoderm (Mesp1, Kdr) differentiation from pluripotent cells. Resulting multipotent cardiovascular progenitors spontaneously differentiate into contracting cardiomyocytes, vascular endothelial cells and fibroblasts both in mouse and human ESCs.

FIG. 8A. RT-qPCR analysis shows the establishment of mESC cell-line overexpressing all possible combinations of Id1, Evx1 and Grrp1 as compared to control mESCs. Quantitative data are presented as means+/−SD. All experiments were performed at least in biological quadruplicates.

FIGS. 8B-8G. Representative images of Kdr-eGFP fluorescence at day 6 of differentiation in mESCs overexpressing all possible combinations of the three candidate genes. Scale bar is 50 μm.

FIG. 9A. Representative immunofluorescence images for cardiomyocytes (Actc1) at day 15 of differentiation. Scale bar is 50 μm.

FIG. 9B. Representative immunofluorescence images for vascular endothelial cells (Pecam1) at day 15 of differentiation. Scale bar is 50 μm.

FIG. 10A. qRT-PCR analysis showing siTcf3-mediated knock-down efficiency as compared to siControl at day 5 of differentiation.

FIG. 10B. qRT-PCR analysis showing siFoxa2-mediated knock-down efficiency as compared to siControl at day 5 of differentiation.

FIG. 11A. Dorsal view of the embryos. Embryos were hemilaterally injected at 4-cell stage as in FIG. 5 and cultured to early tailbud stage (stage 25). Whole mount in situ hybridization probes for skeletal muscle marker Xmlc expression.

FIG. 11B. Control side of the embryos.

FIG. 11C. Injected side of the embryos. Yellow arrows indicate decreased and disorganized pattern of expression in the injected side as compared to control side.

FIG. 12. Summary of genotype information of mouse embryos analyzed in loss of function study. DNA sequences of 24 mutant embryos were analyzed, and variant alleles were recorded using IGV genome browser (Broad Institute). Blue box marks wild type allele, red box marks null alleles, orange box marks large (>50 bp) in-frame deletion, yellow box marks small (<25bp) in-frame deletion. Phenotypes are annotated as follows: N: no cardiac defect, S: severe loss (or absence) of medial staining in cardiac crescent, A: absence of heart tube, CB: cardiac bifida, LF: looping failure. Two embryo stages are reported: CC: cardiac crescent stage and HT: heart tube stage. Four cardiac markers were tested: Smarcd3, Tbx5, Nkx2. 5, and Fgf8.

FIG. 13A. Ventral view of embryo #9 after Tbx5 in situ hybridization.

FIG. 13B. Transverse section at heart tube level confirms the absence of anatomical heart tube and mesoderm between neural tube and foregut.

FIG. 13C. More posterior transverse section showing expression of Tbx5 expression in cardiac splanchnic mesoderm marked by yellow arrows.

FIG. 14 shows the sequences for several cardiogenic mesoderm formation regulators.

### DETAILED DESCRIPTION

This disclosure relates to cardiogenic mesoderm formation regulators. Basic helix-loop-helix (bHLH) transcription factors Mesp1 and Mesp2 (Saga et al., 2000) under the control of T-box factor Eomes (Costello et al., 2011), regulate at least part of this process in mesoderm cells by directing the expression of genes involved in cardiac specification (Hand2, Gata4, Nkx2.5, Myocd) and cellular migration (Prickle1 and RasGRP3), while actively repressing genes regulating pluripotency (Oct4, Nanog, Sox2), early mesoderm (T), and endoderm (Foxa2, Sox17) fates (Bondue et al., 2008; Chiapparo et al., 2016; Costello et al., 2011). Although these observations suggest that Mesp1/2 genes could act as master regulators of multipotent cardiovascular specification, retrospective lineage analysis (Saga et al., 2000; Yoshida et al., 2008) and in vitro differentiation studies (Chan et al., 2013) have shown that Mesp1-expressing cells also contribute to a wide range of non-cardiac derivatives, including hematopoietic precursors, skeletal muscle cells, and head mesenchyme. Therefore, additional effectors responsible for specifying cardiac cell fate remain to be discovered.

Attenuating Acvr1b signaling in mesendoderm segregates cardiogenic mesoderm from endoderm, whereas persistent Acvr1b signaling drives cells to form endoderm (Colas et al., 2012). Thus, it is hypothesized that genes induced in response to Acvr1b signaling inhibition might be key determinants of cardiogenic mesoderm formation. This disclosure took a systematic approach to functionally test the necessity and sufficiency of the genes modulated by Acvr1b signaling blockade. Unexpectedly, Id1, a helix-loop-helix transcriptional regulator, was identified as a single factor sufficient to control the emergence of Kdr⁺ multipotent cardiovascular progenitors both in mouse and human embryonic stem cells. Mechanistically, Id proteins mediate their evolutionarily conserved role by activating the expression of agonists of cardiogenic mesoderm formation (Evx1, Grrp1 and Mesp1), while inhibiting antagonists' activity (Tcf3, Foxa2). Finally, CRISPR/Cas9-mediated deletion of all four Id family members in mouse blocked early cardiac progenitor formation, and yielded embryos without a heart. The heartless phenotype was unique to the quadruple knockout, indicating compensatory or redundant functions of the Id proteins in formation of cardiac mesoderm. These findings reveal an unexpected role for Id proteins as the earliest determinants of cardiac cell fate in vertebrates.

A number of studies showed that cardiac progenitor cells made from embryonic stem cells (ESCs) form new cardiomyocytes and improve cardiac function in rodent (Christoforou, N., et al. PLoS One 5, e11536 (2010); Tomescot, A., et al. Stem Cells 25, 2200-2205 (2007)), non-human primate (Blin, G., et al. J Clin Invest 120, 1125-1139 (2010)) and sheep MI models (Menard, C., et al. Lancet 366, 1005-1012 (2005)). These studies showed that the progenitor cells improved cardiac function and were safe. Despite these promising results, there has not been progress in developing stem cell-derived cardiac progenitors for human use, largely because there has not been a reproducible and robust means to produce such cells. Illustrating this point, a reference involving a small clinical trial using ESC-derived cardiac progenitor cells indicates that the challenges in producing enriched progenitors are, in part, that the cells used in the human clinical trial might not be as cardiac-committed as in the preceding animal studies (see Menasché et al., Eur Heart J (2015) 36: 743-750). This disclosure presents methods to overcome the roadblock to producing enriched populations of cardiac committed progenitors.

This disclosure demonstrates that simple overexpression of Id1 in hESCs (huma embryonic stem cell) or hiPSCs (human induced pluripotent stem cell) is sufficient to generate large amounts (>10⁸ cells/batch) of cryopreservable and bona fide multipotent cardiovascular progenitors with remarkable abilities to spontaneously differentiate into beating cardiomyocytes (˜70% efficiency). These combined properties enable at least two major applications for Id1-programmed progenitors: (1) as a promising transplantable cell population to test for cardiac regenerative purposes after myocardial injury, and (2) as a novel source of cells enabling large-scale production of hESC or hiP SC-derived cardiomyocytes suitable for in vitro studies of cardiomyocyte physiology.

### Cardiogenic Mesoderm Formation Regulators

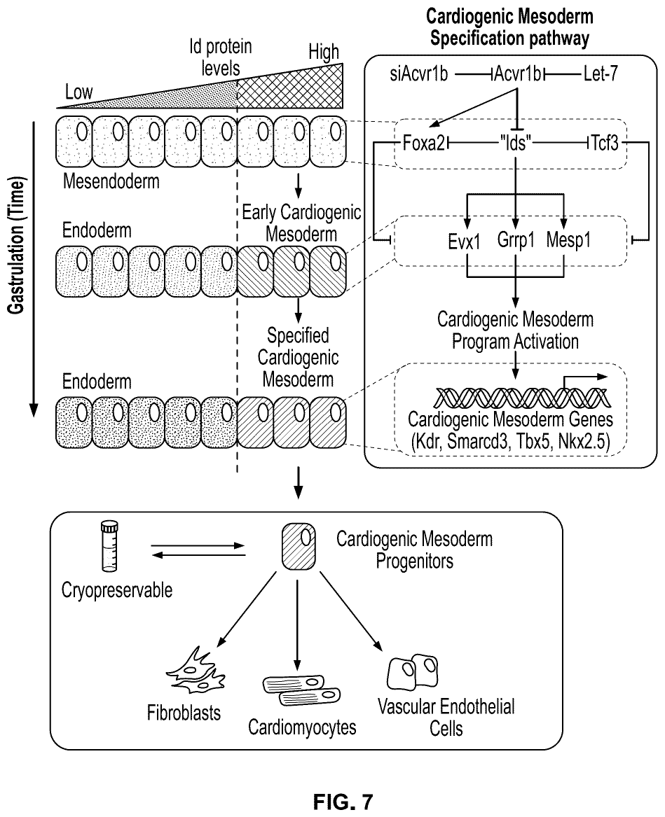

Unraveling the molecular mechanisms controlling cardiogenic mesoderm specification is crucial to understand how heart formation is normally initiated during embryonic development. This disclosure reveals that cardiogenic mesoderm specification is tightly regulated in bi-potent mesendoderm progenitors by an antagonistic interplay between Id proteins (Id1, Id2, Id3, Id4) and the Acvr1b (Activin A Receptor Type 1B) signaling pathway. Stereotypically, high Acvr1b signaling activity represses Id genes expression and biases mesendoderm progenitors to differentiate towards endoderm. Conversely, attenuation of Acvr1b signaling in these cells de-represses Id gene transcription that, in turn, promotes cardiogenic mesoderm specification (FIG. 7). A central finding in this disclosure is the ability of Id proteins to override pro-endoderm cues, induced by high Acvr1b signaling, and promote cardiogenic mesoderm differentiation instead. The functional dominance of Id proteins over Acvr1b signaling implies that molecules controlling the spatial and quantitative distribution of Id proteins are likely to be crucial regulators of cardiogenic mesoderm formation. FIG. 7 summarizes the function of each cardiogenic mesoderm formation regulator in multipotent cardiovascular progenitor cell formation. As shown in FIG. 7, Id1, Id2, Id3, Id4, Evx1, Grrp1, and Mesp1 are promotors of multipotent cardiovascular progenitor cell formation. In contrast, Foxa2 and Tcf3 inhibit multipotent cardiovascular progenitor cell formation. Therefore, inhibiting the expression or the activity of Foxa2 and Tcf3 can promote cardiogenic mesoderm differentiation.

Id1 (Inhibitor of DNA binding 1, HLH protein), Id2 (Inhibitor of DNA binding 2, HLH protein), Id3 (Inhibitor of DNA binding 3, HLH protein) and Id4 (Inhibitor of DNA binding 4, HLH protein) belong to the inhibitor of DNA binding (Id) family. Members of Id family are transcriptional regulators, and contain a helix-loop-helix (HLH) domain. Id proteins can inhibit the functions of basic helix-loop-helix transcription factors in a dominant-negative manner by suppressing their heterodimerization partners through the HLH domains.

Id1 is encoded by Id1 gene (NM_181353.2, SEQ ID NO: 11; NM_002165.3, SEQ ID NO: 20). The amino sequence (NP_851998.1) of Id1 is set forth in SEQ ID NO: 1. Id1 has no DNA binding activity and can inhibit the DNA binding and transcriptional activation ability of basic HLH proteins with which it interacts (Benezra R, Davis R L, Lockshon D, Turner D L, Weintraub H (1990). “The protein Id: a negative regulator of helix-loop-helix DNA binding proteins”. Cell. 61 (1): 49-59). Id2 is encoded by Id2 gene (NM_002166.4; SEQ ID NO: 12). The amino sequence (Q02363-1) of Id2 is set forth in SEQ ID NO: 2. Id3 is encoded by Id3 gene (NM_002167.4; SEQ ID NO: 13). The amino sequence (Q02535-1) of Id3 is set forth in SEQ ID NO: 3. Id4 is encoded by Id4 gene (NM_001546.3; SEQ ID NO: 14). The amino sequence (P47928-1) of Id4 is set forth in SEQ ID NO: 4.

Evx1 (Even-Skipped Homeobox) is a homeobox transcription factor (NM_001989; SEQ ID NO: 15). It is a member of the even-skipped homeobox family characterized by the presence of a homeodomain closely related to the Drosophila even-skipped (eve) segmentation gene of the pair-rule class. Evx1 plays an important role as a transcriptional repressor during embryogenesis. The amino acid sequence of Evx1 (P49640-1) is set forth in SEQ ID NO: 5.

Grrp1 (glycine/arginine rich protein 1; NM_024869; SEQ ID NO: 16), also known as FAM110D (Family with Sequence Similarity 110 Member D), is a paralog of FAM110A. The amino acid sequence of Grrp1 (Q8TAY7-1) is set forth in SEQ ID NO: 6.

Mesp1 (Mesoderm posterior protein 1; NM_018670.3; SEQ ID NO: 17) plays an important role in the epithelialization of somitic mesoderm and in the development of cardiac mesoderm. The amino acid sequence of Mesp1 (Q9BRJ9-1) is set forth in SEQ ID NO: 7.

Foxa2 (Forkhead Box A2; NM_021784.4, SEQ ID NO: 18; NM_153675.2, SEQ ID NO: 21) is known to be involved in embryonic development, and is involved in the development of multiple endoderm-derived organ systems such as the liver, pancreas and lungs. The amino acid sequence of Foxa2 (Q9Y261-1) is set forth in SEQ ID NO: 8.

Tcf3 (Transcription Factor 3; NM_001136139.3, SEQ ID NO: 19; NM_001351778.1, SEQ ID NO: 22; NM_001351779.1, SEQ ID NO: 23; NM_003200.4, SEQ ID NO: 24), also known as E2A; E47. The amino acid sequence of Tcf3 (P15923-1) is set forth in SEQ ID NO: 9.

### Positioning Id Genes in the Context of Mesp1 Pro-Cardiogenic Activity

Many transcription factors have been shown to be essential for cardiac development. Among them, Mesp1 is expressed the earliest and is sufficient to directly promote cardiac specification in mesoderm progenitors. Importantly, the gain of function experiments show that Id1/Xid2 is sufficient to direct Mesp1/Xmespb expression in both mouse and human ESCs as well as in Xenopus embryos, and subsequently promote cardiogenic mesoderm differentiation. These observations suggest that Id proteins exert at least part of their pro-cardiogenic effect through the upregulation of Mesp genes. Since Id proteins do not directly bind DNA to promote gene transcription, the Id-mediated upregulation of Mesp genes is likely to be indirect and may result from the inhibition of repressors of Mesp gene transcription. Indeed, the data in this disclosure shows that siRNA-mediated knock-down of canonical Id protein target Tcf3, and Id downstream target Foxa2, are sufficient to independently upregulate Mesp1 expression and promote cardiogenic mesoderm differentiation. Consistent with the model, previous studies have shown that Tcf3 agonistically interacts with Smad2/3 to upregulate Nodal (Activin) target genes in mesendoderm progenitors (Yoon et al 2011), while Foxa2 is the earliest known determinant of definitive endoderm (Stainier, 2002; Viotti et al., 2014) and is sufficient to induce hepatocyte-like cell differentiation in ESCs and adult fibroblasts (Huang et al., 2011; Sekiya and Suzuki, 2011). These functions suggest that the dual blockade of Tcf3 and Foxa2 in mesendoderm progenitors drives concomitant two processes: (1) the activation of cardiac specification via Mesp upregulation, and (2) the prevention of endoderm specification.

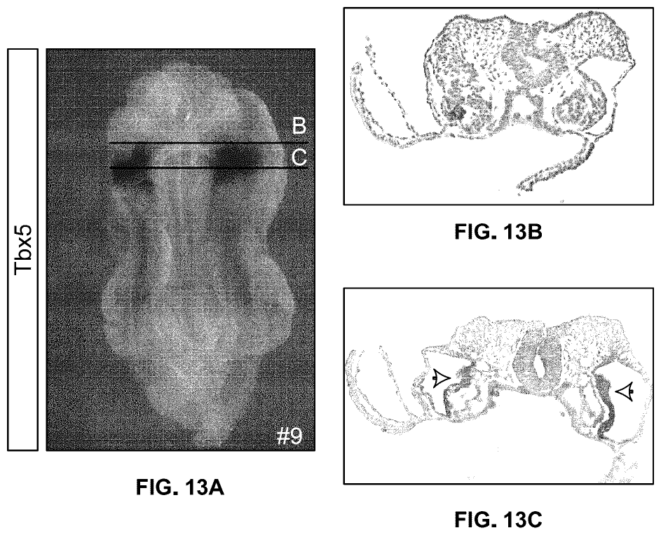

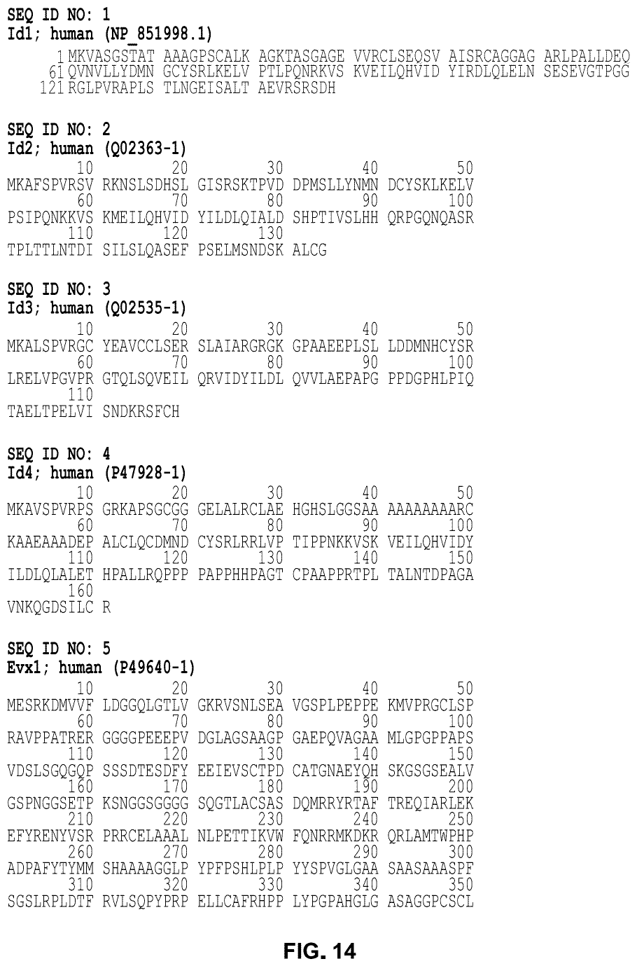

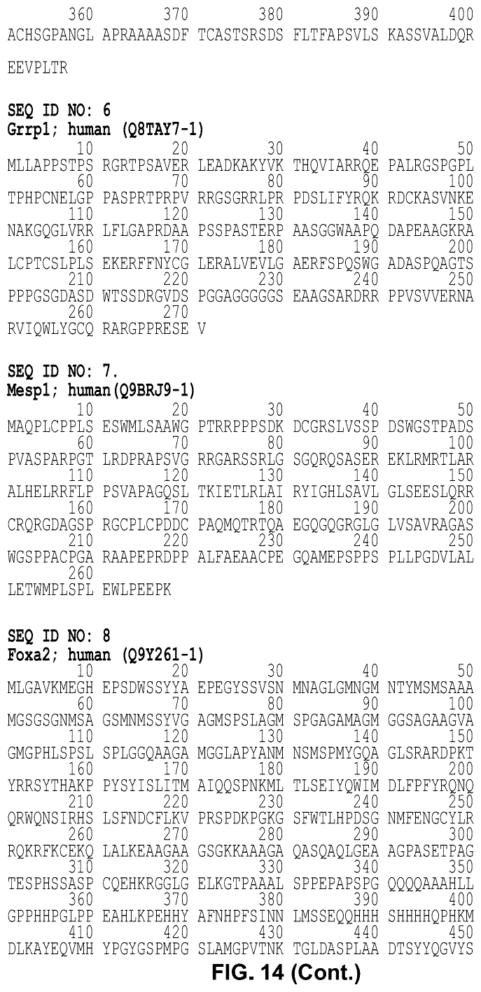

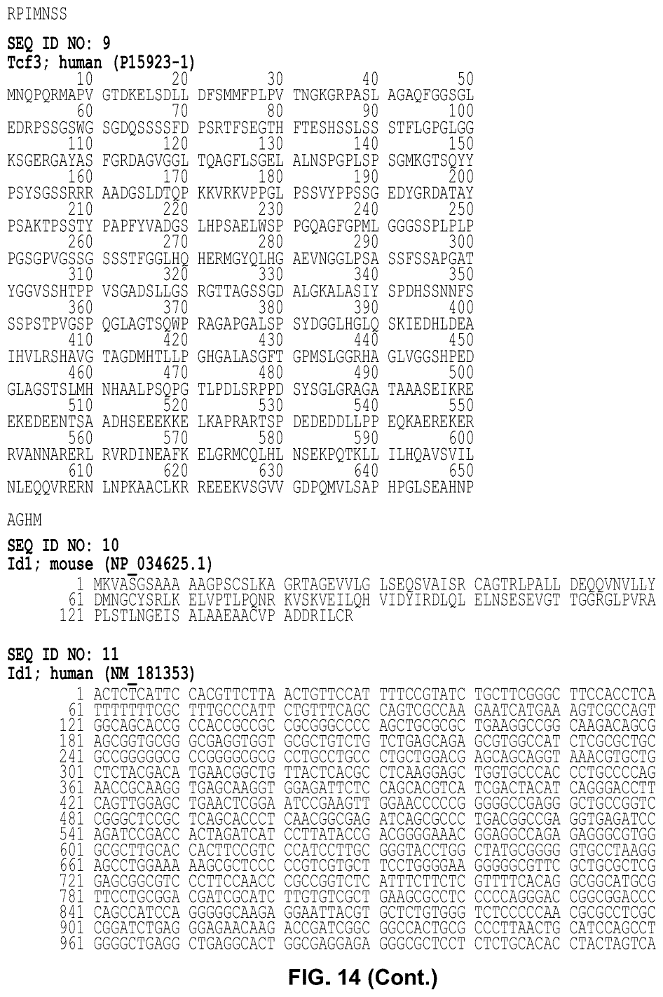

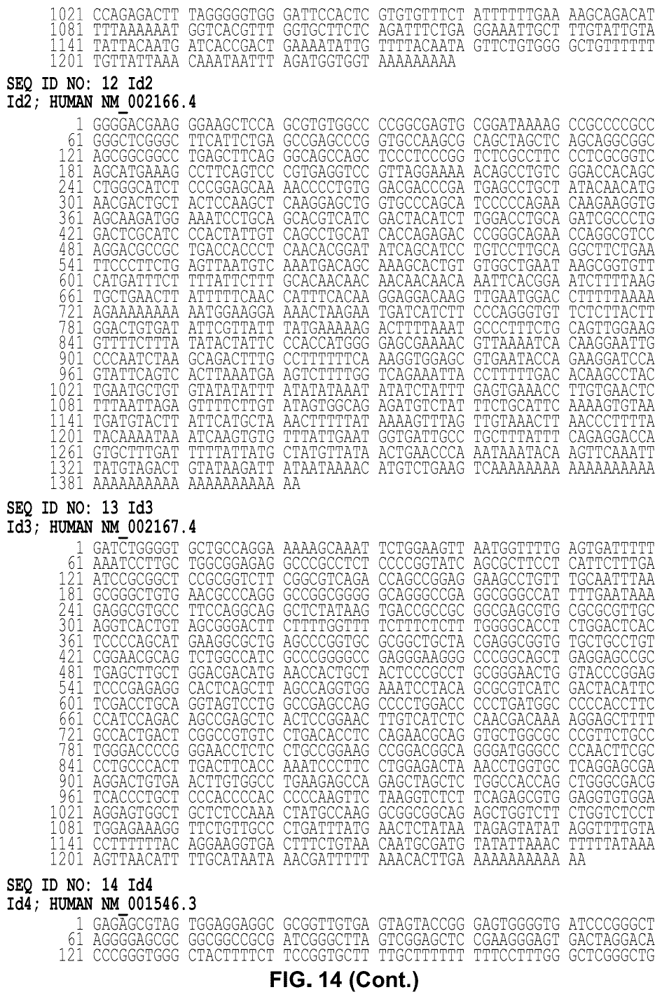

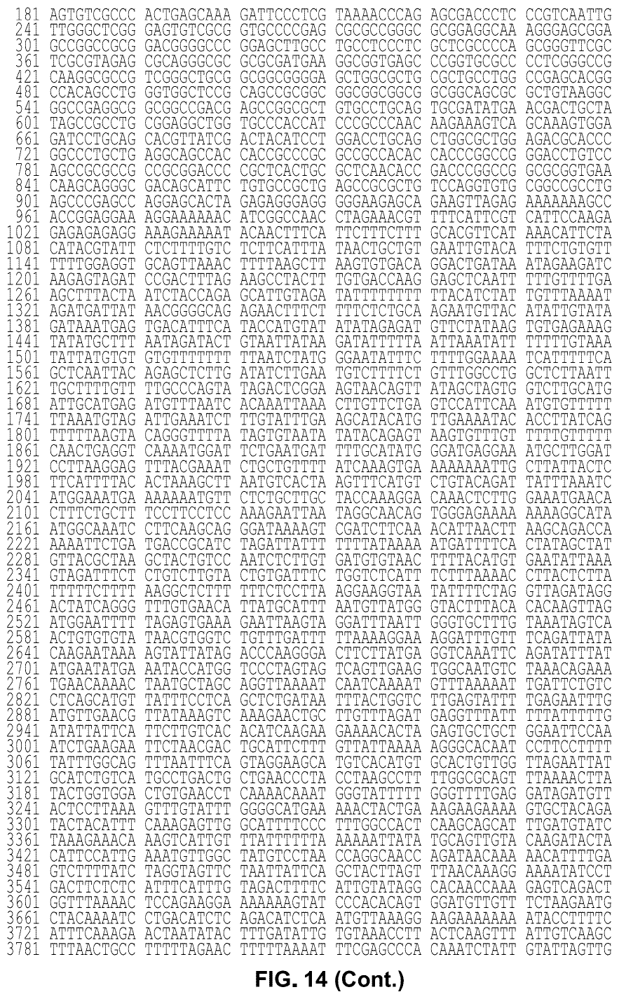

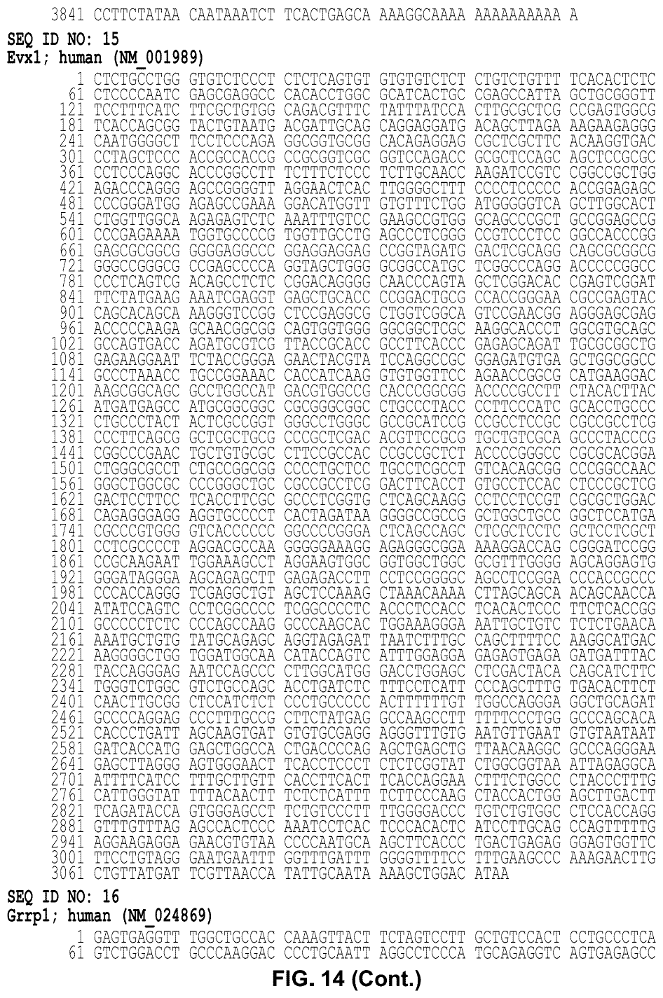

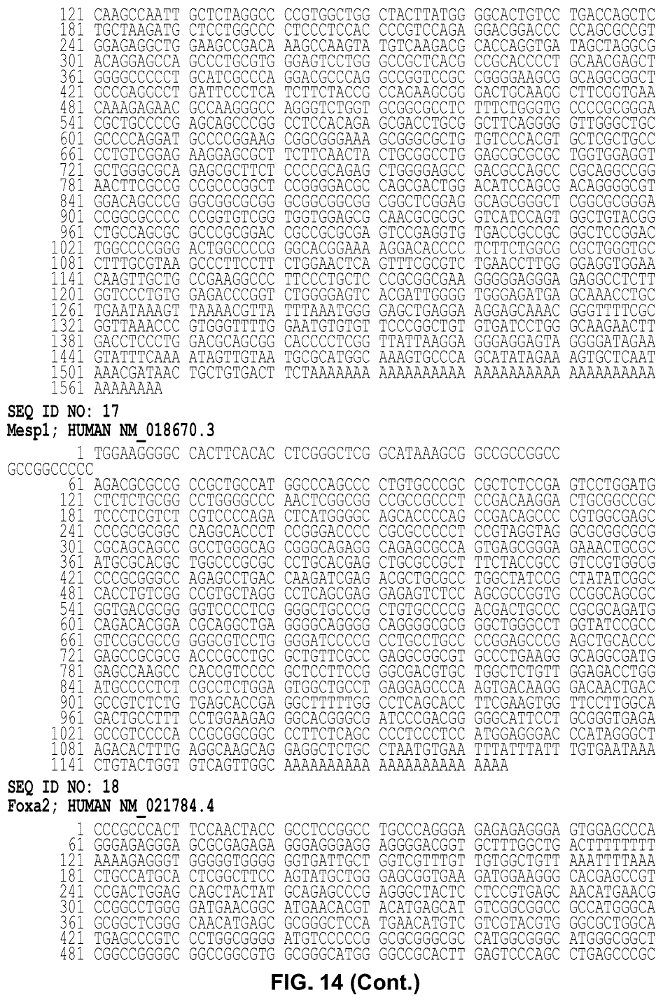

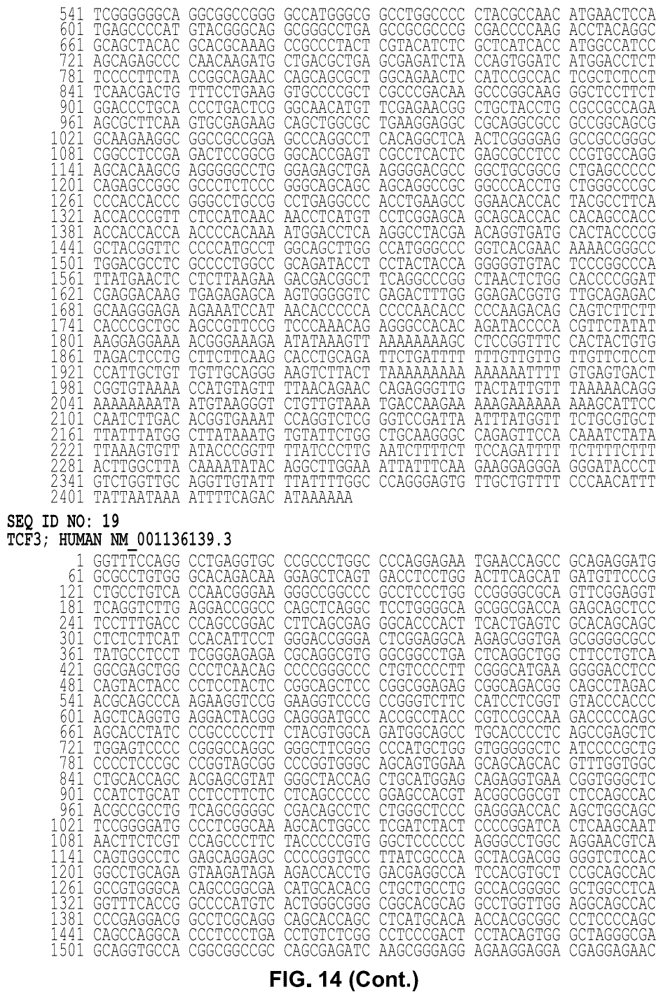

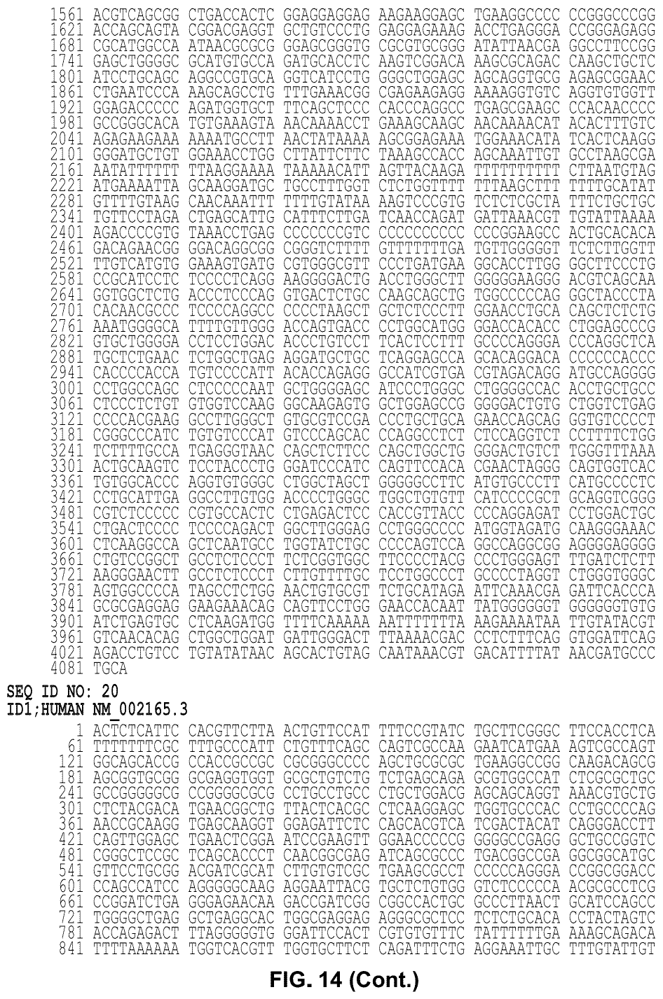

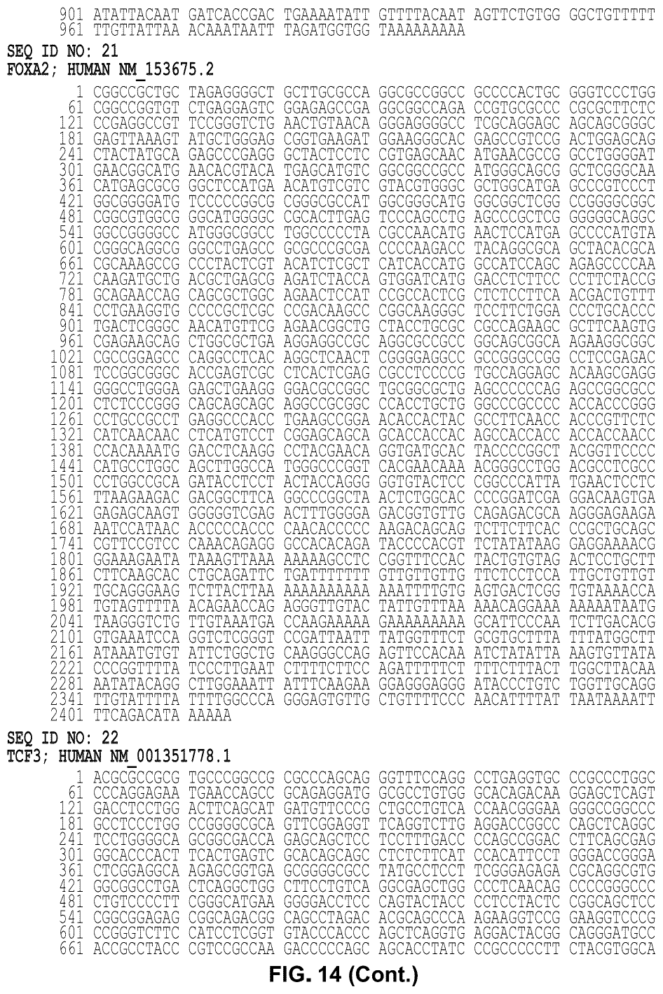

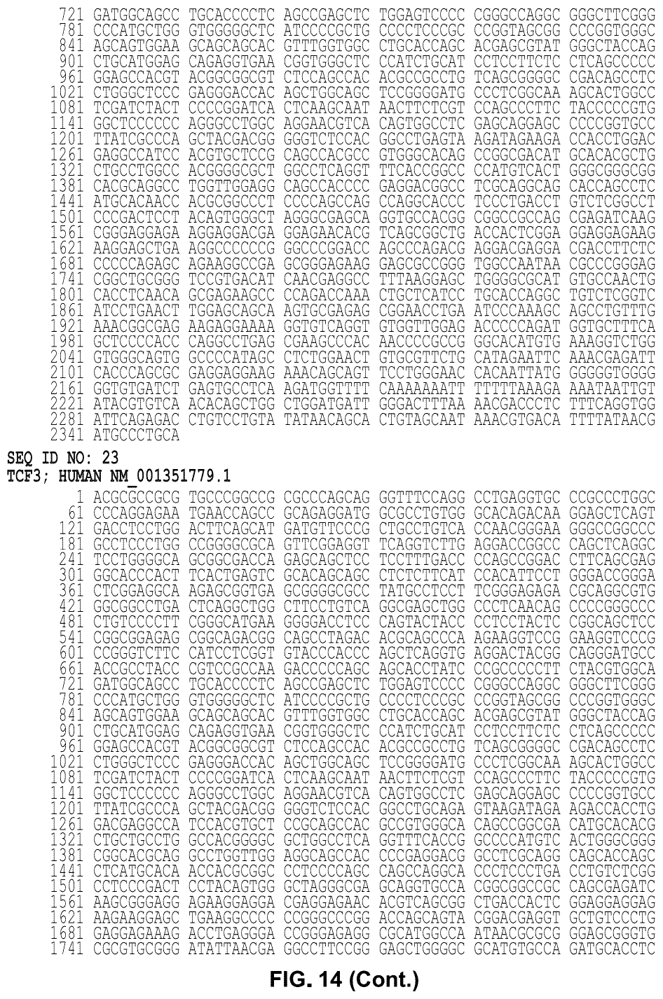

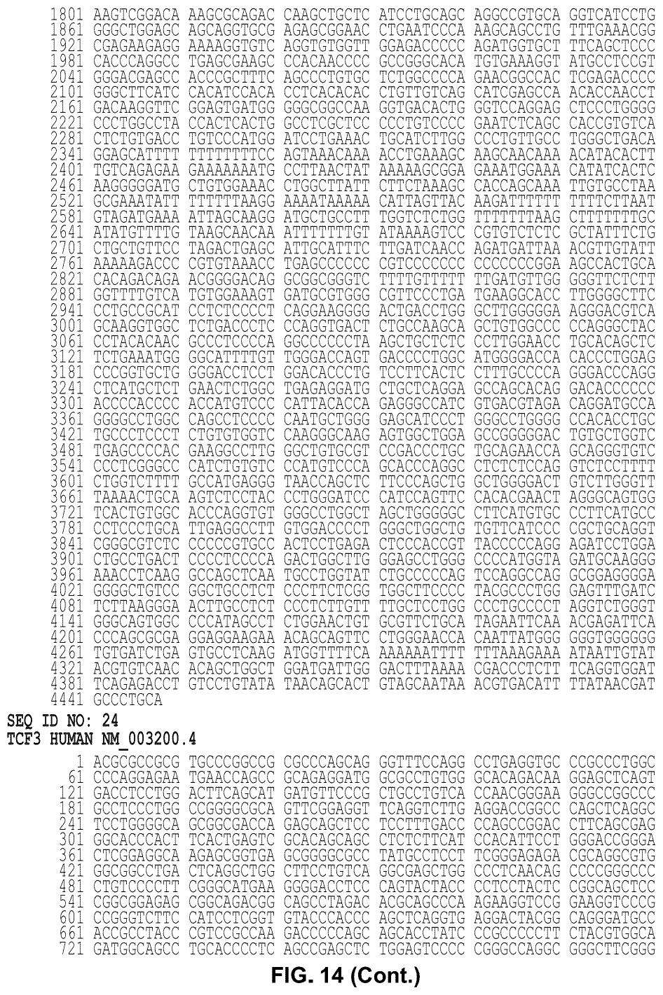

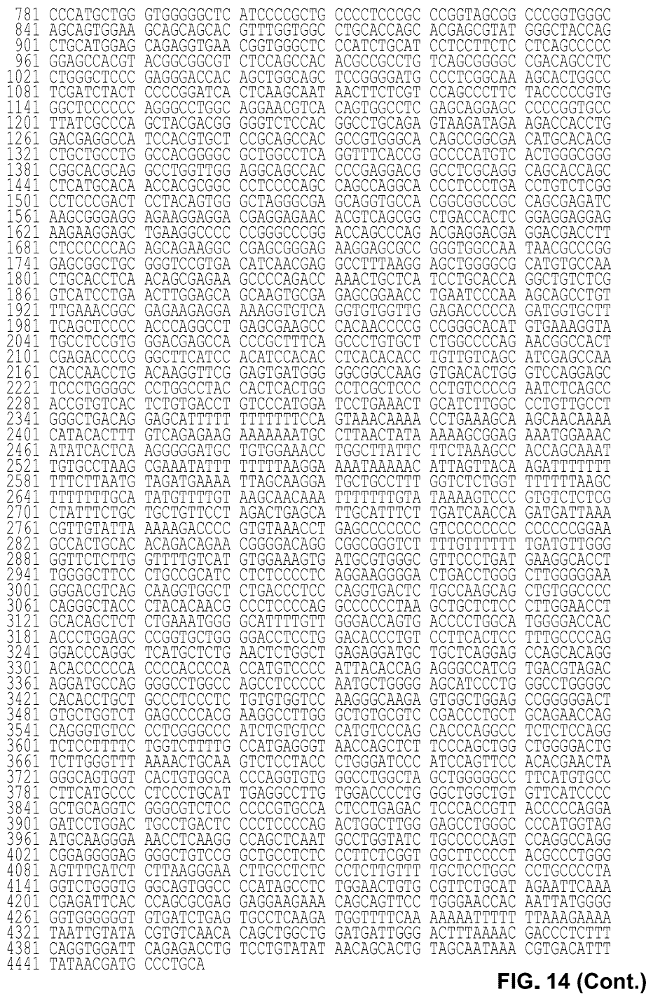

It is well described that most of the heart myocardium in mammals derives from two distinct populations of cardiac progenitors, referred to as heart fields (Cai et al., 2003; Kelly et al., 2001; Meilhac et al., 2004; Meilhac et al., 2015). It is, however, not known whether similar or distinct molecular mechanisms regulate cardiac specification in these two cell populations. The loss of function results show that embryos lacking functional Id1-4 genes fail to express cardiogenic mesoderm markers (Smarcd3, Tbx5 and Nkx2.5) in the most anterior region of the cardiac crescent at E7.75, and subsequently develop without forming a heart tube. In contrast, posterior expression of these genes in the cardiac crescent is maintained and histological sectioning confirmed the presence of splanchnic cardiac mesoderm, posterior to the presumptive heart tube location (FIGS. 13A-13C). Collectively, these observations suggest that only the most anterior subset of cardiac progenitors require Id1-4 activity for their specification. Thus, Id genes normally specify first heart field progenitors that form the early heart tube. These findings also imply that cardiogenic mesoderm specification is not a singular process and can be initiated in an Id-dependent (first heart field progenitors) or Id-independent (posterior cardiac progenitors) manner during embryonic development.

Furthermore, BMP (Bone morphogenetic protein) signaling directly activates Id1 transcription (Hollnagel et al., 1999; Katagiri et al., 2002; Korchynskyi and ten Dijke, 2002; Lopez-Rovira et al., 2002). Conversely, the finding that Acvr1b signaling represses Id1/3 gene expression is consistent with the ability of a small molecule inhibitor of the Nodal receptor (SB431542) to upregulate Id1 transcripts in mESCs (Galvin et al., 2010), and reinforces the role of Acvr1b signaling in opposing cardiac cell fate acquisition during gastrulation. In summary, high Id protein levels in mesendoderm progenitors constitute a dominant molecular cue that is sufficient to trigger and orchestrate cardiogenic mesoderm specification in vertebrates.

### Methods of Generating Multipotent Cardiovascular Progenitor Cells (MCP)

Mesoderm is one of the three primary germ layers in the very early embryo. It forms mesenchyme, mesothelium, non-epithelial blood cells and coelomocytes. Multipotent cardiovascular progenitor cells (or known as cardiogenic mesoderm progenitor cells) are differentiated from mesoderm cells. Multipotent cardiovascular progenitor cells resemble cells in the developing embryo that can develop into various cells of the heart, including cardiomyocytes, vascular endothelial cells, vascular smooth muscle cells and cardiac fibroblasts. Therefore, multipotent cardiovascular progenitor cells are in principle useful for regenerative medicine and disease-modeling.

The present disclosure provides methods of generating multipotent cardiovascular progenitor cells. In one aspect, the methods involve overexpressing one or more proteins (or protein variants thereof) selected from the group consisting of Id1, Id2, Id3, Id4, Evx1, Mesp1, and Grrp1 in a stem cell, thereby generating a multipotent cardiovascular progenitor cell. Overexpression can be achieved by various methods known in the art, e.g., by introducing nucleic acids into cells. Methods for introducing nucleic acids into cells include, but are not limited to, virus infection, transfection, electroporation, lipofection, and may other methods known in the art.

Viral vectors are often used to deliver genetic material into cells. This process can be performed inside a living organism (in vivo) or in cell culture (in vitro). Commonly used virus vectors include retrovirus, lentivirus (e.g., lentivectors such as pCDH-CMV), adenovirus, and adeno-associated virus, etc.

Overexpression can also be achieved by transfecting the cell with a nucleic acid (e.g., a ribonucleic acid, a deoxyribonucleic acid, a modified RNA, or a modified DNA). The nucleic acid can encode one or more proteins selected from the group consisting of Id1, Id2, Id3, Id4, Evx1, Mesp1, and Grrp1, or encode the protein variants of these cardiogenic mesoderm formation regulators. In addition, in some embodiments, inhibitory nucleic acids are used. For example, multipotent cardiovascular progenitor cells can be generated by contacting cells with Foxa2 and/or Tcf3 inhibitory nucleic acids. In some embodiments, the nucleic acid is a modified RNA.

In some embodiments, overexpression can be achieved by delivering an agent to a cell, wherein the agent stimulates the expression of endogenous Id1, Id2, Id3, Id4, Evx1, Mesp1, and/or Grrp1 (i.e. a compound that has the same effect as Id1, Id2, Id3, Id4, Evx1, Mesp1, and/or Grrp1 when administered to a subject).

Multipotent cardiovascular progenitor cells can also be generated by delivering a composition comprising one or more proteins (or protein variants thereof) selected from the group consisting of Id1, Id2, Id3, Id4, Evx1, Mesp1, and Grrp1 into a stem cell.

As used herein, a protein variant is a peptide that has a sequence that is at least 70%, 80%, 90%, 95%, 96%, 97%, 98%, 99% identical to the proteins described in this disclosure (e.g., Id1, Id2, Id3, Id4, Evx1, Mesp1, Grrp1, Foxa2, and Tcf3). To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90%, 95%, or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. For purposes of the present invention, the comparison of sequences and determination of percent identity between two sequences can be accomplished using a Blossum 62 scoring matrix with a gap penalty of 12, a gap extend penalty of 4, and a frameshift gap penalty of 5. Therefore, multipotent cardiovascular progenitor cells can be generated by delivering a composition comprising the protein variants of Id1, Id2, Id3, Id4, Evx1, Mesp1, and/or Grrp1 into a stem cell, or overexpression the protein variants of Id1, Id2, Id3, Id4, Evx1, Mesp1, and/or Grrp1 in a stem cell. For example, multipotent cardiovascular progenitor cells can be generated by transfecting the cell with a nucleic acid encoding an Id1 protein variant. The Id1 protein variant can be at least 70%, 80%, 90%, 95%, 96%, 97%, 98%, 99% identical to SEQ ID NO: 1.

The composition that contains one or more proteins selected from the group consisting of Id1, Id2, Id3, Id4, Evx1, Mesp1, and Grrp1, or contains protein variants of Id1, Id2, Id3, Id4, Evx1, Mesp1, and/or Grrp1 can further include an endosomolytic agent. Macromolecular delivery typically involves the endocytic pathway as a route of cellular entry. However, endosomal entrapment severely limits the efficiency with which macromolecules penetrate the cytosolic space of cells. Endosomolytic agents can facility the escape of macromolecular from endosomes. Some exemplary endosomolytic agents include one domain of the HIV transactivating transcriptional activator, or trans-activator of transcription (“the TAT domain”). By mediating endosomal leakage, these endosomolytic agents can deliver proteins into cells after a simple co-incubation procedure. Delivery does not require a binding interaction between TAT and a macromolecular. Multiple molecules can be delivered simultaneously. These methods of using the TAT endosomolytic agents are described, e.g., in US Pub. No. 2015/0099690, which is incorporated by reference in its entirety. Some other endosomolytic agents and cell delivery systems are described, e.g., in U.S. Pat. No. 6,849,272, B1, which is incorporated by reference herein in its entirety.

In some embodiments, the nucleic acid, the proteins, or any other agents or compositions as described in the present application are delivered through exosomal delivery. Exosomes are small intracellular membrane-based vesicles with different compositions that are involved in several biological and pathological processes, and can be used in drug delivery.

Multipotent cardiovascular progenitor cells can be generated from stem cells or mesoderm cells. Stem cells are undifferentiated biological cells that can differentiate into specialized cells and can divide (through mitosis) to produce more stem cells. The stem cell can come from a human or a non-human organism (e.g., a mouse, a rat, or a pig). In some embodiments, the stem cell is not a human embryonic stem cell. Stem cells can also be obtained from differentiated cells by induced pluripotent stem cell (iPSC) technique. Thus, as used herein, stem cells include embryonic stem cell and induced pluripotent stem cells. Methods of obtaining induced pluripotent stem cells are known in the art, e.g., U.S. Pat. No. 8,058,065, US Pub. No. 2013/0130387, and US Pub. No. 2014/0093486, each of which is incorporated by reference herein in its entirety.

The methods described herein can effectively produce a sufficient number of multipotent cardiovascular progenitor cells for therapeutic and cardiac disease-modeling purposes. The number of cells can range, e.g., from about 1×10⁴ to about 1×10⁹, or from about 1×10⁵ to about 1×10⁷ cells. In some embodiments, the number of multipotent cardiovascular progenitor cells is over 10⁴, 10⁵, 10⁶, 10⁷, 10⁸, or 10⁹. In some embodiments, the number of multipotent cardiovascular progenitor cells is less than 10¹⁰, 10⁹, 10⁸, 10⁷, 10⁶, or 10⁵. The methods described here can also generate homogenous multipotent cardiovascular progenitor cells. In some embodiments, the percentage of multipotent cardiovascular progenitor cells among all cultured cells is over 50%, 60%, 70%, 80%, 90%, 95%, or 99%. The cells can be further sorted, e.g., by flow cytometry, to increase homogeneity.

The multipotent cardiovascular progenitor cells are useful for various purposes, e.g., therapeutic use (treating cardiovascular disorders), screening compounds for treating various disorders (e.g., cardiovascular disorders), and disease modeling etc.

### Inhibitory Nucleic Acids

The present disclosure also provides inhibitory nucleic acids for generating multipotent cardiovascular progenitors as described herein. In one aspect, the present disclosure provides methods of promoting multipotent cardiovascular progenitor cell formation. The methods involving contacting cells with Foxa2 and/or Tcf3 inhibitory nucleic acids (e.g., Foxa2 and Tcf3 siRNAs: siTcf3 and siFoxa2). In another aspect, the present disclosure provides methods of inhibiting multipotent cardiovascular progenitor cell formation, inhibiting mesoderm cell differentiation, or promoting endoderm cell generation. The methods involve contacting cells with Id1, Id2, Id3, Id4, Evx1, Grrp1, and/or Mesp1 inhibitory nucleic acids.

Inhibitory nucleic acids useful in the present methods and compositions include antisense oligonucleotides, ribozymes, external guide sequence (EGS) oligonucleotides, siRNA compounds, single- or double-stranded RNA interference (RNAi) compounds such as siRNA compounds, modified bases/locked nucleic acids (LNAs), peptide nucleic acids (PNAs), and other oligomeric compounds or oligonucleotide mimetics which hybridize to at least a portion of the target nucleic acid and modulate its function. In some embodiments, the inhibitory nucleic acids include antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

In some embodiments, the inhibitory nucleic acids are 10 to 50, 10 to 20, 10 to 25, 13 to 50, or 13 to 30 nucleotides in length. One having ordinary skill in the art will appreciate that this embodies inhibitory nucleic acids having complementary portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or any range therewithin.

The inhibitory nucleic acids useful in the present methods are sufficiently complementary to the target RNA, i.e., hybridize sufficiently well and with sufficient specificity, to give the desired effect. “Complementary” refers to the capacity for pairing, through hydrogen bonding, between two sequences comprising naturally or non-naturally occurring bases or analogs thereof. For example, if a base at one position of an inhibitory nucleic acid is capable of hydrogen bonding with a base at the corresponding position of a RNA, then the bases are considered to be complementary to each other at that position. 100% complementarity is not required.

Routine methods can be used to design an inhibitory nucleic acid that binds to the sequence of interest (e.g., Foxa2 or Tcf3 mRNA) with sufficient specificity. In some embodiments, the methods include using bioinformatics methods known in the art to identify regions of secondary structure, e.g., one, two, or more stem-loop structures, or pseudoknots, and selecting those regions to target with an inhibitory nucleic acid. For example, “gene walk” methods can be used to optimize the inhibitory activity of the nucleic acid; for example, a series of oligonucleotides of 10-30 nucleotides spanning the length of a target RNA can be prepared, followed by testing for activity. Optionally, gaps, e.g., of 5-10 nucleotides or more, can be left between the target sequences to reduce the number of oligonucleotides synthesized and tested. GC content is preferably between about 30-60%. Contiguous runs of three or more Gs or Cs should be avoided where possible (for example, it may not be possible with very short (e.g., about 9-10 nt)oligonucleotides).

In some embodiments, the inhibitory nucleic acid molecules can be designed to target a specific region of the RNA sequence. For example, a specific functional region can be targeted, e.g., a region comprising a known RNA localization motif (i.e., a region complementary to the target nucleic acid on which the RNA acts). Alternatively or in addition, highly conserved regions can be targeted, e.g., regions identified by aligning sequences from disparate species such as primate (e.g., human) and rodent (e.g., mouse) and looking for regions with high degrees of identity. Percent identity can be determined routinely using basic local alignment search tools (BLAST programs) (Altschul et al., J. Mol. Biol., 1990, 215, 403-410; Zhang and Madden, Genome Res., 1997, 7, 649-656), e.g., using the default parameters.

Once one or more target regions, segments or sites have been identified, e.g., within a sequence known in the art or provided herein, inhibitory nucleic acid compounds are chosen that are sufficiently complementary to the target, i.e., that hybridize sufficiently well and with sufficient specificity (i.e., do not substantially bind to other non-target RNAs), to give the desired effect.

It is understood in the art that a complementary nucleic acid sequence need not be 100% complementary to that of its target nucleic acid to be specifically hybridisable. A complementary nucleic acid sequence for purposes of the present methods is specifically hybridisable when binding of the sequence to the target RNA molecule interferes with the normal function of the target RNA to cause a loss of activity, and there is a sufficient degree of complementarity to avoid non-specific binding of the sequence to non-target RNA sequences under conditions in which specific binding is desired, e.g., under physiological conditions in the case of in vivo assays or therapeutic treatment, and in the case of in vitro assays, under conditions in which the assays are performed under suitable conditions of stringency.

In general, the inhibitory nucleic acids useful in the methods described herein have at least 80% sequence complementarity to a target region within the target nucleic acid, e.g., 90%, 95%, or 100% sequence complementarity to the target region within an RNA. For example, an antisense compound in which 18 of 20 nucleobases of the antisense oligonucleotide are complementary, and would therefore specifically hybridize, to a target region would represent 90 percent complementarity. Inhibitory nucleic acids that hybridize to an RNA can be identified through routine experimentation. In general, the inhibitory nucleic acids must retain specificity for their target, i.e., must not directly bind to, or directly significantly affect expression levels of, transcripts other than the intended target.

For further disclosure regarding inhibitory nucleic acids, see US Pub. No. 2010/0317718 (antisense oligos); US Pub. No. 2010/0249052 (double-stranded ribonucleic acid (dsRNA)); US Pub. No. 2009/0181914 and US Pub. No. 2010/0234451 (LNAs); US Pub. No. 2007/0191294 (siRNA analogues); US Pub. No. 2008/0249039 (modified siRNA); and WO2010/129746 and WO2010/040112 (inhibitory nucleic acids), each of which is incorporated by reference herein in its entirety.

### Cardiovascular Regeneration

The present disclosure also provides methods of promoting cardiac regeneration or treating cardiovascular disorders in a subject. The methods involve generating a plurality of multipotent cardiovascular progenitor cells from a plurality of stem cells, and delivering an effective amount of multipotent cardiovascular progenitor cells to the subject.

A subject to be treated according to the methods described herein is one who has suffered an injury or has an illness or disorder that in need of cardiovascular regeneration. In certain cases, the subject has a cardiovascular disorder, e.g., myocardial infarction, ischemic heart disease, hypertrophic cardiomyopathy, congenital cardiomyopathy, cardiac injury etc. The injury can be an infarction that results in tissue necrosis, or a mechanical injury. Tissue can be torn, crushed, scarred, weakened, or lost as a result of the mechanical injury. Scar tissue may have formed at the site of damage. The heart may be reduced in size due to loss of healthy cells as a result of ischemia or other disease. Other types of damage and disease can be treated as well. Such damage and disease can be ameliorated by repopulating or replacing the affected area by healthy cells of the appropriate cell type, or augmenting/expanding the healthy tissue by adding healthy cells of the appropriate cell type to the affected site.

Repopulating or replacing the affected area by healthy cells or augmenting/expanding the healthy tissue is accomplished by introducing multipotent cardiovascular progenitor cells to the site of damage or disease. In some embodiments, multipotent cardiovascular progenitor cells are generated from induced pluripotent stem cells derived from the cells of the subject. The use of autologous cells eliminates the risk of rejection of the implanted cell/tissue by the recipient.

Alternatively, multipotent cardiovascular progenitor cells can be generated from embryonic stem cells, or induced pluripotent stem cells derived from cells of a cell line, or cells that are obtained from a donor subject. When allogeneic or xenogeneic cells are used, it is preferred that the donor and the subject be FILA-compatible to the extent possible. When allogeneic or xenogeneic cells are used, methods of suppressing the immune system of the recipient can be used, including, but not limited to, the administration of immunosuppressive drugs, radiation, chemotherapeutics, or antibody masking techniques or agents.

The amount of multipotent cardiovascular progenitor cells that is administered to a subject can vary depending on the need of the subject. The effective amount of multipotent cardiovascular progenitor cells can be determined by observing the effects of the treatment. The composition comprising multipotent cardiovascular progenitor cells can include a mixed population of different subpopulations of cells, e.g., undifferentiated stem cells, undifferentiated mesoderm cells, multipotent cardiovascular progenitor cells, cardiomyocytes, endothelial cells etc. Separation methods (e.g., fluorescence-activated cell sorting) can be employed to enrich for multipotent cardiovascular progenitor cells.

The multipotent cardiovascular progenitor cells can be used the same day or cryogenically stored for later use. Cryogenic preservation methods are known in the art. The cells can also be expanded ex vivo using methods known in the art. The cells can also be subjected to other manipulations including the introduction of exogenous nucleic acids. Methods for introducing nucleic acids to mammalian cells are known in the art and include, but are not limited to, transfection, electroporation, lipofection, and other methods. Nucleic acids can be introduced prior to or following expamultipotent cardiovascular progenitor cell generation.

The multipotent cardiovascular progenitor cells can be administered to the subject, or delivered to the heart or the specific site of the organ, using any methods known in the art. For example, the cells can be delivered to the tissue by intramuscular or intramyocardial injection using a needle or other delivery device. Alternatively, the cells can be delivered by a catheter, such as a Stilleto catheter (Boston Scientific, Natick MA). The cells can also be delivered using surgical procedures, or during surgical procedures if appropriate; or they can be delivered by intracoronary infusion, intraarterial infusion, intravenous infusion, or retrograde perfusion. While non-surgical methods are preferred when possible, the route and method of introduction can vary depending on the tissue to be treated as well as the size of the damaged or diseased area. The cells can be delivered in a single procedure, or in more than one procedure. The number of cells delivered to the site of damage or disease can vary depending on the size of the damaged or diseased area and the severity of damage or disease progression. The number of cells can range, e.g., from about 1×10⁴ to about 1×10⁹, or from about 1×10⁵ to about 1×10⁷ cells. In some embodiments, the number of multipotent cardiovascular progenitor cells is over 10⁴, 10⁵, 10⁶, 10⁷, 10⁸, or 10⁹. In some embodiments, the number of multipotent cardiovascular progenitor cells is less than 10¹⁰, 10⁹, 10⁸, 10⁷, 10⁶, or 10⁵.

The cells can be included in formulations suitable for administration directly into tissues or organs, or suitable for administration into the bloodstream. A suitable formation can be determined by a medical practitioner according to standard procedures. Thus, a pharmaceutical composition can include an effective amount of multipotent cardiovascular progenitor cells and a suitable pharmaceutically acceptable carrier. Suitable pharmaceutically acceptable carriers and their formulation are known in the art (see, e.g., Remington's Pharmaceutical Sciences 16th edition, Osol, A. Ed. 1980). Cells are preferably formulated in solution at a pH from about 6.5 to about 8.5. Excipients to bring the solution to isotonicity can also be added, for example, 4.5% mannitol or 0.9% sodium chloride, pH buffered with art-known buffer solutions, such as sodium phosphate. Other pharmaceutically acceptable agents can also be used to bring the solution to isotonicity, including, but not limited to, dextrose, boric acid, sodium tartrate, propylene glycol, polyols (such as mannitol and sorbitol) or other inorganic or organic solutes.

In some embodiments, an agent that can promote a multipotent cardiovascular progenitor cell formation is administered to a subject to stimulate endogenous regeneration activity. The agent induces the formation of multipotent cardiovascular progenitors from the subject's own cells in the heart. The agent can be any proteins, nucleic acids, or compounds as described in the present disclosure, e.g., Id1, Id2, Id3, Id4, Evx1, Mesp1, and/or Grrp1 proteins, and/or nucleic acids encoding Id1, Id2, Id3, Id4, Evx1, Mesp1, and/or Grrp. In some embodiments, the agent is a modified RNA, a small molecule, RNA, protein, or inducers of Id1, Id2, Id3, Id4, Evx1, Mesp1, and/or Grrp1. In some embodiments, the agent is delivered through viral delivery, exosomal delivery, etc.

In some embodiments, an agent that can promote a multipotent cardiovascular progenitor cell formation can induce the formation of multipotent cardiovascular progenitors from the cells (e.g., stem cells) that are delivered to the subject. Thus, the agent can be delivered prior to, during, or after the cells are delivered to a subject. In some embodiments, the cells and/or the agents are delivered directly to heart.

### Methods of Screening

Included herein are methods for screening test compounds, e.g., polypeptides, polynucleotides, inorganic or organic large or small molecule test compounds, to identify agents useful in promoting multipotent cardiovascular progenitor cell formation. In some embodiments, the cells can be ventricular-like cardiac cells and/or atrial-like cardiac cells.

As used herein, “small molecules” refers to small organic or inorganic molecules of molecular weight below about 3,000 Daltons. In general, small molecules useful for the methods described herein have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

The test compounds can be, e.g., natural products or members of a combinatorial chemistry library. In some embodiments, the test compound is a small molecule, a nucleic acid, a peptide, a protein, an oligonucleotide, an antisense molecule, a small interfering RNA, a small hairpin RNA, an antibody or an antigen-binding fragment.

A set of diverse molecules can be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in some embodiments, a general library of small molecules is screened, e.g., using the methods described herein.

In some embodiments, a test compound is applied to a test sample, e.g., a mesoderm cell, a stem cell (e.g., an embryonic stem cell or an induced pluripotent stem cell), a cultured cell from a cell line. One or more effects of the test compound is evaluated. In a cultured cell for example, the ability of the test compound to generate multipotent cardiovascular progenitor cells is evaluated. The multipotent cardiovascular progenitor cells can be determined by mesoderm-specific markers (e.g., Kdr, Mesp1, Snai1, Cdh11). In some embodiments, the ability of the test compound to increase expression or activity of one or more proteins selected from the group consisting of Id1, Id2, Id3, Id4, Evx1, Grrp1, and Mesp1 is evaluated. In some embodiments, the ability of the test compound to decrease expression or activity of Foxa2 and/or Tcf3 is evaluated.

In some embodiments, the test sample is, or is derived from (e.g., an induced pluripotent stem cell derived from) a subject having cardiovascular disorders (e.g., myocardial infarction, ischemic heart disease, hypertrophic cardiomyopathy, or congenital cardiomyopathy) or an in vivo model for cardiovascular disorders. The in vivo model can be an animal model, for example, a rodent such as a rat or a mouse can be used.

Methods for evaluating each of these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on gene expression level (e.g., the expression level of Id1, Id2, Id3, Id4, Evx1, Grrp1, Mesp1, Foxa2 and/or Tcf3).

A test compound that has been screened by a method described herein and determined to promote multipotent cardiovascular progenitor cell formation, can be considered a candidate agent. Candidate agents, once screened in a clinical setting, are therapeutic agents. Therapeutic agents (e.g., small molecules) can be optionally optimized and/or derivatized, and formulated with physiologically acceptable excipients to form pharmaceutical compositions.

Thus, test compounds identified as “hits” (e.g., test compounds that can promote multipotent cardiovascular progenitor cell formation) in a first screen can be selected and systematically altered, e.g., using rational design, to optimize binding affinity, avidity, specificity, or other parameter. Such optimization can also be screened for using the methods described herein. Thus, in some embodiments, the methods include screening a first library of compounds using a method known in the art and/or described herein, identifying one or more hits in that library, subjecting those hits to systematic structural alteration to create a second library of compounds structurally related to the hit, and screening the second library using the methods described herein.

Test compounds identified as hits can be considered candidate therapeutic compounds, useful for generating multipotent cardiovascular progenitor cells. A variety of techniques useful for determining the structures of “hits” can be used in the methods described herein, e.g., NMR, mass spectrometry, gas chromatography equipped with electron capture detectors, fluorescence and absorption spectroscopy. Thus, this disclosure also provides compounds identified as “hits” by the methods described herein, and methods of using the “hits” for generating multipotent cardiovascular progenitor cells.

### Assessing Cardiac Toxicity

A large percentage of new drugs failing in clinical studies due to cardiac toxicity. Thus, determining cardiac toxicity is important for predicting the side effects of drugs. The multipotent cardiovascular progenitor cells can be used to assess cardiac toxicity of test compounds. In one aspect, the methods involve contacting multipotent cardiovascular progenitor cells with a test compound, analyzing a plurality of cellular metabolites, and comparing cellular metabolites from multipotent cardiovascular progenitor cells contacted with the test compound to cellular metabolites of multipotent cardiovascular progenitor cells not contacted with the test compound. If the cellular metabolites comprise a metabolic profile characteristic of multipotent cardiovascular progenitor cells in response to a cardiotoxic compound, the test compound will be determined to have cardiac toxicity.

The multipotent cardiovascular progenitor cells can further differentiate into cardiomyocytes. Many methods are known in the art to assess cardiac toxicity of test compounds in cardiomyocytes. For example, cardiac toxicity to cardiomyocytes can be determined by analyzing cellular metabolites, monitoring cardiomyocyte contractions using a calcium sensitive dye, and cardiac beating assay, etc. These methods are described, e.g., in WO2011044253, WO2010094757, U.S. Pat. No. 9,624,471, US Pub. No. 2011/0318775, and Liang et al. “Drug screening using a library of human induced pluripotent stem cell-derived cardiomyocytes reveals disease specific patterns of cardiotoxicity,” Circulation (2013): CIRCULATIONAHA.113.001883, each of which is incorporated by reference herein in its entirety.

### Disease Modeling and Tissue Engineering

The multipotent cardiovascular progenitor cells are useful for various purposes, e.g., therapeutic use (treating cardiovascular disorders), screening compounds for treating various disorders (e.g., cardiovascular disorders), toxicology studies, tissue engineering, and disease modeling etc. Disease models can be developed using single cell types for various diseases (e.g. channelopathies, myopathies) or multiple cell types to model more complex disease phenotypes (e.g. vasculopathies). Disease models can be used for drug screening, developing personalized medicine (i.e. developing individualized treatments) and discovering basic disease mechanisms.

Multipotent cardiovascular progenitor cells can be used in disease modeling, e.g., for various cardiovascular diseases. Cardiovascular diseases include, e.g., coronary artery diseases (CAD) such as angina and myocardial infarction (commonly known as a heart attack), stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis. In some embodiments, these multipotent cardiovascular progenitor cells can be directly used in the disease modeling. In some embodiments, these multipotent cardiovascular progenitor cells can be differentiated into appropriate cell types (e.g., ventricular-like cardiac cells, atrial-like cardiac cells, cardiogenic progenitors, skeletal muscle cells, vascular cells, or fibroblasts). These disease models can also be used to screen for drugs that can be used to treat these diseases. The multipotent cardiovascular progenitor cells can also carry genetic mutations of interest. These genetic mutations can be introduced into cells by methods known in the art, e.g., CRISPR/Cas9 targeted genome editing. Screening can be performed to identify compounds that can mitigate or reverse the effects of the genetic mutations.

The multipotent cardiovascular progenitor cells can also be differentiated into appropriate cell types, and be used in tissue engineering. Appropriate cell types include, e.g., cardiogenic progenitors, skeletal muscle cells, vascular cells, fibroblasts. Engineered tissues using such cells can be used as tools for drug screening and discovery, be used in diagnostics and prognostic tools, and be used to personalize treatment options. They can also be used for regenerative applications, as described above. In some embodiments, these cells are skeletal muscle cells and can be used in skeletal muscle regeneration. In some embodiments, these cells are cardiogenic progenitors and can be used in treating various cardiovascular diseases.

### Pharmaceutical Compositions

The methods described herein include the use of pharmaceutical compositions comprising multipotent cardiovascular progenitor cells or various agents or compounds that are described in this disclosure (e.g., compounds that are identified by the screening methods or compounds that are evaluated for cardiac toxicity).

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration.

Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, NJ) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In some embodiments, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

### Example 1: Materials and Methods

The following materials and methods were used in the following examples.

mESC Culture

mESCs Kdr-eGFP (Ema et al., 2006) were maintained in DMEM High Glucose (HyClone, Logan, Utah) medium supplemented with 10% fetal bovine serum, Sodium Pyruvate 1 mM (Sigma, St. Louis, MO), MEM NEAA 1× (Gibco, Grand Island, NY), L-Glutamine 2 mM (Gibco, Grand Island, NY), Penicillin-Streptomycin 100 units/mL and 100 Kg/mL (HyClone, Logan, Utah), (3-mercaptoethanol 50 μM (Sigma, St. Louis, MO) and LIF 1000 units/mL (Millipore, Billerica, MA). For differentiation, mESCs were seeded in 10 cm low-attachment tissue culture dishes at a density of 10⁶ cells/dish in a chemically-defined media (CDM) (Gadue et al., 2006) where they formed embryoid bodies (EBs) over a period of 2 days. At day 2, EBs were then dissociated using 0.25% Trypsin EDTA (Gibco, Grand Island, NY), washed in PBS and replated in CDM supplemented with 50 ng/mL of Recombinant Human Activin A (338-AC-050, R&D Systems, Minneapolis, MN) in 10 cm low-attachment tissue culture dishes.

### Kdr-eGFP Assay

On day 3 of differentiation, EBs were collected and dissociated using 0.25% Trypsin EDTA; 10⁴ cells/well were plated in 100 μL of CDM containing Recombinant Human Activin A (50 ng/mL) into gelatin coated 384-well optical tissue culture plates (Greiner Bio-One, Kremsmunster, Austria), pre-spotted with 25 nM siRNAs in 0.2 μL Lipofectamine RNAiMax+14.8 μL OPTI-MEM I (Gibco, Grand Island, NY). Fixation was performed at day 6 using 4% Paraformaldehyde. Next, wells are imaged using HT microscope (ImageXPress, Molecular Devices, Sunnyvale, CA) and fluorescence is quantified using custom method developed in MetaXpress Analysis software (Molecular Devices, Sunnyvale, CA) to determine integrated pixel intensity of Kdr-eGFP.

hESC Culture

Cells were routinely maintained in mTeSR1 media (05850, Stem Cell Technologies, Vancouver, Canada) on growth factor-reduced Matrigel (9 μg/cm²) and passaged every 4 days using ReLeSR (05872, Stem Cell Technologies, Vancouver, Canada). H9 hESC lines (WA09) were supplied by WiCell Research Institute. H9 hESCs were cultured for at least five passages before beginning differentiation. Cells were maintained with 2.5 mL medium per 9.6 cm² of surface area, or equivalent. All pluripotent cultures were routinely tested for mycoplasma contamination using a MycoAlert Kit (Lonza, Basel, Switzerland).

### Lentivirus Preparation

Large-scale lentivirus production was performed. Three plasmids including lentivector, pCMVDR8.74, and pMD2. G were co-transfected into HEK-293T cells in a ratio of 3:2:1. UltraCULTURE™ serum-free medium (Lonza, Basel, Switzerland) supplemented with 1 mM L-glutamine (Life Technologies, Carlsbad, CA) was used to re-feed transfected cells and the supernatant was collected every 24 hours from day 2 to day 4 post-transfection. All viral supernatant was pooled and filtrated through 0.22 μm pores, followed by concentration and purification using 20% sucrose gradient ultra-centrifugation at 21,000 rpm for 2 hours. The pellet containing concentrated viral particles was resuspended in PBS, aliquoted, and kept in −80° C. for long term storage.

Generation of Transgenic Cell Lines (mESCs and hESCs)

The following modifications were applied to pCDH-CMV vector (Cat #CD511B-1, System Biosciences, Palo Alto, CA): the CMV promoter driving the expression of the MCS was replaced by the Ef1a promoter to ensure robust expression in ESC stages, and the Ef1a-CopGFP cassette was replaced by a pgk-puro cassette to enrich for infected clones.

mESCs with Kdr-eGFP (Ema et al. 2006) were infected with all possible combinations of high-titer lentiviruses (modified pCDH-CMV) overexpressing Id1, Evx1, or Grrp1 and subsequently grown under continuous puromycin selection (2 μg/mL) (227420100, Acros, Geel, Belgium).

Similarly, H9 hESCs were infected with Id1-overxpressing lentivirus and selected with puromycin 6 μg/mL.

### Mouse Id1-Induced MCPs

Id1-overexpressing mESCs were grown and differentiated as wild type mESCs, in the presence of 2 μg/mL puromycin. At day 3, cells were collected and dissociated with 0.25% Trypsin EDTA (Gibco, Grand Island, NY), trypsin was inactivated with 10% FBS-containing media, cells were washed in PBS and resuspended in CDM supplemented with Recombinant Human Activin A (300 ng/ml)+puromycin (2 μg/mL). 10⁷ cells were replated onto a 15 cm gelatin coated tissue culture dish into 30 mL of CDM+Recombinant Human Activin A (300 ng/mL)+puromycin (2 μg/mL) and cultured for 3 days. At day 6, cells were collected and frozen in freezing media (10% DMSO, 20% FBS, 70% DMEM High-glucose (HyClone, Logan, Utah)) at a density of 3-5×10⁶ cells per vial and stored in liquid nitrogen.

### Human Id1-Induced MCPs

hESCs were dissociated using 0.5 mM EDTA (Life Technologies, Carlsbad, CA) in PBS without CaCl₂ or MgCl₂ (21-040-CV, Corning, Corning, NY) for 7 minutes at room temperature. Cells were plated at 3×10⁵ cells per well of a 12 well plate in mTeSR1 media (Stem Cell Technologies, Vancouver, Canada) supplemented with 2 μM Thiazovivin (Selleck Chemicals, Houston, TX) for the first 24 h after passage. Cells were fed daily for 3-5 days until they reached 2′ 90% confluence, whereby they were washed with PBS and the medium was changed to basal differentiation media (BDM), consisting of RPMI 1640 medium (11875-093, Life Technologies, Carlsbad, CA) and B27 Supplement minus insulin (A1895601, Life Technologies, Carlsbad, CA). For the first 24 hour differentiation period, the BDM media was supplemented with 300 ng/mL Recombinant Human Activin A, and 2 μg/mL of Puromycin (227420100, Acros, Geel, Belgium). After 24 hours this l medium was replaced with basic BDM supplemented with 6 μg/ml of puromycin. BDM+puromycin (2 μg/mL) was replaced every 48 hours. At day 5, cells were collected and frozen for later use.

### Spontaneous of Cryopreserved Id1-Induced MCPs

To resume differentiation, MCPs (mouse or human) were thawed in 37° C. water bath for 3 minutes, washed and resuspended in BDM+ hES cell recovery supplement (2 μM) (Stemgent, Cambridge, MA) for human Id1-induced MCPs or CDM+ hES cell recovery supplement (2 μM) for mouse-induced MCPs and plated onto gelatin coated 384-well culture plates (Greiner Bio-One, Kremsmunster, Austria) at a cell density of 25,000 cells/well. Media (BDM or CDM) was replaced every other day until day 15 of differentiation.

siRNAs

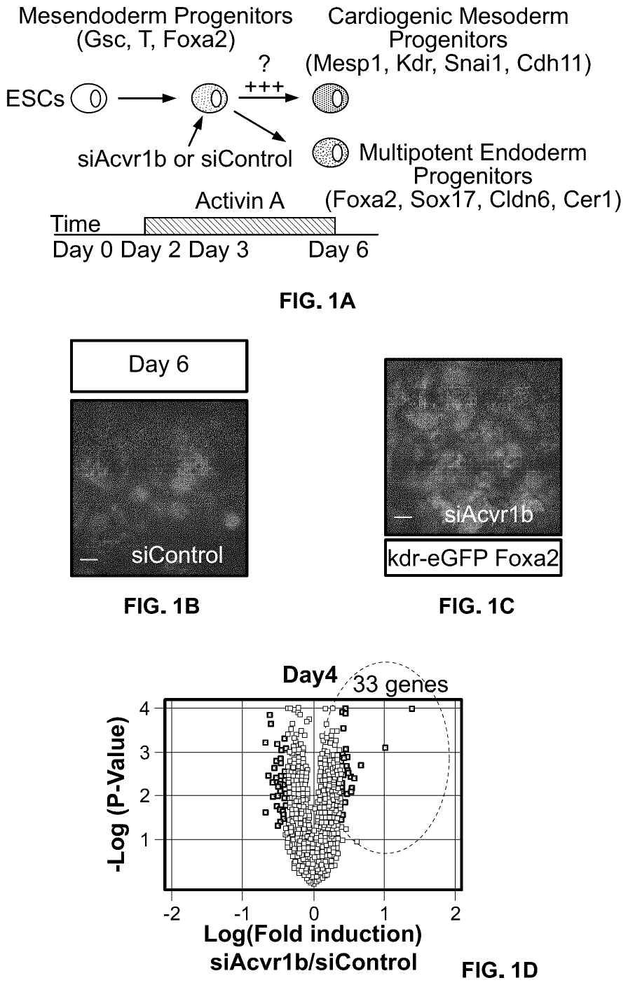

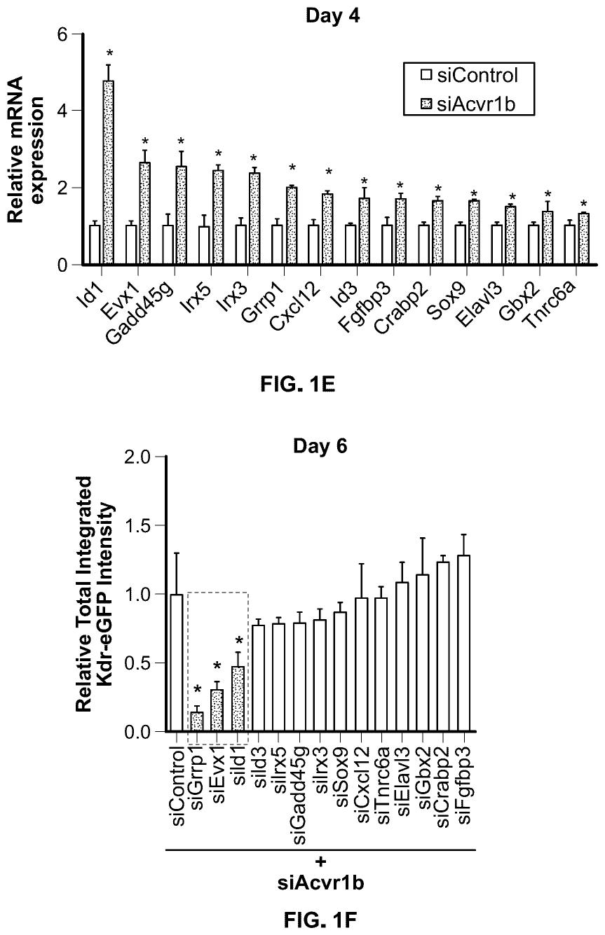

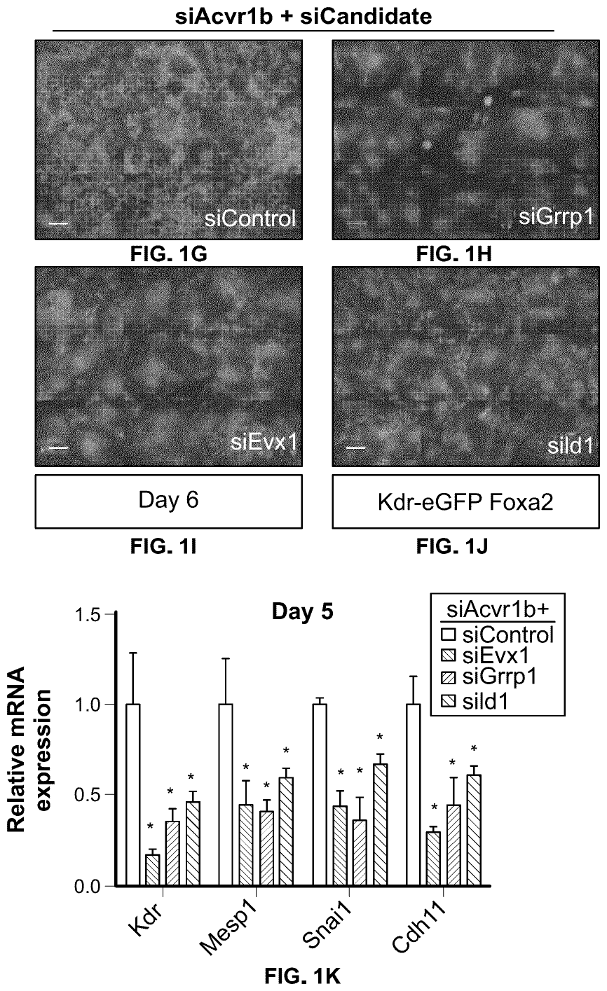

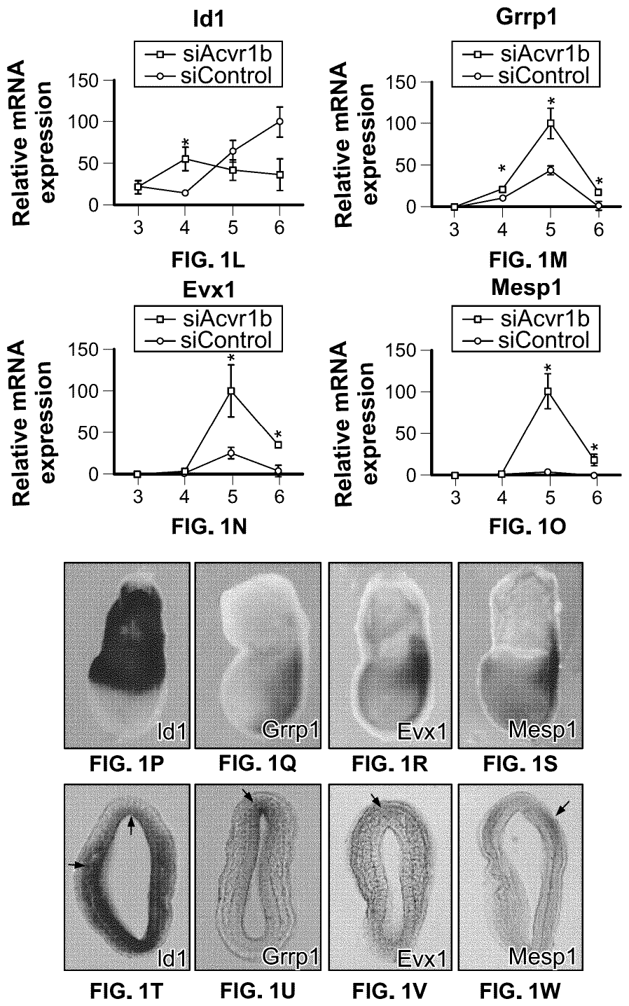

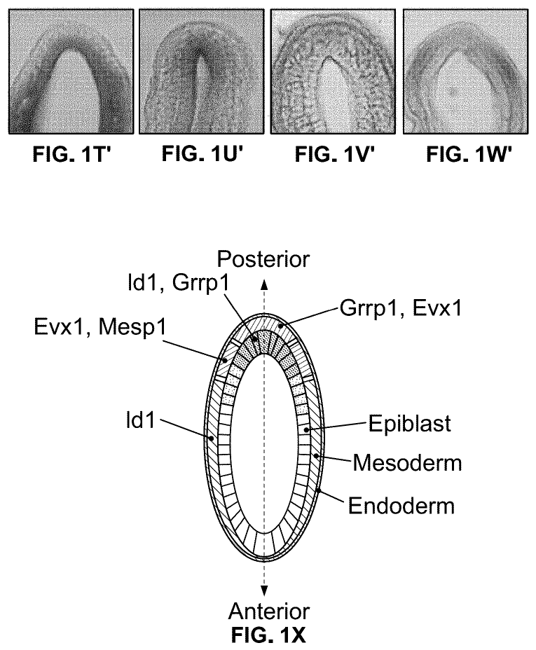

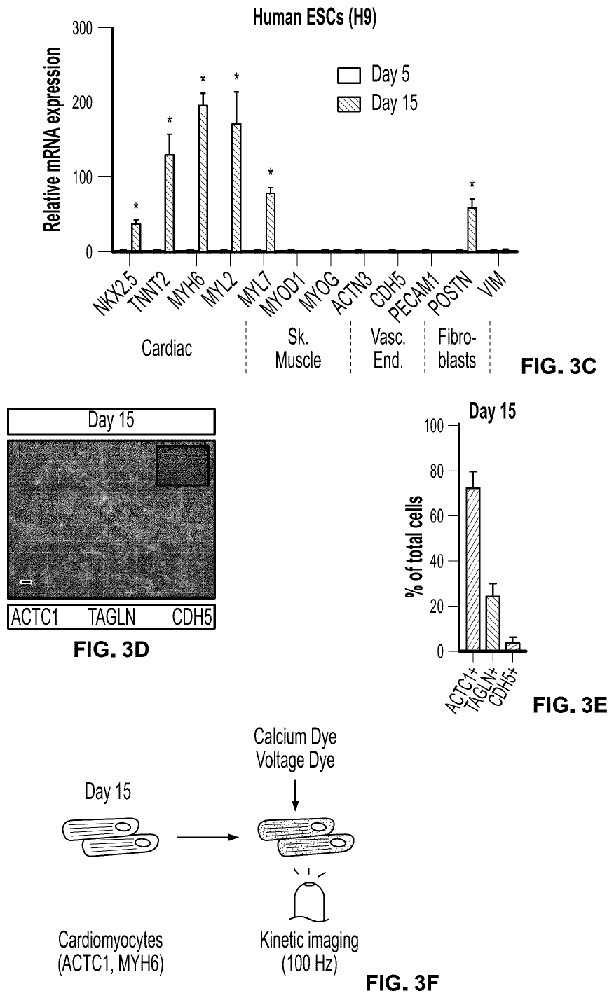

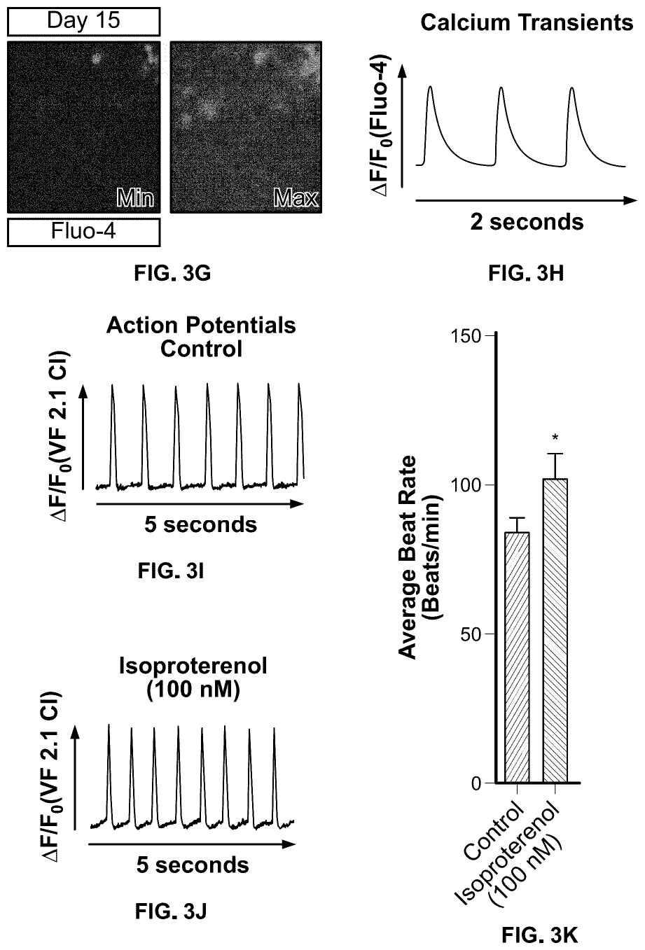

siRNAs from FIG. 1F and FIG. 3F were cherry-picked from mouse genome-wide siGENOME SMARTpool library from Dharmacon (Lafayette, CO) and were transfected at a final concentration of 12.5 nM. All remaining siRNAs were purchased from life technologies (Silencer™ select siRNAs) and transfected at a final concentration of 25 nM: siControl (AM4611), siEvx1 (s65742), siFoxa2 (s67627), siGrrp1 (s91214), siId1 (s68006), siTcf3 (s74856), siTcf4 (s74829), siTcf12 (s74811).

### Immunostaining for Cell Culture and Cardiovascular Lineage Quantification

Cells grown on gelatin coated 384-well plates (Greiner Bio-One, Kremsmunster, Austria) were fixed using 4% paraformaldehyde and immunostained by incubating in block solution (10% horse serum, 0.5% Triton X100, and 0.01% gelatin in phosphate buffered saline (PBS)) for 30 minutes at room temperature followed by incubation with antibodies directed against Foxa2 (sc-6554, Santa Cruz Biotechnology, Dallas, TX), Pecam1 (sc-1506, Santa Cruz Biotechnology, Dallas, TX), Actc1 (A7811, Sigma, St. Louis, MO), CDH5 (AF938, R&D Systems, Minneapolis, MN) for 1 hour at room temperature in the block solution. The cells were then washed 3 times with PBS and incubated with Alexa-conjugated secondary antibodies (Life Technologies, Carlsbad, CA) in block solution at room temperature for 1 hour. The cells were then washed 3 times with PBS, and stored in 50% glycerol (v/v) in PBS. Next, wells were imaged using HT microscope (ImageXPress, Molecular Devices, Sunnyvale, CA) and fluorescence was quantified using custom method developed in MetaXpress Analysis software (Molecular Devices, Sunnyvale, CA) to determine the % of ACTC1, TAGLN and CDH5 positive cells.

### Reverse Transcription Quantitative PCR Analysis (RT-qPCR)

Total RNA was extracted with miRVana isolation kit (AM1540, Ambion, Waltham, MA) and reverse transcribed to cDNA with QuantiTect Reverse Transcription Kit (Qiagen, Hilden, Germany) according to the manufacturer's instructions. cDNA samples synthesized from 1 μg of total RNA were subjected to RT-qPCR with 7900HT Fast Real-Time PCR System (Applied Biosystems, Foster City, CA) using the iTaq SYBR Green Supermix with ROX (Bio-Rad). Primer sequences are listed in Table 1. The data was analyzed with the ΔΔCt method applying β-Actin as a normalization control.

TABLE 1
Quantitative PCR oligonucleotide primers
Mouse primers

| Gene Name | Gene Bank Accession | Sequence |
|---|---|---|
| Acrv1b | NM_007395 | F: TTCTTCCCCCTTGTTGTCCTC (SEQ ID NO: 25) |
| | | R: ACAGGTGTAGTTGGTCTGTAGG (SEQ ID NO: 26) |
| Actc1 | NM_009608 | F: CTGGATTCTGGCGATGGTGTA (SEQ ID NO: 27) |
| | | R: CGGACAATTTCACGTTCAGCA (SEQ ID NO: 28) |
| Actn3 | NM_013456 | F: AACAGCAGCGGAAAACCTTCA (SEQ ID NO: 29) |
| | | R: GGCTTTATTGACATTGGCGATTT (SEQ ID NO: 30) |
| Cdh11 | NM_009866 | F: CTGGGTCTGGAACCAATTCTTT (SEQ ID NO: 31) |
| | | R: GCCTGAGCCATCAGTGTGTA (SEQ ID NO: 32) |
| Cdh5 | NM_009868 | F: CACTGCTTTGGGAGCCTTC (SEQ ID NO: 33) |
| | | R: GGGGCAGCGATTCATTTTTCT (SEQ ID NO: 34) |
| Chst15 | NM_029935 | F: TTCCCCGAAGACACACACAAA (SEQ ID NO: 35) |
| | | R: CCCCAGTTTTCATTGCCCTCA (SEQ ID NO: 36) |
| Crabp2 | NM_0007759 | F: ATGCCTAACTTTTCTGGCAACT (SEQ ID NO: 37) |
| | | R: GCACAGTGGTGGAGGTTTTGA (SEQ ID NO: 38) |
| Cxcl12 | NM_001012477 | F: TGCATCAGTGACGGTAAACCA (SEQ ID NO: 39) |
| | | R: TTCTTCAGCCGTGCAACAATC (SEQ ID NO: 40) |
| Dnajc6 | NM_001164584 | F: TGAAAATAAAGGTGCCTCGTCTC (SEQ ID NO: 41) |
| | | R: TCAGGTTACTGAATAGCCTCCC (SEQ ID NO: 42) |
| Egr1 | NM_007913 | F: TCGGCTCCTTTCCTCACTCA (SEQ ID NO: 43) |
| | | R: CTCATAGGGTTGTTCGCTCGG (SEQ ID NO: 44) |
| Elavl3 | NM_010487 | F: TCCTATGCACGTCCCAGTTCT (SEQ ID NO: 45) |
| | | R: TCGATCCTCTTGTCAAAGCGG (SEQ ID NO: 46) |
| Evx1 | NM_007966 | F: GAGAGCCGAAAGGACATGGTT (SEQ ID NO: 47) |
| | | R: CTGCCTGCTAGTCCATCGAC (SEQ ID NO: 48) |
| Fgf8 | NM_001166361 | F: CCGAGGAGGGATCTAAGGAAC (SEQ ID NO: 49) |
| | | R: CTTCCAAAAGTATCGGTCTCCAC (SEQ ID NO: 50) |
| Fgfbp3 | NM_028263 | F: GGTCGCTTCGTGAGTCCAG (SEQ ID NO: 51) |
| | | R: AGCAGCCGTCTCCAGTAGT (SEQ ID NO: 52) |
| FoxA2 | NM_010446 | F: CCCTACGCCAACATGAACTCG (SEQ ID NO: 53) |
| | | R: GTTCTGCCGGTAGAAAGGGA (SEQ ID NO: 54) |
| Gadd45g | NM_011817 | F: GGGAAAGCACTGCACGAACT (SEQ ID NO: 55) |
| | | R: AGCACGCAAAAGGTCACATTG (SEQ ID NO: 56) |
| Gbx2 | NM_010262 | F: CAACTTCGACAAAGCCGAGG (SEQ ID NO: 57) |
| | | R: ACTCGTCTTTCCCTTGCCCT (SEQ ID NO: 58) |
| Gemin6 | NM_026053 | F: GCCAACATTGTCCTCGTAAACT (SEQ ID NO: 59) |
| | | R: TGTGGTCCCCTTCACTTATGG (SEQ ID NO: 60) |
| Grm6 | NM_173372 | F: GCAGAAACATCTGGTTTGCTG (SEQ ID NO: 61) |
| | | R: CCTCCTGTTCATAGGTGGAGTC (SEQ ID NO: 62) |
| Grrp1 | NM_001099296 | F: AGGGACCACTGCAACTCAG (SEQ ID NO: 63) |
| | | R: CCATACACAGTTAAGGACGCAC (SEQ ID NO: 64) |
| Gsc | NM_010351 | F: CAGATGCTGCCCTACATGAAC (SEQ ID NO: 65) |
| | | R: TCTGGGTACTTCGTCTCCTGG (SEQ ID NO: 66) |
| Id1 | NM_010495 | F: CCTAGCTGTTCGCTGAAGGC (SEQ ID NO: 67) |
| | | R: CTCCGACAGACCAAGTACCAC (SEQ ID NO: 68) |
| Id3 | NM_008321 | F: CGACCGAGGAGCCTCTTAG (SEQ ID NO: 69) |
| | | R: GGACGCGATAGGGAAGACC (SEQ ID NO: 70) |
| Irx3 | NM_001253822 | F: TCTGGGTCCCTATCCAATGTG (SEQ ID NO: 71) |
| | | R: GGTCCCCGAACTGGTACTG (SEQ ID NO: 72) |
| Irx5 | NM_018826 | F: TACAGCACCAGCGTCATTTCG (SEQ ID NO: 73) |
| | | R: GAGCCCACGTAAGAGAAGGC (SEQ ID NO: 74) |
| Kdr | NM_010612 | F: TTTGGCAAATACAACCCTTCAGA (SEQ ID NO: 75) |
| | | R: GCAGAAGATACTGTCACCACC (SEQ ID NO: 76) |
| Lefty1 | NM_010094 | F: CCAACCGCACTGCCCTTAT (SEQ ID NO: 77) |
| | | R: CGCGAAACGAACCAACTTGT (SEQ ID NO: 78) |
| Lefty2 | NM_177099 | F: CAGCCAGAATTTTCGAGAGGT (SEQ ID NO: 79) |
| | | R: CAGTGCGATTGGAGCCATC (SEQ ID NO: 80) |
| Mesp1 | NM_008588 | F: GTCACTCGGTCCTGGTTTAAG (SEQ ID NO: 81) |
| | | R: ACGATGGGTCCCACGATTCT (SEQ ID NO: 82) |
| Myh6 | NM_010856 | F: GCCCAGTACCTCCGAAAGTC (SEQ ID NO: 83) |
| | | R: GCCTTAACATACTCCTCCTTGTC (SEQ ID NO: 84) |
| Myog | NM_031189 | F: GAGACATCCCCCTATTTCTACCA (SEQ ID NO: 85) |
| | | R: GCTCAGTCCGCTCATAGCC (SEQ ID NO: 86) |
| Nodal | NM_013611 | F: TTCAAGCCTGTTGGGCTCTAC (SEQ ID NO: 87) |
| | | R: TCCGGTCACGTCCACATCTT (SEQ ID NO: 88) |
| Pecam1 | NM_001032378 | F: ACGCTGGTGCTCTATGCAAG (SEQ ID NO: 89) |
| | | R: TCAGTTGCTGCCCATTCATCA (SEQ ID NO: 90) |
| Pitx2 | NM_011098 | F: GCAGCCGTTGAATGTCTCTTC (SEQ ID NO: 91) |
| | | R: GTCCGTGAACTCGACCTTTTT (SEQ ID NO: 92) |
| Snail | | F: CACACGCTGCCTTGTGTCT (SEQ ID NO: 93) |
| | | R: GGTCAGCAAAAGCACGGTT (SEQ ID NO: 94) |
| Sox9 | NM_011448 | F: GAGCCGGATCTGAAGAGGGA (SEQ ID NO: 95) |
| | | R: GCTTGACGTGTGGCTTGTTC (SEQ ID NO: 96) |
| Stk4 | NM_021420 | F: TCATTCGGCTACGGAACAAGA (SEQ ID NO: 97) |
| | | R: GACCTGCGACTCCAAAGTCTG (SEQ ID NO: 98) |
| Tnnt2 | NM_001130181 | F: CAGAGGAGGCCAACGTAGAAG (SEQ ID NO: 99) |
| | | R: CTCCATCGGGGATCTTGGGT (SEQ ID NO: 100) |
| Tnrc6a | NM_144925 | F: ATGCTCCTGAAAGCAAACCAG (SEQ ID NO: 101) |
| | | R: CCTTTTAGGGCAAGTCCATTGT (SEQ ID NO: 102) |
| Trim67 | NM_198632 | F: CCACTCTCTGCGAGCAATG (SEQ ID NO: 103) |
| | | R: GGTGGCTGAACTAGCCGAT (SEQ ID NO: 104) |
| Zfp750 | NM_178763 | F: ATGAGTCTCCTAAAGGAACGGA (SEQ ID NO: 105) |
| | | R: GGGAATACGATCTTGCTCTGAC (SEQ ID NO: 106) |
| Zmpste24 | NM_172700 | F: GCATCGGTGGACGCTATGT (SEQ ID NO: 107) |
| | | R: TGTGCTAGGAAGGTCTCCCAA (SEQ ID NO: 108) |

Human primers

| Gene Name | Gene Bank Accession | Sequence |
|---|---|---|
| ACTN3 | NM_001104 | F: GATGACCCCATCGGAAACCTG (SEQ ID NO: 109) |
| | | R: CTTGCAGATCCTGTTGGCAG (SEQ ID NO: 110) |
| CDH11 | NM_001797 | F: GTATCCTCGAAGGACAACCCT (SEQ ID NO: 111) |
| | | R: GACATCGGTCAGTGTGATCGT (SEQ ID NO: 112) |
| CDH5 | NM_001795 | F: AAGCGTGAGTCGCAAGAATG (SEQ ID NO: 113) |
| | | R: TCTCCAGGTTTTCGCCAGTG (SEQ ID NO: 114) |
| EVX1 | NM_001989 | F: GACCAGATGCGTCGTTACCG (SEQ ID NO: 115) |
| | | R: GTGGTTTCCGGCAGGTTTAG (SEQ ID NO: 116) |
| GRRP1 | NM_024869 | F: TCAAGACGCACCAGGTGATAG (SEQ ID NO: 117) |
| | | R: CGGTAGAAGATGAGGGAATCAGG (SEQ ID NO: 118) |
| ID1 | NM_181353 | F: CTGCTCTACGACATGAACGG (SEQ ID NO: 119) |
| | | R: GAAGGTCCCTGATGTAGTCGAT (SEQ ID NO: 120) |
| KDR | NM_002253 | F: GTGATCGGAAATGACACTGGAG (SEQ ID NO: 121) |
| | | R: CATGTTGGTCACTAACAGAAGCA (SEQ ID NO: 122) |
| MESP1 | | F: CCACCGTCCCCGCTCCTTCC (SEQ ID NO: 123) |
| | | R: CGGTGCTCACAGAGACGGCG (SEQ ID NO: 124) |
| MYH6 | NM_002471 | F: GCTGGTCACCAACAATCCCTA (SEQ ID NO: 125) |
| | | R: CGTCAAAGGCACTATCGGTGG (SEQ ID NO: 126) |
| MYOG | NM_002479 | F: GGGGAAAACTACCTGCCTGTC (SEQ ID NO: 127) |
| | | R: AGGCGCTCGATGTACTGGAT (SEQ ID NO: 128) |
| PECAM1 | NM_000442 | F: CCAAGGTGGGATCGTGAGG (SEQ ID NO: 129) |
| | | R: TCGGAAGGATAAAACGCGGTC (SEQ ID NO: 130) |
| SNAl1 | NM_005985 | F: TCGGAAGCCTAACTACAGCGA (SEQ ID NO: 131) |
| | | R: AGATGAGCATTGGCAGCGAG (SEQ ID NO: 132) |
| TNNT2 | NM_001001431 | F: ACAGAGCGGAAAAGTGGGAAG (SEQ ID NO: 133) |
| | | R: TCGTTGATCCTGTTTCGGAGA (SEQ ID NO: 134) |

### Microarray Experiment and Analysis

siControl or siAcvr1b were transfected in day 3 differentiating mESCs. Total RNA (500 ng) was collected at day 4 and hybridized on MouseRef-8 v2.0 Expression BeadChip (25,600 transcripts, Illumina, San Diego, CA). BeadChips were subsequently washed and developed with fluorolink streptavidin-Cy3 (GE Healthcare, Marlborough, MA). BeadChips were scanned with an Illumina BeadArray Reader, and hybridization efficiency was monitored using BeadStudio software (Illumina, San Diego, CA) to measure internal controls built into the Illumina system. Linear models were fitted for each gene using the Bioconductor limma package in R. Moderated t-statistics, fold-change and the associated P-values were calculated for each gene. To account for testing thousands of genes, false discovery rate (FDR)-adjusted values were calculated using the Benjamini-Hochberg method.

### Flow Cytometry

For live Kdr-eGFP cells, cells were dissociated using 0.25% Trypsin EDTA, blocked with 10% FBS-containing media and resuspended in PBS containing 0.5% FBS (washing buffer) for flow sorting using LSRFortessa or FACSAria Flow cytometers (BD Biosciences, San Jose, CA). For hESCs, day 5 cells dissociated using 1× TrypLE Express (Gibco, Grand Island, NY), blocked and washed with PBS containing 0.5% FBS (washing buffer). Cells were incubated for 20 minutes with PE anti-human CD309 (cat #359903, dilution 1:100, BioLegend, San Diego, CA) in PBS containing 0.5% FBS at 4° C. Next, cells were washed in washing buffer, fixed for 20 min in 1% PBS: formaldehyde at 4° C., washed and resuspended in washing buffer and processed by flow sorting.

### Xenopus Laevis Embryo Culture

Embryos were fertilized in vitro, dejellied in 2% cysteine-HCl, pH=7.8, and maintained in 0.1×MMR (Peng, 1991). Embryos were staged according to Nieuwkoop and Faber (Nieuwkoop, 1967). For gene expression analysis, whole embryos were fixed in MEMFA for in situ hybridization as below.

mRNA Injection in Xenopus Laevis

Synthetic capped mRNAs for Xid2 injection were transcribed from pSP64T plasmid using SP6 mMessage kit (Ambion, Waltham, MA). mRNAs were injected at 125 ng/blastomere at 4-cell stage embryos.

In Situ Hybridization in Xenopus laevis Embryos

In situ hybridization for Xbra (Colas et al., 2008), Xmespb was carried out as described, e.g., in Djiane et al., “Role of frizzled 7 in the regulation of convergent extension movements during gastrulation in Xenopus laevis,” Development, 127(14): 3091-3100 (2000).

### Mouse Embryos

Mouse embryos were dissected into DEPC-treated PBS, fixed overnight in 4% PFA, and dehydrated into MeOH. In situ hybridization used Id1, Grrp1, Evx1 (cloned into pGEM), and Mesp1 (Saga et al., 1996) probes (60° C. hybridization) as described, e.g., in Wilkinson et al., “Detection of messenger RNA by in Situ hybridization to tissue sections and whole mounts,” Methods in Enzymology, 225: 361-373 (1993). For histology, embryos were embedded in paraffin, H&E stained and sectioned (5 μm thickness) following standard procedures. Sections were scanned at high magnification (40×) using Leica Aperio AT2.

### CRISPR/Cas9 Id Gene Editing of Mouse Embryos

CRISPR/Cas9 gene editing to generate Id1-4 mutant embryos was performed. Eight single-guide RNAs (sgRNAs) were designed to target sites near the ATG translation initiation site and near the beginning of the HLH domain for each Id gene, using the tool at crispr.mit.edu to ensure maximum specificity. DNA templates for sgRNAs were generated by PCR amplification (Phusion DNA Polymerase; New England Biolabs, Ipswich, MA) of ssDNA ultramer oligonucleotides (Integrated DNA Technologies, Coralville, IA); sgRNAs were transcribed from these templates using HiScribe T7 High Yield RNA Synthesis Kit (New England Biolabs, Ipswich, MA) and purified using Megaclear Kit (Life Technologies, Carlsbad, CA). For mouse zygote injections, 50 Cas9 mRNA (Life Technologies, Carlsbad, CA) and 20 ng/μL of each sgRNA was combined in nuclease free water. Fertilized oocytes were collected from 3 to 4 week-old superovulated C57B16 females (prepared by injecting 5 IU each of pregnant mare serum gonadotropin and human chorionic gonadotropin (Sigma-Aldrich, St. Louis, MO)), then transferred into M2 medium (Millipore, Billerica, MA), and injected with the Cas9 mRNA/sgRNA solution into the cytoplasm. Injected embryos were then re-implanted into recipient pseudo-pregnant ICR female mice. Implanted females were sacrificed 8-9 days after re-implantation; yolk sac DNA was collected for genotyping by PCR (Bioline MyTaq Extract kit) followed by DNA deep sequencing (Ilumina Nextera kit for library preparation, Illumina HiSeq 1500 for sequencing). Sequences were analyzed, and variant alleles were recorded, using IGV genome browser (Broad Institute, Cambridge, MA). For off-target analysis, the top 8 off-target sites were identified using the tool at crispr.mit.edu; these regions were PCR amplified and Sanger sequenced.

### Statistics

Each experiment represents at least quadruplicate biological replicates per condition. Statistical analysis was performed with unpaired Student's T test, P<0.05.

### Example 2: Identification of New Agonists of Cardiogenic Mesoderm Formation

Mouse embryonic stem cells (mESCs) form mesendodermal progenitors (Gsc⁺, Foxa2⁺, T⁺) at day 3-4 of differentiation in response to Activin/Nodal signaling and subsequently differentiate into either Foxa2⁺ definitive endoderm or Kdr⁺ cardiogenic mesoderm (diagrammed in FIG. 1A). Attenuation of Acvr1b drives mesendodermal progenitors to form multipotent cardiovascular progenitors marked by Mesp1, Kdr, Cdh11, and Snai1 expression at day 5-6, rather than endoderm; a process robustly elicited by transfecting mesendodermal progenitors at day 3 with either let-7 or miR-18 mimics or siRNAs directed against their respective mRNA targets Acvr1b or Smad2 (day 3) (FIGS. 1A-1C).

In order to identify the downstream effectors of cardiogenic mesoderm formation, mRNA expression was analyzed 24 hours after Acvr1b siRNA (siAcvr1b) transfection (Day 4). Microarray data revealed 33 genes that were upregulated (FIG. 1D and Table 2) in response to Acvr1b siRNA relative to a scrambled sequence siRNA control, of which 14 were confirmed by qPCR (FIG. 1E). Consistent with a potential role as cell fate regulators, 8 of the candidate genes are known regulators of gene transcription, including transcription factors (Evx1, Gbx2, Irx3, Irx5, Sox9), inhibitors of bHLH transcription factors (Id1, Id3), and a mediator of DNA demethylation (Gadd45g). Of the 6 remaining candidates, 3 are signaling pathway modulators (Fgfbp3, Crabp2, Cxcl12), 2 are involved in RNA processing (Elavl3, Tnrc6a), and 1 encodes a protein with two centrosome-associated domains but no known function (Grrp1). Interestingly, none of the 14 candidates were previously shown to directly control cardiogenic mesoderm formation, suggesting that a novel molecular signature marking differentiating multipotent cardiovascular progenitors was identified.

Next, siRNA against each of the 14 candidates was assessed whether it would block cardiogenic mesoderm formation induced by siAcvr1b using a Kdr-eGFP reporter system (Colas et al., 2012). Of all the upregulated genes, only siRNAs against Grrp1, Evx1, and Id1 significantly decreased the number of Kdr⁺-expressing cells (FIGS. 1F-1J) and blunted the induction of cardiogenic mesoderm marker genes including Kdr, Mesp1, Snai1, and Cdh11 (FIG. 1K). Thus, Grrp1, Evx1, and Id1 are needed for normal cardiogenic mesoderm differentiation in mESCs.

TABLE 2
Gene Candidates Regulated by siAcvr1b

| | Fold | | | Fold | |
|---|---|---|---|---|---|
| | Upregulation | | | Downregulation | |
| | P-Value < 0.05 | Gene ID | | P-Value < 0.05 | Gene ID |
| 1 | 2.609411 | Id1 | 1 | 1.6015979 | Zmpste24 |
| 2 | 2.0134268 | Gadd45g | 2 | 1.6013767 | Cdc42 |
| 3 | 1.5968683 | Irx3 | 3 | 1.5671402 | Egr1 |
| 4 | 1.4899422 | Sox9 | 4 | 1.5383662 | Fgf8 |
| 5 | 1.4583107 | Evx1 | 5 | 1.525815 | Leftb |
| 6 | 1.4510411 | Cxcl12 | 6 | 1.515485 | Acvr1b |
| 7 | 1.4346725 | Rgma | 7 | 1.4910644 | Zic2 |
| 8 | 1.4037634 | Tnrc6a | 8 | 1.4896251 | Ppp4r4 |
| 9 | 1.3917822 | Gadd45g | 9 | 1.467095 | Gemin6 |
| 10 | 1.3839511 | Gbx2 | 10 | 1.4522598 | Trim67 |
| 11 | 1.3793361 | BC030476 | 11 | 1.4373533 | Notch3 |
| 12 | 1.3663218 | Fus | 12 | 1.4351145 | Srprb |
| 13 | 1.365738 | Irx5 | 13 | 1.4311734 | Gsc |
| 14 | 1.3544436 | Elavl3 | 14 | 1.4305644 | Zmpste24 |
| 15 | 1.3542395 | Crabp2 | 15 | 1.4290521 | Igfbp3 |
| 16 | 1.3431355 | 1500011K16Rik | 16 | 1.4245033 | Zmpste24 |
| 17 | 1.3422825 | Tnrc6 | 17 | 1.4184229 | Grm6 |
| 18 | 1.3389522 | Ccdc85b | 18 | 1.4032689 | Ube2q |
| 19 | 1.329755 | Grrp1 | 19 | 1.3981106 | Lbr |
| 20 | 1.3260252 | Fgfbp3 | 20 | 1.3852041 | Tmem63a |
| 21 | 1.3247453 | Bcl2l11 | 21 | 1.3845202 | Pitx2 |
| 22 | 1.3216742 | Slc1a3 | 22 | 1.382626 | Npm3 |
| 23 | 1.3198931 | Gtl2 | 23 | 1.3775356 | Ttc19 |
| 24 | 1.3195066 | Id3 | 24 | 1.3773962 | Stk4 |
| 25 | 1.3176388 | Bckdha | 25 | 1.3745593 | Sgk |
| 26 | 1.3164426 | Chka | 26 | 1.3733177 | Armcx2 |
| 27 | 1.3119096 | Chd4 | 27 | 1.365576 | Tmem63a |
| 28 | 1.3110358 | Rras | 28 | 1.3652078 | Wdr82 |
| 29 | 1.3094118 | Mrg1 | 29 | 1.3617791 | Gemin6 |
| 30 | 1.3044847 | Lsm12 | 30 | 1.3600438 | Ppp4r4 |
| 31 | 1.3036366 | Cbln1 | 31 | 1.359722 | Tcn2 |
| 32 | 1.3035693 | Zfp296 | 32 | 1.3578465 | Dnajc6 |
| 33 | 1.3024908 | Klf7 | 33 | 1.3522204 | Ncoa4 |
| | | | 34 | 1.3519416 | Prpf8 |
| | | | 35 | 1.3507187 | Hdlbp |
| | | | 36 | 1.346875 | Rnf213 |
| | | | 37 | 1.3434738 | Nodal |
| | | | 38 | 1.3346514 | Slc19a2 |
| | | | 39 | 1.3340039 | Rab1 |
| | | | 40 | 1.332933 | Klhl22 |
| | | | 41 | 1.3268061 | Foxa2 |
| | | | 42 | 1.3243924 | Zfp750 |
| | | | 43 | 1.323197 | Map2k4 |
| | | | 44 | 1.3197291 | Eppk1 |
| | | | 45 | 1.3165845 | Car2 |
| | | | 46 | 1.3164321 | Smarca5-ps |
| | | | 47 | 1.3136374 | Lefty1 |
| | | | 48 | 1.3131495 | Cnn3 |
| | | | 49 | 1.3128709 | Igfbp3 |
| | | | 50 | 1.311379 | Ints5 |
| | | | 51 | 1.30427 | Tgfbr3 |
| | | | 52 | 1.3032453 | Chst15 |
| | | | 53 | 1.3031058 | Atl2 |

### Example 3: Spatiotemporal Expression of Id1, Grrp1 and Evx1 is Consistent with Involvement in Cardiogenic Mesoderm Formation

Maximal Id1 expression occurs at day 4 of mESC differentiation, preceding the peaks of Grrp1, Evx1, and Mesp1 expression (FIGS. 1L-1O). In mouse embryos, Id1 is expressed throughout the entire epiblast of the proximal region of the late gastrula-stage (E7.5) embryo near the primitive streak, and is also strongly expressed in lateral mesoderm as it migrates toward the anterior region of the embryo where early specified cardiac precursors are located (FIGS. 1P, 1T, 1T′) (Devine et al 2014). Id1 transcripts are notably absent from the primitive streak, posterior mesoderm, and definitive endoderm. Grrp1 transcripts are expressed throughout the primitive streak of the embryo (FIGS. 1Q, 1U, 1U′). Transverse sections reveal that Grrp1 expression is mostly localized in gastrulating epiblast and rapidly declines as cells migrate away from the primitive streak. Evx1 expression is absent from the gastrulating epiblast while being mostly concentrated in the primitive streak and migrating mesoderm (FIGS. 1R, 1V, 1V′). Evx1 expression greatly decreases as cells migrate towards the anterior region of the embryo. Mesp1 expression marks early differentiating multipotent mesoderm, and is expressed by cells as they emerge from the primitive streak and start to migrate (FIGS. 1S, 1W, 1W′). Thus, spatiotemporal expression of candidate transcripts is consistent with their potential involvement in cardiogenic mesoderm specification, and also suggests that Id1 and Grrp1 in the gastrulating epiblast may function upstream of Evx1 in the primitive streak to ultimately direct Mesp1 expression in cells that exit the primitive streak (FIG. 1X).

### Example 4: Id1 is Sufficient to Direct Kdr+ Cardiogenic Mesoderm Formation in Mouse and Human ESCs

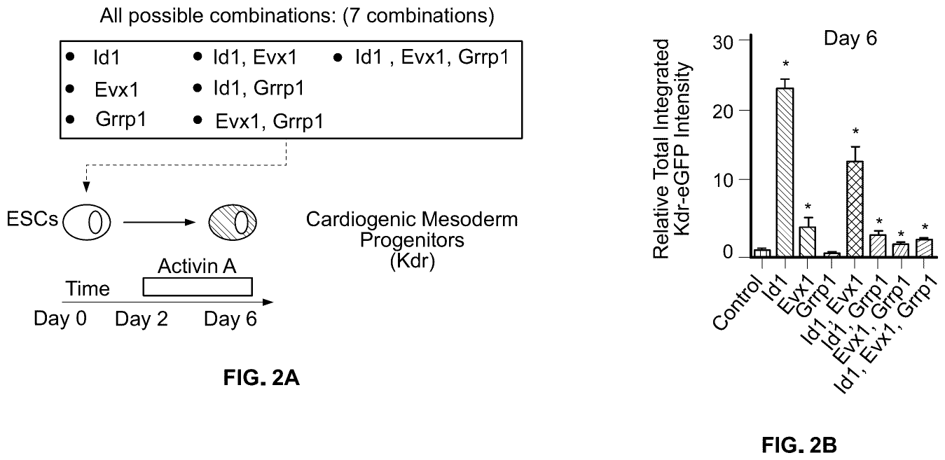

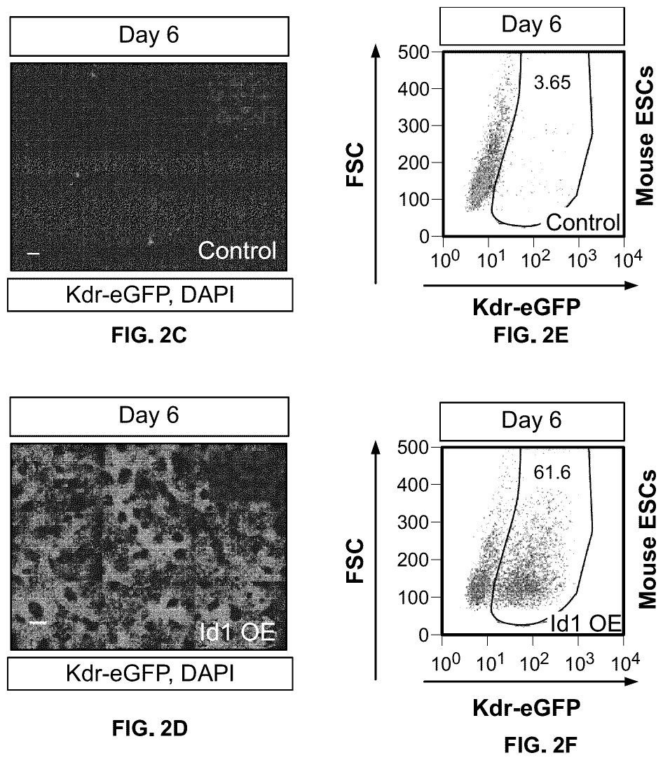

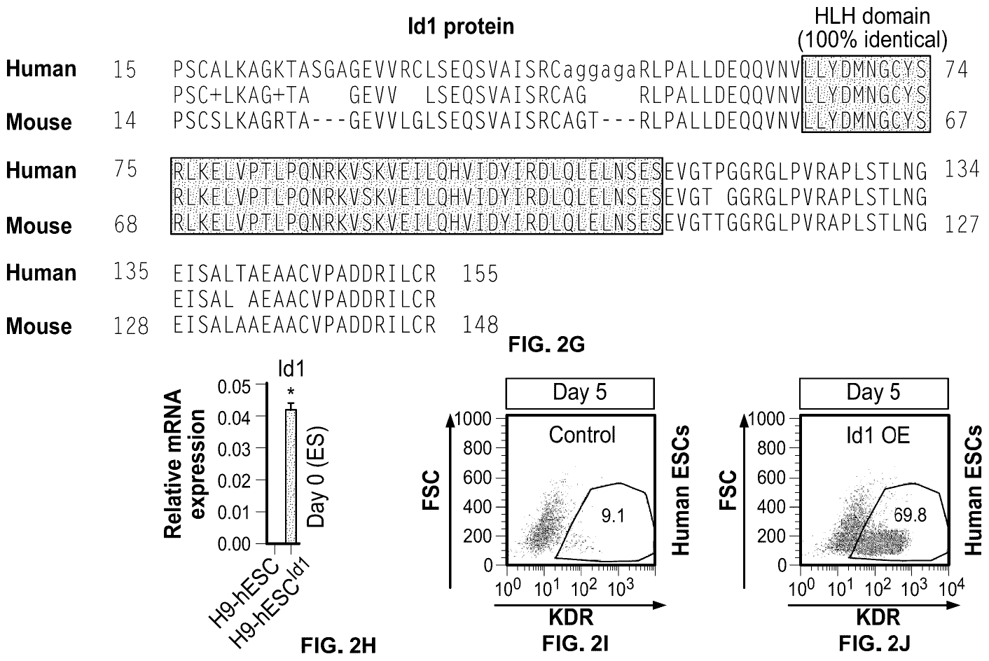

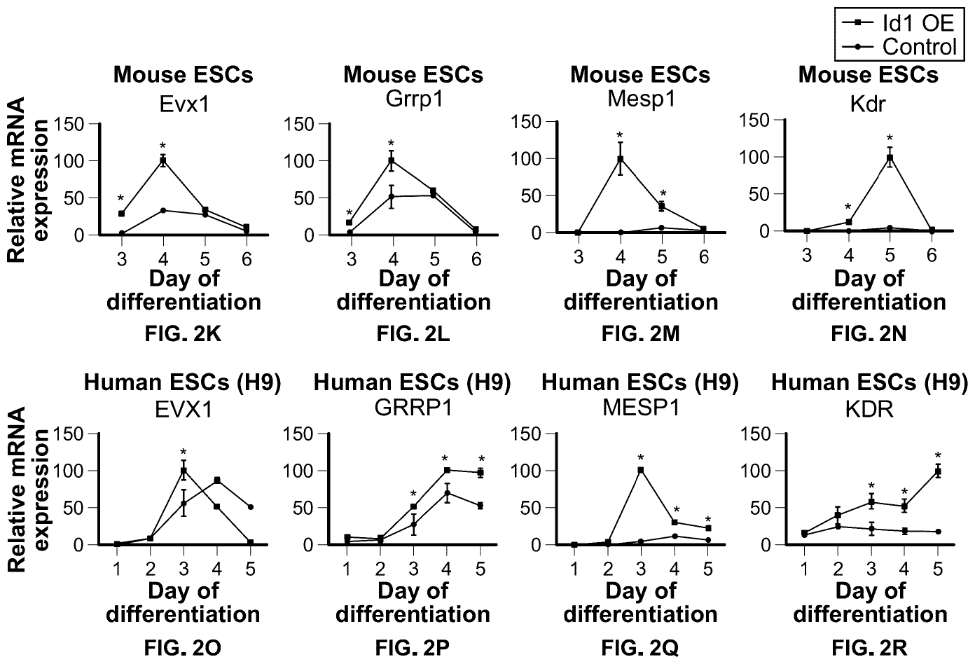

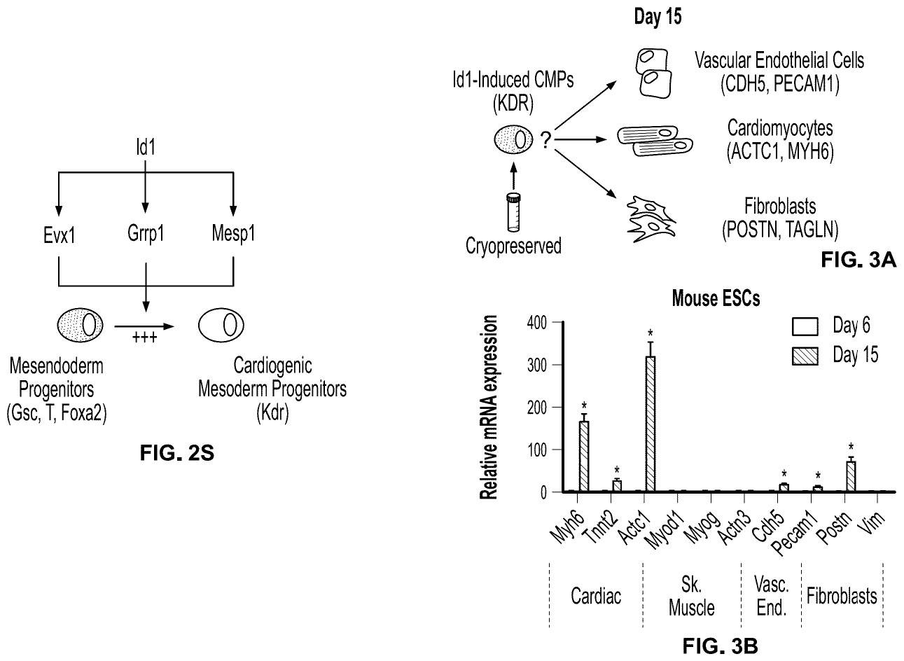

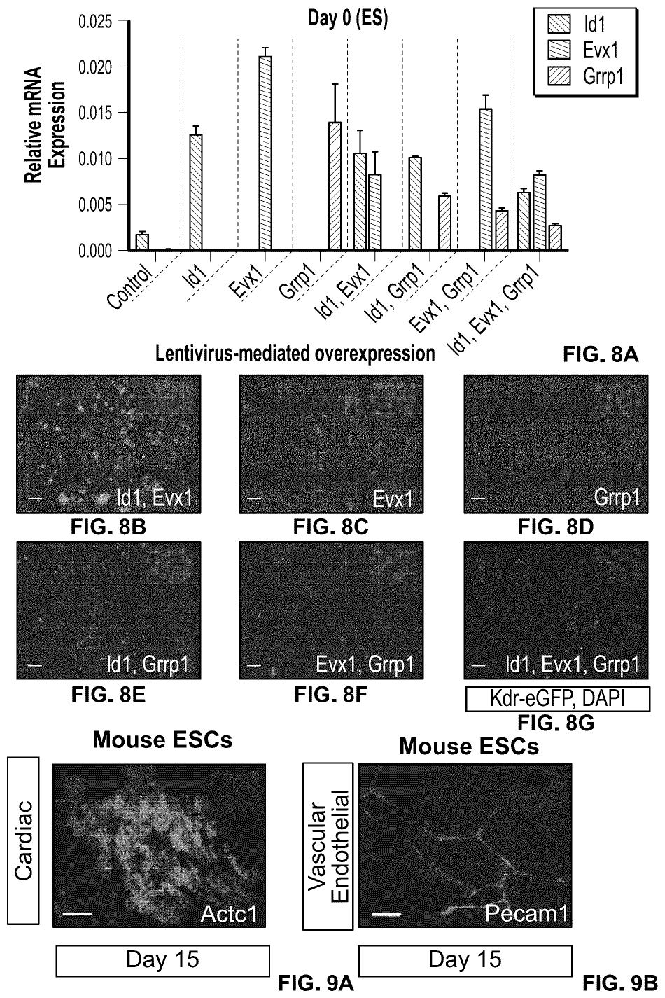

In order to evaluate whether candidate genes, alone or in combination, are sufficient to promote cardiogenic mesoderm differentiation, mESC lines overexpressing all 7 possible combinations of the 3 candidates were generated (FIG. 2A and FIG. 8A). The cell lines were treated with Activin A (but not with Acvr1b siRNA) and the resulting differentiation was assessed on day 6. Id1 was sufficient to massively direct ESCs to differentiate towards Kdr⁺ mesoderm without Acvr1b attenuation (˜22-fold over parental mESCs), while the other genes had less potent effects (FIGS. 2B-2D and FIGS. 8B-8G). Quantitatively, the conversion rate of Id1-overexpressing mESCs into Kdr-eGFP⁺ mesoderm is approximately 60% as compared to only 3.65% for control ESCs (FIGS. 2E, 2F).

Next, experiments were performed to determine whether Id1 functions similarly in human ESCs (hESCs) by generating a WiCell (H9) hESC line that stably overexpresses mouse Id1 since mouse and human HLH domains are identical (FIGS. 2G, 2H). Consistent with mESCs, Id1 greatly increased the incidence of KDR⁺-mesoderm in Activin A treated cultures at day 5 from 9.1% in parental hESCs to 69.8% in hESC^{Id1} (FIGS. 2I, 2J).

Remarkably, the formation of Id1-induced Kdr⁺/KDR⁺ mesoderm progenitors (iMPs) was consistently preceded by the upregulation of Evx1/EVX1 and Grrp1/GRRP1 (day 3/4 in mESCs (FIGS. 2K, 2L) and day 3 in hESCs (FIGS. 2O, 2P)); followed by dramatic Mesp1/MESP1 upregulation (FIGS. 2M, 2Q; ˜67 fold in mESCs at day 4 and ˜20 fold in hESCs at day 3); and subsequent Kdr/KDR upregulation at day 4 and day 5, respectively (FIGS. 2N, 2R). Altogether, these data show that Id1 initiates the activation of an evolutionarily conserved gene regulatory network (Evx1/EVX1, Grrp1/GRRP1 and Mesp1/MESP1) controlling the formation Kdr⁺/KDR⁺ mesoderm (FIG. 2S).

Next, experiments were performed to determine whether iMP progenitors are bona fide multipotent cardiovascular progenitors and thus able to differentiate into multiple cardiac lineages including functional cardiomyocytes. To address this question, iMPs were first produced in bulk until day 6 of differentiation for mouse, or day 5 for human. At this point, iMPs could be cryopreserved or used fresh. Spontaneous differentiation potential under basal media conditions without cytokines (FIG. 3A) was assessed by RT-qPCR (FIGS. 3B, 3C) and immunostaining (FIG. 3D and FIGS. 9A, 9B) at day 15 of differentiation. The results show that iMPs spontaneously differentiate into at least three distinct cellular lineages normally present in the heart, including cardiomyocytes (Myh6, Tnnt2, Actc1), vascular endothelial cells (Pecam1, Cdh5), and fibroblasts (Postn, Tagln) in both species. Although iMPs are multipotent progenitors, the vast majority of the cells (˜70%) spontaneously differentiate into ACTC1⁺ cardiomyocytes in hESCs (FIG. 3E). Next, we assessed whether resulting ACTC1⁺ cells show characteristics of functional cardiomyocytes, which include the ability to contract, display intracellular calcium oscillations and action potentials, and respond to hormonal stimuli (Birket et al., 2015; Burridge et al., 2014). High-speed optical recording (100 frames per second) (FIG. 3F) reveals that in addition to expressing cardiac-specific markers, day 15 cells contract rhythmically (Movie S3), display periodic calcium transients (FIGS. 3G, 3H and Movie S4) and action potentials, (FIG. 3I and Movie S5) and show increased beat rate in response to B-adrenergic agonist, isoproterenol (FIGS. 3J, 3K and Movie S6). In summary, these observations demonstrate that iMPs represent a novel population of bona fide multipotent cardiovascular progenitors with remarkable ability to spontaneously differentiate into functional cardiomyocytes.

### Example 5: Id1 Promotes Cardiogenic Mesoderm Differentiation by Inhibiting Tcf3 and Foxa2

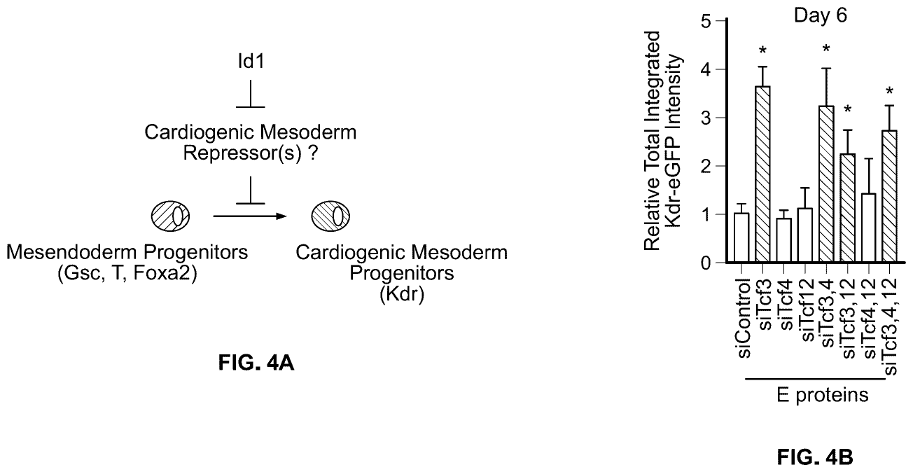

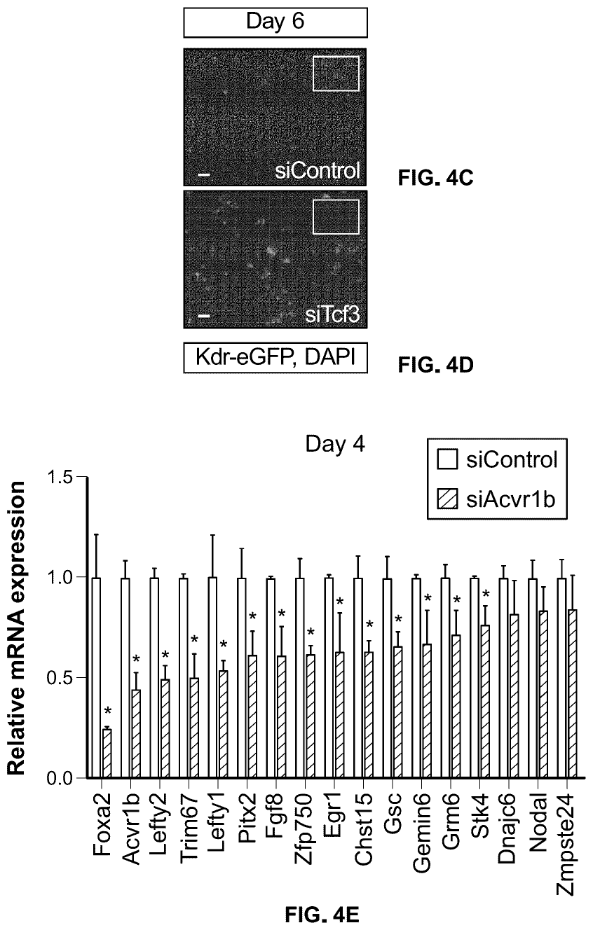

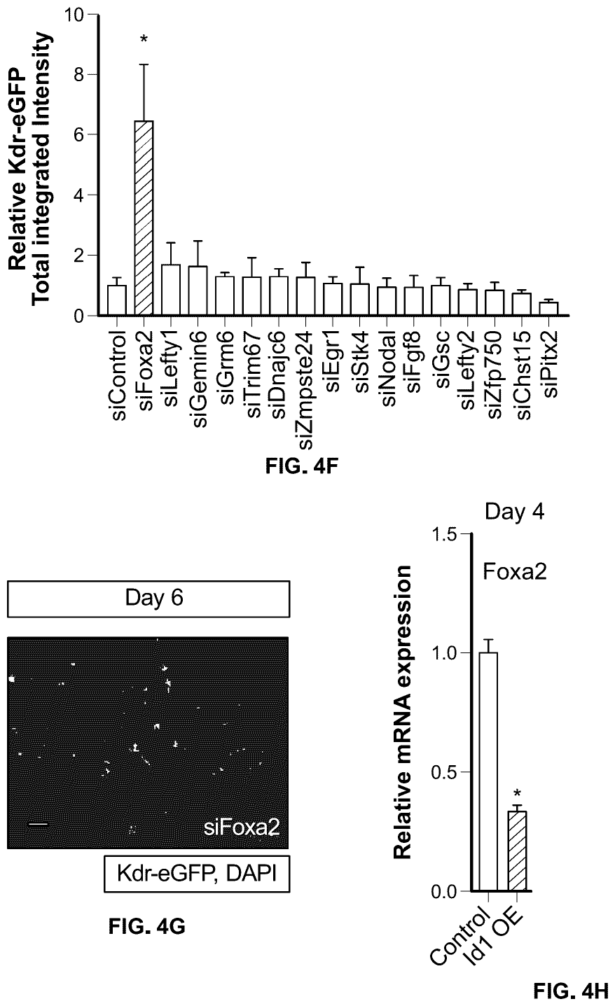

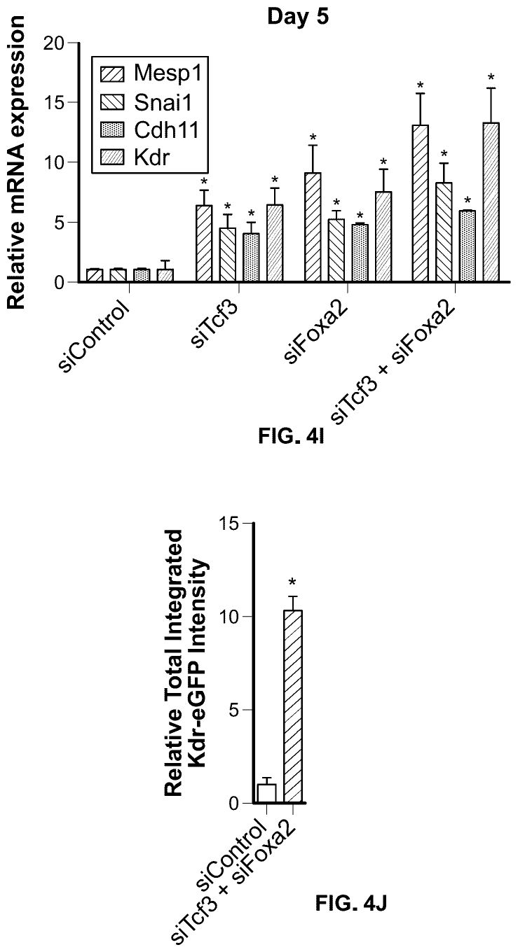

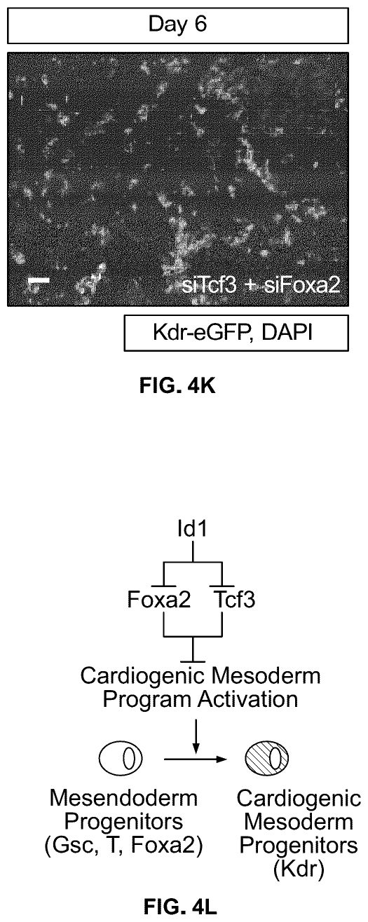

Id proteins do not bind DNA directly, but regulate transcription by antagonizing the function of bHLH transcription factors through their HLH domains (Kee, 2009). Their canonical partners are the ubiquitously expressed class I bHLH transcription factors (E proteins) Tcf3, Tcf4 and Tcf12 (Kee, 2009; Yang et al., 2014). Therefore, to determine if Id1 might initiate cardiogenic mesoderm formation by inhibiting E proteins (FIG. 4A), experiments were performed to test if siRNAs directed against the three E proteins, either alone or in combination (7 combinations) would inhibit Kdr-eGFP fluorescence at day 6 of differentiation as above. All combinations of siRNAs that contained siTcf3 promoted cardiogenic mesoderm differentiation (˜4-fold over siControl) (FIGS. 4B-4D). Although these studies implicate Tcf3 as a relevant target of Id1, siTcf3 was significantly less potent at inducing Kdr-eGFP⁺ cells than either Id1 overexpression or siAcvr1b transfection, suggesting that additional targets are involved. Therefore, all 104 members of the class II family of bHLH transcription factors were screened (e.g. MyoD, NeuroD, myogenin, etc.) by an analogous approach, but none had any effect on cardiogenic mesoderm formation.

Next, experiments were performed to test whether Id1 might mediate part of its pro-cardiogenic mesoderm activity by downregulating antagonists of cardiogenic mesoderm formation. Such genes should be among those downregulated in response to the pro-cardiogenic mesoderm actions of siAcvr1b at day 4 of differentiation. Out of the 53 genes identified in the microarray (Table 2), 17 were confirmed by RT-qPCR to be robustly downregulated by siAcvr1b (FIG. 4E). Next, experiments were performed to test whether siRNA-mediated knockdown of any of these 17 genes would be sufficient to promote Kdr-eGFP⁺ cardiogenic mesoderm formation. Strikingly, siRNA to only one gene, encoding the forkhead transcription factor, Foxa2, was sufficient to induce Kdr-eGFP⁺ mesoderm (FIGS. 4F, 4G). Although Id1 is not known to physically interact with forkhead transcription factors, overexpression of Id1 strongly decreased the abundance of Foxa2 transcripts in the cells relative to controls (FIG. 4H), suggesting that Id1 indirectly inhibits Foxa2 gene expression.

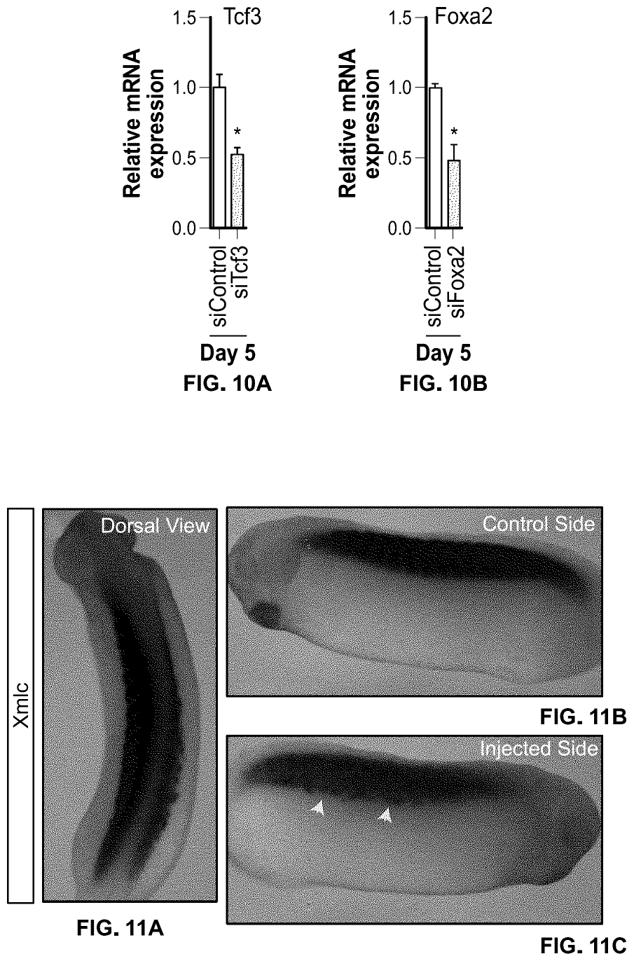

Moreover and consistent with the hypothesis, Tcf3 and Foxa2 knockdowns (FIGS. 10A, 10B) each de-repressed cardiogenic mesoderm gene expression (Mesp1, Snai1, Cdh11, and Kdr) (FIG. 4I). In addition, combined knockdown of Tcf3 and Foxa2 further enhanced cardiogenic mesoderm differentiation efficiency, suggesting that both genes act in a non-redundant manner during this process (FIGS. 4I-4K). Thus, Id1 activates the cardiogenic program by inhibiting Tcf3 protein activity while suppressing Foxa2 transcription (FIG. 4L).

### Example 6: Id Proteins Promote Cardiogenic Mesoderm Formation in Vivo

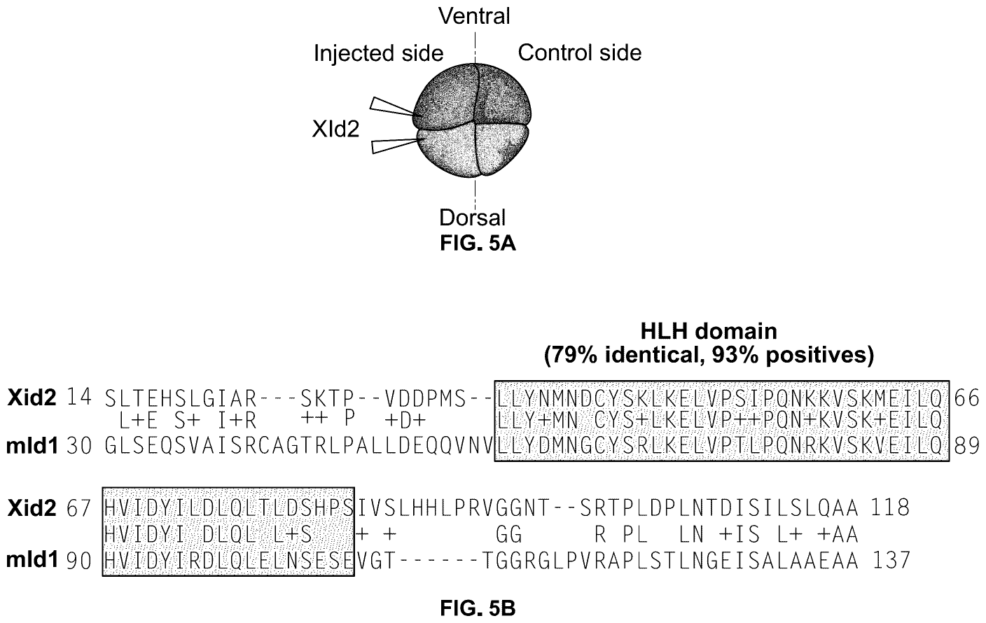

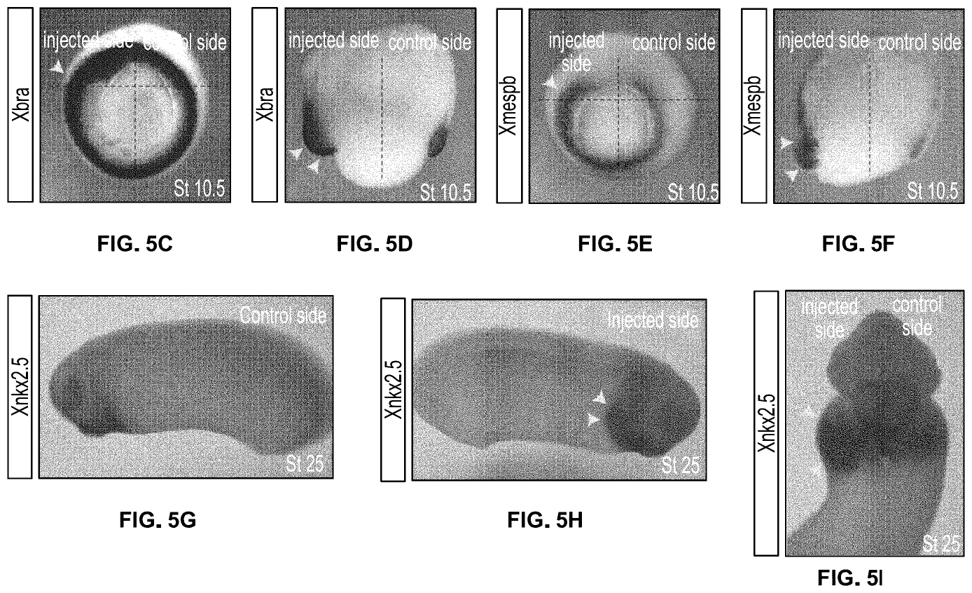

Next, Xenopus embryos were used to test if Id genes can promote cardiogenic mesoderm formation in vivo. Equatorial and hemilateral injection of Xid2 mRNA (FIG. 5A), which is the closest orthologue to mouse Id1 (FIG. 5B), causes a dramatic expansion of Xbra (FIGS. 5C, 5E; 74%, n=105) and Xmespb (FIGS. 5D, 5F; 78%, n=132) expression domains (marking mesoderm) in gastrula-stage embryos (Nieuwekoop and Faber stage 10.5). To determine if the expanded Xbra⁺, Xmespb⁺ domains correlate with a subsequent increase in cardiogenesis, tailbud stage (stage 25) embryos were examined for Xnkx2.5 expression that marks the cardiac primordium (Raffin et al., 2000). Strikingly, Xid2 overexpression caused an expansion of Xnkx2.5 expression domain (FIGS. 5G-5I; 68%, n=88) while in contrast, it diminished expression of the skeletal muscle marker, Xmlc (FIG. 11A-11C; 66%, n=30)). Taken together, Xid2, like mammalian Id1, promotes the formation of mesoderm progenitors that are primed to differentiate towards cardiogenic lineages.

### Example 7: Id Genes are Essential for Early Mammalian Heart Formation in Vivo

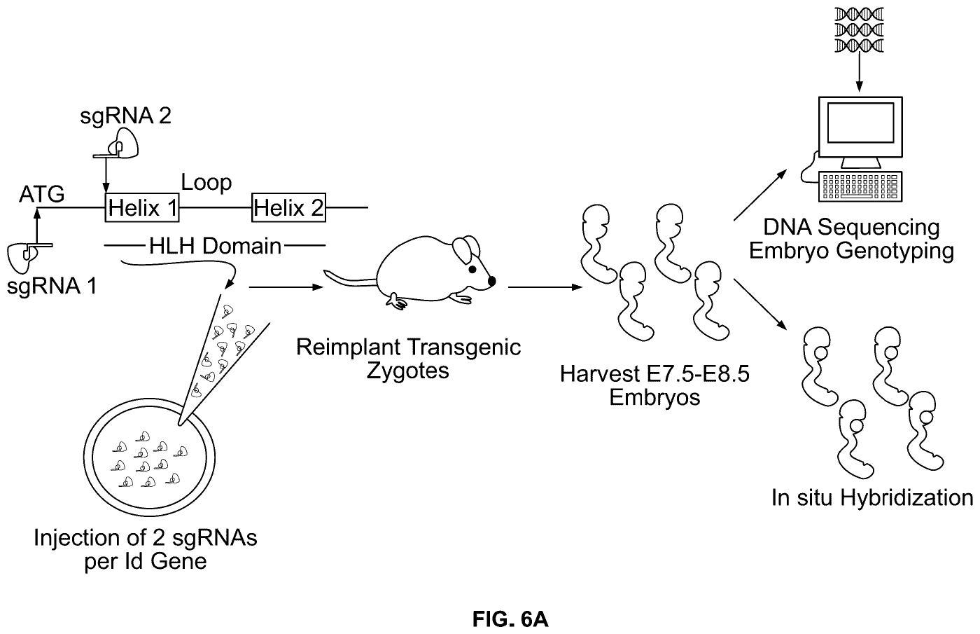

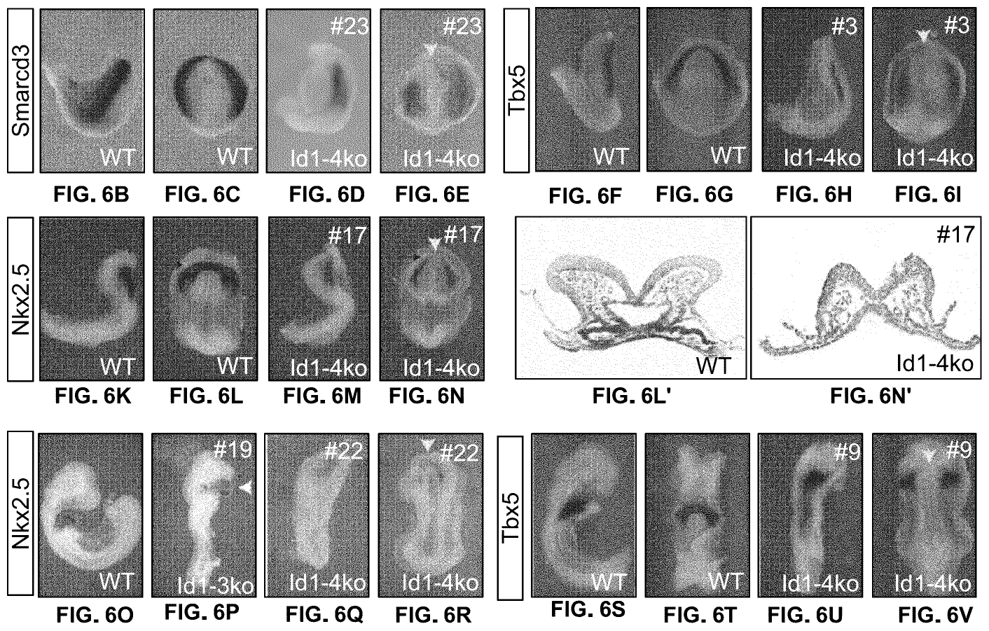

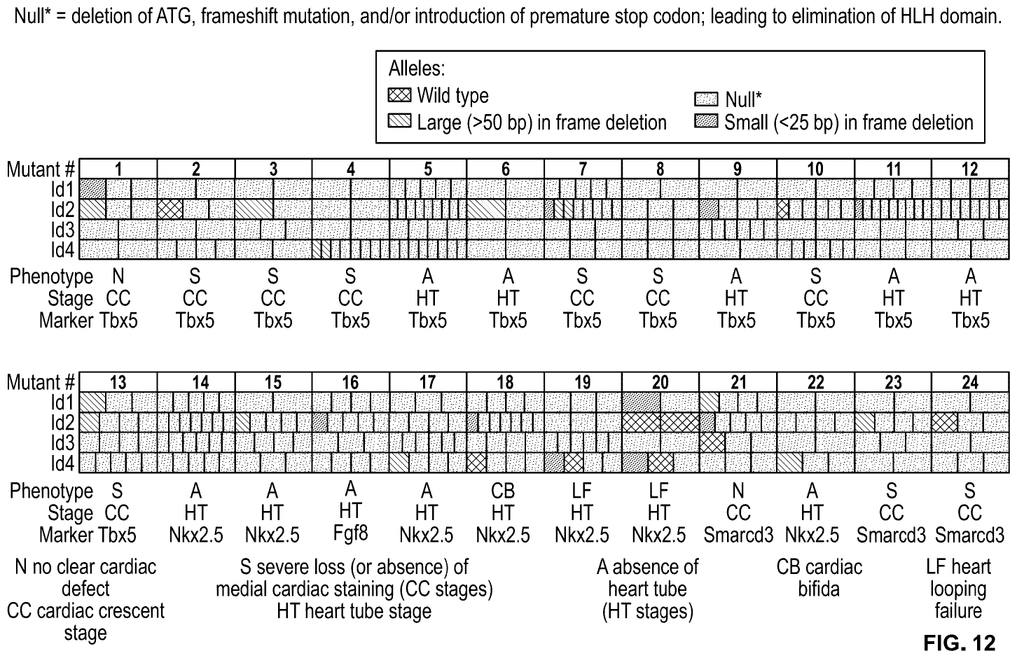

The gain of function experiments show that Id proteins are sufficient to direct the formation of multipotent cardiac progenitors both in vitro and in vivo, so the next question is whether Id proteins are normally required for this process. A previous study (Fraidenraich et al., 2004) found that deleting 3 out of the 4 Id genes (Id1, 2, 3 triple gene knockout) caused complex cardiac defects, but did not ablate the heart in these embryos, thereby indicating that earlier cardiac specification could still occur. Given the functional similarity of Id family members (Fraidenraich et al., 2004; Kee and Bronner-Fraser, 2005; Lyden et al., 1999; Niola et al., 2012; Niola et al., 2013), it was hypothesized that either redundant or compensatory activity of Id4 might allow heart formation to occur in triple knockout embryos. To test this hypothesis, all four Id gene members were genetically ablated using a CRISPR/Cas9 gene editing strategy in mouse embryos. To increase the probability of null allele generation, each Id gene was targeted by two sgRNAs, directed against the ATG and the beginning of the HLH domain, respectively. 24 embryos (ranging from E7.75-E8.75), collected from three independent zygote injection sessions, were subjected to genotyping by DNA sequencing and cardiac phenotype assessment by in situ hybridization (FIG. 6A). DNA sequencing results show that despite widespread mosaicism, 320 (90.7%) of the 353 alleles detected were null (elimination of the HLH-domain reading frame), 24 (6.8%) were in-frame mutations, and only 9 (2.5%) were wild-type. Only 7/24 embryos harbored one or more wild-type alleles while 17/24 embryos harbored no wild-type alleles (FIG. 12). Importantly, no off-target mutagenesis was detected in the top 8 predicted off-target sites.

The phenotypic assessment at E7.75 showed that two markers of early cardiac precursors, Smarcd3 and Tbx5, were absent from the most anterior and medial region of the cardiac crescent that gives rise to the heart tube (FIGS. 6B-6I; n=9/11; embryos #21,23,24 (Smarcd3) and #1-3,4,7,8,10,13 (Tbx5); see FIG. 12 for genotype information). In contrast, expression of these markers was maintained in two lateral domains of mesoderm posterior to the heart tube-forming region suggesting that these posterior cardiac progenitors could differentiate and migrate appropriately. At E8.25, when the heart tube has normally formed, the cardiac marker Nkx2.5 revealed an absence of heart tube formation in Id1-4 mutants (FIGS. 6K-6N). Histological sectioning confirmed the absence of anatomical heart tube formation (FIGS. 6L′, 6N′). At E8.75, when the heart begins to loop, analysis of Nkx2.5 (FIGS. 6O, 6Q, 6R) and the first heart field marker Tbx5, (FIGS. 6S-6V) confirmed the absence of hearts in quadruple knockout embryos (FIGS. 6M, 6N, 6Q, 6R, 6U, 6V; n=10/13; embryos #14,15,17,22 (Nkx2.5) and #5,6,9,11,12,13 (Tbx5)). Finally, and consistent with the initial hypothesis of functional redundancy between Id family members, embryos harboring at least one Id4 wild-type allele can still form a heart tube that loops, albeit abnormally, as compared to controls (FIGS. 6O, 6P; n=3/13; embryo #18,19,20). Collectively, these results demonstrate that the Id family of genes is required for the specification of heart tube-forming multipotent cardiovascular progenitors and its subsequent assembly.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

Benezra, R., Davis, R. L., Lockshon, D., Turner, D. L., and Weintraub, H. (1990). The protein Id: a negative regulator of helix-loop-helix DNA binding proteins. Cell 61, 49-59.
Beppu, H., Kawabata, M., Hamamoto, T., Chytil, A., Minowa, O., Noda, T., and Miyazono, K. (2000). BMP type II receptor is required for gastrulation and early development of mouse embryos. Developmental biology 221, 249-258.
Birket, M. J., Ribeiro, M. C., Verkerk, A. O., Ward, D., Leitoguinho, A. R., den Hartogh, S. C., Orlova, V. V., Devalla, H. D., Schwach, V., Bellin, M., et al. (2015). Expansion and patterning of cardiovascular progenitors derived from human pluripotent stem cells. Nature biotechnology 33, 970-979.
Bondue, A., Lapouge, G., Paulissen, C., Semeraro, C., Iacovino, M., Kyba, M., and Blanpain, C. (2008). Mesp1 acts as a master regulator of multipotent cardiovascular progenitor specification. Cell stem cell 3, 69-84.
Bruneau, B. G. (2013). Signaling and transcriptional networks in heart development and regeneration. Cold Spring Harb Perspect Biol 5, a008292.
Buckingham, M., Meilhac, S., and Zaffran, S. (2005). Building the mammalian heart from two sources of myocardial cells. Nature reviews Genetics 6, 826-835.
Burridge, P. W., Matsa, E., Shukla, P., Lin, Z. C., Churko, J. M., Ebert, A. D., Lan, F., Diecke, S., Huber, B., Mordwinkin, N. M., et al. (2014). Chemically defined generation of human cardiomyocytes. Nature methods 11, 855-860.
Cai, C. L., Liang, X., Shi, Y., Chu, P. H., Pfaff, S. L., Chen, J., and Evans, S. (2003). Isl1 identifies a cardiac progenitor population that proliferates prior to differentiation and contributes a majority of cells to the heart. Developmental cell 5, 877-889.
Chan, S. S., Shi, X., Toyama, A., Arpke, R. W., Dandapat, A., Iacovino, M., Kang, J., Le, G., Hagen, H. R., Garry, D. J., et al. (2013). Mesp1 patterns mesoderm into cardiac, hematopoietic, or skeletal myogenic progenitors in a context-dependent manner. Cell stem cell 12, 587-601.
Chiapparo, G., Lin, X., Lescroart, F., Chabab, S., Paulissen, C., Pitisci, L., Bondue, A., and Blanpain, C. (2016). Mesp1 controls the speed, polarity, and directionality of cardiovascular progenitor migration. J Cell Biol 213, 463-477.
Colas, A., Cartry, J., Buisson, I., Umbhauer, M., Smith, J. C., and Riou, J. F. (2008). Mix. 1/2-dependent control of FGF availability during gastrulation is essential for pronephros development in Xenopus. Developmental biology 320, 351-365.
Colas, A. R., McKeithan, W. L., Cunningham, T. J., Bushway, P. J., Garmire, L. X., Duester, G., Subramaniam, S., and Mercola, M. (2012). Whole-genome microRNA screening identifies let-7 and mir-18 as regulators of germ layer formation during early embryogenesis. Genes & development 26, 2567-2579.
Collop, A. H., Broomfield, J. A., Chandraratna, R. A., Yong, Z., Deimling, S. J., Kolker, S. J., Weeks, D. L., and Drysdale, T. A. (2006). Retinoic acid signaling is essential for formation of the heart tube in Xenopus. Developmental biology 291, 96-109.
Costello, I., Pimeisl, I. M., Drager, S., Bikoff, E. K., Robertson, E. J., and Arnold, S. J. (2011). The T-box transcription factor Eomesodermin acts upstream of Mesp1 to specify cardiac mesoderm during mouse gastrulation. Nature cell biology 13, 1084-1091.
David, R., Brenner, C., Stieber, J., Schwarz, F., Brunner, S., Vollmer, M., Mentele, E., Muller-Hocker, J., Kitajima, S., Lickert, H., et al. (2008). MesP1 drives vertebrate cardiovascular differentiation through Dkk-1-mediated blockade of Wnt-signalling. Nature cell biology 10, 338-345.
Djiane, A., Riou, J., Umbhauer, M., Boucaut, J., and Shi, D. (2000). Role of frizzled 7 in the regulation of convergent extension movements during gastrulation in Xenopus laevis. Development 127, 3091-3100.
Ema, M., Takahashi, S., and Rossant, J. (2006). Deletion of the selection cassette, but not cis-acting elements, in targeted Flk1-lacZ allele reveals Flk1 expression in multipotent mesodermal progenitors. Blood 107, 111-117.
Foley, A. C., Korol, O., Timmer, A. M., and Mercola, M. (2007). Multiple functions of Cerberus cooperate to induce heart downstream of Nodal. Developmental biology 303, 57-65.
Fraidenraich, D., Stillwell, E., Romero, E., Wilkes, D., Manova, K., Basson, C. T., and Benezra, R. (2004). Rescue of cardiac defects in id knockout embryos by injection of embryonic stem cells. Science 306, 247-252.
Gadue, P., Huber, T. L., Paddison, P. J., and Keller, G. M. (2006). Wnt and TGF-beta signaling are required for the induction of an in vitro model of primitive streak formation using embryonic stem cells. Proc Natl Acad Sci USA 103, 16806-16811.
Galvin, K. E., Travis, E. D., Yee, D., Magnuson, T., and Vivian, J. L. (2010). Nodal signaling regulates the bone morphogenic protein pluripotency pathway in mouse embryonic stem cells. The Journal of biological chemistry 285, 19747-19756.
Hollnagel, A., Oehlmann, V., Heymer, J., Ruther, U., and Nordheim, A. (1999). Id genes are direct targets of bone morphogenetic protein induction in embryonic stem cells. The Journal of biological chemistry 274, 19838-19845.
Huang, P., He, Z., Ji, S., Sun, H., Xiang, D., Liu, C., Hu, Y., Wang, X., and Hui, L. (2011). Induction of functional hepatocyte-like cells from mouse fibroblasts by defined factors. Nature 475, 386-389.
Katagiri, T., Imada, M., Yanai, T., Suda, T., Takahashi, N., and Kamijo, R. (2002). Identification of a BMP-responsive element in Id1, the gene for inhibition of myogenesis. Genes to cells: devoted to molecular & cellular mechanisms 7, 949-960.
Kaltman, S. J., Huber, T. L., and Keller, G. M. (2006). Multipotent flk-1+ cardiovascular progenitor cells give rise to the cardiomyocyte, endothelial, and vascular smooth muscle lineages. Developmental cell 11, 723-732.
Kee, B. L. (2009). E and ID proteins branch out. Nature reviews Immunology 9, 175-184.
Kee, Y., and Bronner-Fraser, M. (2005). To proliferate or to die: role of Id3 in cell cycle progression and survival of neural crest progenitors. Genes & development 19, 744-755.
Kelly, R. G., Brown, N. A., and Buckingham, M. E. (2001). The arterial pole of the mouse heart forms from Fgf10-expressing cells in pharyngeal mesoderm. Developmental cell 1, 435-440.
Kelly, R. G., Buckingham, M. E., and Moorman, A. F. (2014). Heart fields and cardiac morphogenesis. Cold Spring Harb Perspect Med 4.
Korchynskyi, O., and ten Dijke, P. (2002). Identification and functional characterization of distinct critically important bone morphogenetic protein-specific response elements in the Id1 promoter. The Journal of biological chemistry 277, 4883-4891.
Laflamme, M. A., Chen, K. Y., Naumova, A. V., Muskheli, V., Fugate, J. A., Dupras, S. K., Reinecke, H., Xu, C., Hassanipour, M., Police, S., et al. (2007). Cardiomyocytes derived from human embryonic stem cells in pro-survival factors enhance function of infarcted rat hearts. Nature biotechnology 25, 1015-1024.
Lescroart, F., Chabab, S., Lin, X., Rulands, S., Paulissen, C., Rodolosse, A., Auer, H., Achouri, Y., Dubois, C., Bondue, A., et al. (2014). Early lineage restriction in temporally distinct populations of Mesp1 progenitors during mammalian heart development. Nature cell biology 16, 829-840.
Lian, X., Zhang, J., Azarin, S. M., Zhu, K., Hazeltine, L. B., Bao, X., Hsiao, C., Kamp, T. J., and Palecek, S. P. (2013). Directed cardiomyocyte differentiation from human pluripotent stem cells by modulating Wnt/beta-catenin signaling under fully defined conditions. Nature protocols 8, 162-175.
Lopez-Rovira, T., Chalaux, E., Massague, J., Rosa, J. L., and Ventura, F. (2002). Direct binding of Smad1 and Smad4 to two distinct motifs mediates bone morphogenetic protein-specific transcriptional activation of Id1 gene. The Journal of biological chemistry 277, 3176-3185.
Lyden, D., Young, A. Z., Zagzag, D., Yan, W., Gerald, W., O'Reilly, R., Bader, B. L., Hynes, R. O., Zhuang, Y., Manova, K., et al. (1999). Id1 and Id3 are required for neurogenesis, angiogenesis and vascularization of tumour xenografts. Nature 401, 670-677.
Marvin, M. J., Di Rocco, G., Gardiner, A., Bush, S. M., and Lassar, A. B. (2001). Inhibition of Wnt activity induces heart formation from posterior mesoderm. Genes & development 15, 316-327.
McKeithan, W. L., Colas, A. R., Bushway, P. J., Ray, S., and Mercola, M. (2012). Serum-free generation of multipotent mesoderm (Kdr+) progenitor cells in mouse embryonic stem cells for functional genomics screening. Current protocols in stem cell biology Chapter 1, Unit 1F 13.
Meilhac, S. M., Esner, M., Kelly, R. G., Nicolas, J. F., and Buckingham, M. E. (2004). The clonal origin of myocardial cells in different regions of the embryonic mouse heart. Developmental cell 6, 685-698.
Meilhac, S. M., Lescroart, F., Blanpain, C., and Buckingham, M. E. (2015). Cardiac cell lineages that form the heart. Cold Spring Harb Perspect Med 5, a026344.
Menasche, P., Vanneaux, V., Fabreguettes, J. R., Bel, A., Tosca, L., Garcia, S., Bellamy, V., Farouz, Y., Pouly, J., Damour, O., et al. (2015). Towards a clinical use of human embryonic stem cell-derived cardiac progenitors: a translational experience. Eur Heart J 36, 743-750.
Mercola, M., Colas, A., and Willems, E. (2013). Induced pluripotent stem cells in cardiovascular drug discovery. Circulation research 112, 534-548.
Miller, E. W., Lin, J. Y., Frady, E. P., Steinbach, P. A., Kristan, W. B., Jr., and Tsien, R. Y. (2012). Optically monitoring voltage in neurons by photo-induced electron transfer through molecular wires. Proceedings of the National Academy of Sciences of the United States of America 109, 2114-2119.
Moretti, A., Laugwitz, K. L., Dorn, T., Sinnecker, D., and Mummery, C. (2013). Pluripotent stem cell models of human heart disease. Cold Spring Harb Perspect Med 3.
Nieuwkoop, P. D. (1967). The “organization centre”. 3. Segregation and pattern formation in morphogenetic fields. Acta Biotheor 17, 178-194.
Niola, F., Zhao, X., Singh, D., Castano, A., Sullivan, R., Lauria, M., Nam, H. S., Zhuang, Y., Benezra, R., Di Bernardo, D., et al. (2012). Id proteins synchronize sternness and anchorage to the niche of neural stem cells. Nature cell biology 14, 477-487.
Niola, F., Zhao, X., Singh, D., Sullivan, R., Castano, A., Verrico, A., Zoppoli, P., Friedmann-Morvinski, D., Sulman, E., Barrett, L., et al. (2013). Mesenchymal high-grade glioma is maintained by the ID-RAP1 axis. The Journal of clinical investigation 123, 405-417.
Olson, E. N. (2006). Gene regulatory networks in the evolution and development of the heart. Science 313, 1922-1927.
Paige, S. L., Thomas, S., Stoick-Cooper, C. L., Wang, H., Mayes, L., Sandstrom, R., Pabon, L., Reinecke, H., Pratt, G., Keller, G., et al. (2012). A temporal chromatin signature in human embryonic stem cells identifies regulators of cardiac development. Cell 151, 221-232.
Pandur, P., Lasche, M., Eisenberg, L. M., and Kuhl, M. (2002). Wnt-11 activation of a non-canonical Wnt signalling pathway is required for cardiogenesis. Nature 418, 636-641.
Peng, H. B. (1991). Xenopus laevis: Practical uses in cell and molecular biology. Solutions and protocols. Methods Cell Biol 36, 657-662.
Raffin, M., Leong, L. M., Rones, M. S., Sparrow, D., Mohun, T., and Mercola, M. (2000). Subdivision of the cardiac Nkx2.5 expression domain into myogenic and nonmyogenic compartments. Developmental biology 218, 326-340.
Roschger, C., and Cabrele, C. (2017). The Id-protein family in developmental and cancer-associated pathways. Cell Commun Signal 15, 7.
Saga, Y., Hata, N., Kobayashi, S., Magnuson, T., Seldin, M. F., and Taketo, M. M. (1996). MesP1: a novel basic helix-loop-helix protein expressed in the nascent mesodermal cells during mouse gastrulation. Development 122, 2769-2778.
Saga, Y., Kitajima, S., and Miyagawa-Tomita, S. (2000). Mesp1 expression is the earliest sign of cardiovascular development. Trends in cardiovascular medicine 10, 345-352.
Schneider, V. A., and Mercola, M. (2001). Wnt antagonism initiates cardiogenesis in Xenopus laevis. Genes & development 15, 304-315.
Schultheiss, T. M., Burch, J. B., and Lassar, A. B. (1997). A role for bone morphogenetic proteins in the induction of cardiac myogenesis. Genes & development 11, 451-462.
Sekiya, S., and Suzuki, A. (2011). Direct conversion of mouse fibroblasts to hepatocyte-like cells by defined factors. Nature 475, 390-393.
Stainier, D. Y. (2002). A glimpse into the molecular entrails of endoderm formation. Genes & development 16, 893-907.
Viotti, M., Nowotschin, S., and Hadjantonakis, A. K. (2014). SOX17 links gut endoderm morphogenesis and germ layer segregation. Nature cell biology 16, 1146-1156.
Wilkinson, D. G., and Nieto, M. A. (1993). Detection of messenger RNA by in situ hybridization to tissue sections and whole mounts. Methods in enzymology 225, 361-373.
Yang, J., Li, X., and Morrell, N. W. (2014). Id proteins in the vasculature: from molecular biology to cardiopulmonary medicine. Cardiovascular research 104, 388-398.
Yang, L., Soonpaa, M. H., Adler, E. D., Roepke, T. K., Kaltman, S. J., Kennedy, M., Henckaerts, E., Bonham, K., Abbott, G. W., Linden, R. M., et al. (2008). Human cardiovascular progenitor cells develop from a KDR+embryonic-stem-cell-derived population. Nature 453, 524-528.
Yoshida, T., Vivatbutsiri, P., Morriss-Kay, G., Saga, Y., and Iseki, S. (2008). Cell lineage in mammalian craniofacial mesenchyme. Mech Dev 125, 797-808.
Zhao, R., Watt, A. J., Battle, M. A., Li, J., Bondow, B. J., and Duncan, S. A. (2008). Loss of both GATA4 and GATA6 blocks cardiac myocyte differentiation and results in acardia in mice. Developmental biology 317, 614-619.

What is claimed is:

1. A multipotent cardiovascular progenitor cell, made by a method comprising:
(i) overexpressing one or more proteins selected from the group consisting of Id1 (Inhibitor of DNA binding 1, HLH protein), Id2 (Inhibitor of DNA Binding 2, HLH Protein), Id3 (Inhibitor of DNA Binding 3, HLH Protein), Id4 (Inhibitor of DNA Binding 4, HLH Protein), Evx1 (Even-Skipped Homeobox 1), and Grrp1 (glycine/arginine rich protein 1) in a stem cell, thereby generating the multipotent cardiovascular progenitor cell; or
(ii) inhibiting the expression or activity of one or both of Foxa2 (Forkhead Box A2) and Tcf3 (Transcription Factor 3) in a stem cell, thereby generating the multipotent cardiovascular progenitor cell.

2. The multipotent cardiovascular progenitor cell of claim 1, the method further comprising transfecting the stem cell with a nucleic acid comprising a sequence encoding one or more proteins selected from the group consisting of Id1, Id2, Id3, Id4, Evx1, and Grrp1.

3. The multipotent cardiovascular progenitor cell of claim 1, the method further comprising overexpressing Mesp1 (Mesoderm posterior protein 1).

4. The multipotent cardiovascular progenitor cell of claim 1, wherein the stem cell comprises one or more proteins selected from the group consisting of Id1, Id2, Id3, Id4, Evx1, and Grrp1.

5. The multipotent cardiovascular progenitor cell of claim 1, wherein the stem cell comprises Id1, Id2, Id3, or Id4.

6. The multipotent cardiovascular progenitor cell of claim 5, wherein the stem cell comprises Id1.

7. The multipotent cardiovascular progenitor cell of claim 1, wherein the stem cell comprises Mesp1.

8. The multipotent cardiovascular progenitor cell of claim 1, wherein the stem cell is an induced pluripotent stem cell.

9. The multipotent cardiovascular progenitor cell of claim 8, wherein the stem cell is a human induced pluripotent stem cell.

10. The multipotent cardiovascular progenitor cell of claim 8, wherein the stem cell lacks one or both of Foxa2 and Tcf3 or one or both of Foxa2 and Tcf3 are at an expression level below one or more proteins selected from the group consisting of Id1, Id2, Id3, Id4, Evx1, and Grrp1.

11. The multipotent cardiovascular progenitor cell of claim 1, comprising at least one of Mesp1, Kdr, Cdh11, and Snai1.

12. The multipotent cardiovascular progenitor cell of claim 1, comprising at least one of Evx1, Grrp1 and Mesp1.
